(19) United States
(12) Patent Application Publication
(10) Pub. No.: US 2005/0118615 A1
(43) Pub. Date: Jun. 2, 2005

(54) Chordin-like homologs

(75) Inventors: Anat Oren, Tel Aviv (IL); Galit Rotman, Herzliya (IL); Amir Toporoik, Mishmar Hashiv'a (IL); Sharon Biton, Herzliya (IL); Kinneret Savitsky, Tel Aviv (IL); Jeanne Bernstein, Kfar Yona (IL)

Correspondence Address: BIRCH STEWART KOLASCH & BIRCH, PO BOX 747, FALLS CHURCH, VA, 22040-0747, US

(73) Assignee: COMPUGEN LTD., Tel Aviv (IL)

(21) Appl. No.: 10/913,447

(22) Filed: Aug. 9, 2004

Related U.S. Application Data
(63) Continuation-in-part of application No. 09/890,456, filed on Nov. 9, 2001, now abandoned.
(60) Provisional application No. 60/492,971, filed on Aug. 7, 2003.

(30) Foreign Application Priority Data
Nov. 10, 1999 (IL) 132846
Dec. 28, 1999 (IL) 133767
Nov. 10, 2000 (WO) PCT/IL00/00736

(51) Int. Cl. C12Q 1/68; G01N 33/53; A61K 38/18; A61K 48/00; C07K 14/475

(52) U.S. Cl. 435/6; 536/23.1; 530/350; 435/69.1; 435/320.1; 435/325; 530/399; 530/388.25; 514/12; 514/44; 435/7.1

(57) ABSTRACT

The present invention concerns several splice variants of a Chordin like homologues (CHL2) and depicts their nucleic acid and amino acid sequences, vectors and host cells containing said nucleic acid sequences and antibodies reactive with the amino acid sequences. The invention also concerns pharmaceutical compositions for the treatment of a plurality of diseases, comprising nucleic acid sequences, amino acid sequences, expression vectors and antibodies. The invention also concerns methods for detecting the above nucleic acid or amino acid sequences or antibodies in a biological sample.

### FIELD OF THE INVENTION

The present invention concerns novel nucleic acid sequences, vectors and host cells containing them, amino acid sequences encoded by said sequences, and antibodies reactive with said amino acid sequences, as well as pharmaceutical compositions comprising any of the above. The present invention further concerns methods for screening for candidate activators or deactivators utilizing said amino acid sequences.

### BACKGROUND OF THE INVENTION

The TGF_{β} superfamily is composed of a range of functional and structural factor subclasses with predominantly growth-inhibitory cellular actions and developmental regulatory effects on organogenesis, pattern formation, modulation of extracellular matrix and terminal differentiation. The subclasses include TGF_{β}, activins, glial-derived factors (GDFs), Mulletian inhibiting substances, glial-derived neurotrophic factor (GDNF), cartilage-derived morphogenetic proteins (CDMPs) and the rapidly expanding subclass of bone morphogenic proteins (BMPs). BMPs participate in a broad spectrum of cellular inducing events involving all three germ layers during metazoan development. There are now known to be 7 members of this family (BMPs 1-7); all except BMP1 are members of the TGF-α family. BMP1 has been classified as a novel regulatory protein. The term ‘bone morphogenetic’ may, however, prove to be a misnomer, since the messenger RNAs (mRNAs) for the BMPs are expressed in a wide variety of tissues, suggesting limited tissue or function specificity.

Chordin is an abundant glycoprotein with molecular mass of 120 Kda. It contains internal cysteine rich repeats (CR repeats) called Von Willbrand domains, and N-glycosylation sites.

Chordin is a key developmental protein that dorsalizes early vertebrate embryonic tissues by binding to ventralizing TGF-beta-like bone morphogenetic proteins (BMP) and sequestering them in latent complexes. Chordin binds to ventral BMP-2 and BMP-4 signals in the extracellular space, blocking the interaction of BMPs with their receptors. Chordin mimics the action of the Spemann organizer and can induce the formation of neural tissue from ectoderm and dorsalization of the ventral mesoderm to form muscle.

During early embryogenesis of vertebrates and invertebrates, antagonism between BMPs and several unrelated proteins is a general mechanism by which the dorso-ventral axis is established. One of these extracellular antagonists is Chordin, which binds with high affinity to certain BMPs, preventing their interaction with their cognate cell surface receptors. Chordin plays a role in dorso-ventral axis formation and induction, as well as in maintenance and differentiation of neural tissues in early vertebrate embryogenesis. The inhibitory activity of Chordin on BMPs is mediated by binding through specific domains named Cysteine-Rich (CR) repeats.

Chordin, a secreted protein of 955 amino acids (aa), contains four highly conserved CR repeats. The conservation of each specific CR repeat between Chordin orthologs in different species is higher than that of different CRs within a particular ortholog.

The individual CR repeats in Chordin vary in their binding affinity to BMPs, but they function cooperatively in the full-length protein (Larrain et al., 2000).

Several extracellular proteins contain CR repeats similar to those present in Chordin (Abreu et al., 2002). The similarity is reflected mainly in the spacing of the cysteines within the CR repeat, and in the presence of the CXXCXC and CCXXC motifs. Some of these extracellular proteins, like Chordin itself, contain exclusively CR domains, whereas others also have other protein domains. In addition, the number of CR domains and their location on the protein vary considerably (Abreu et al., 2002).

Several alternatively spliced transcripts have been reported for the human Chordin gene (Millet et al., 2001). These variants were found to be differentially expressed in various tissues, and code for C-truncated isoforms of the Chordin protein that vary in their content of CR repeats and in their biological activity as BMP antagonists (Millet et al., 2001).

A New Chordin-like protein (CHL) was recently reported (Nakayama et al., 2001). CHL also binds and inhibits BMP activity (Nakayama et al., 2001).

Chordin is expressed in fetal and adult tissues (Millet et al., 2001). During embryogenesis and organogenesis, Chordin and CHL display distinct spatiotemporal expression patterns (Nakayama et al., 2001).

Although a single band is observed by northern blot analysis of CHL (Coffinier et al., 2001; Nakayama et al., 2001), several splicing variants of mouse and human CHL have been reported which differ primarily in the length and sequence of their C-termini (Sakuta et al., 2001; Nakayama et al., 2001).

CHL has been shown to be secreted and to bind BMPs and other TGF_{β} superfamily members (Nakayama et al., 2001). Expression patterns as well as functional studies in mouse, chicken and xenopus, indicate that it may function as a modulator of BMP signaling during embryonic development.

Recently, another chordin-like protein, which is structurally most homologous to CHL/neuralin/ventroptin, was identified (Development, 2004 January; 131(1):229-40. Epub 2003 Dec. 03.). When injected into Xenopus embryos, RNA of this protein induced a secondary dorso-ventral axis. Recombinant protein interacted directly with BMPs in a competitive manner to prevent binding to the type I BMP receptor ectodomain, and inhibited BMP-dependent induction of alkaline phosphatase in C2C12 cells. Thus, this protein behaves as a secreted BMP-binding inhibitor. In situ hybridization revealed that expression of this protein is restricted to chondrocytes of various developing joint cartilage surfaces and connective tissues in reproductive organs. Adult mesenchymal progenitor cells expressed this protein, and its levels decreased during chondrogenic differentiation. Addition of this protein to a chondrogenic culture system reduced cartilage matrix deposition. Consistently, protein transcripts were weakly detected in normal adult joint cartilage. However, its expression was upregulated in middle zone chondrocytes in osteoarthritic joint cartilage (where hypertrophic markers are induced). This protein depressed chondrocyte mineralization when added during the hypertrophic differentiation of cultured hyaline cartilage particles. Thus, this protein may play negative roles in the (re)generation and maturation of articular chondrocytes in the hyaline cartilage of both developing and degenerated joints.

A novel member of the Chordin-like protein family was identified and characterized by the present applicant in human and in mouse (PCT Application No. WO 01/34796, hereby incorporated by reference as if fully set forth herein). This novel protein, named CLH, shows high similarity to the recently reported CHL protein (Nakayama et al., 2001), also named Neuralin-1 (Coffinier et al., 2002) or Ventroptin (Sakuta et al., 2001). For the sake of clarity, CLH will be referred to here as CHL2, since it is most closely related to the CHL sequence reported by Nakayama et al.

The high level of homology between CHL2 and CHL is reflected not only in the protein sequence, for example with regard to the number and location of the CR repeats (two adjacent repeats at the N′-terminus, and a third one further downstream), and the absence of other recognizable protein domains, but also in the gene structure, number and size of exons and the spacing of the CR repeats within the exons. Further characterization of CHL2 revealed ubiquitous expression in a variety of tissues and complex alternative splicing, resulting in differentially expressed CHL2 isoforms that differ in their C-termini, the presence of a signal peptide, and the content of their CR repeats.

It has been postulated that Chordin may be expressed by cells of the osteoblast lineage to limit BMP actions in osteoblasts. This may suggest an important function for Chordin as a BMP binding protein since excessive BMP-4 has been implicated in pathogenesis of Fibrodysplasia Ossificans Progressiva (FOP). FOP is a rare genetic disease in which muscles, tendons, ligaments and other connective tissues may ossify into bone. BMPs can cause induction of noggin and Chordin mRNA and protein levels in skeletal cells by transcriptional mechanisms, and these, in turn, prevent the effect of BMPs in osteoblasts in a negative-feedback mechanism. The induction of these proteins by BMPs appears to be a mechanism to limit the BMP effect in bones. Existing therapies which are being investigated for their effectiveness in preventing heterotopic bone formation include inhibitors of BMPs.

Other BMPs have also been linked to pathological conditions. For example, BMP-7 in an animal model can prevent the onset of bone degeneration and associated scarring to the bone marrow that occurs as result of chronic kidney disease. BMP-7 is also linked to inflammatory and malignant processes in the gut, although its role is not completely clear. On the one hand, abundant expression of BMP-7 in the developing intestine was linked to a malignant phenotype and inflammation in the gut. Furthermore, systemically administered BMP-7 against trinitrobenzenesulfonic (TNBS) acid induced inflammatory bowel disease (IBD) in rats leads to a much less severe form of colitis, suggesting that BMP-7 plays an important role in the regulation of anti-inflammatory response in the adult gut tissue.

BMP4 is expressed in ovarian cancer (OC) cells, and long-term BMP4 treatment of primary OC cells results in decreased cell density as well as increased cell spreading and adherence. The BMPR2 (bone morphogenetic protein receptor type 2) mutations have been identified in a substantial portion of patients with familial or sporadic PPH (Primary pulmonary hypertension).

Considerable evidence exists supporting a role for TGF in morphogenesis, in the regulation of endochondral ossification and in bone remodeling. TGF affects proliferation and differentiation of osteoblasts in vitro and high levels of mRNA are expressed in the growth plate of fetal human long bones.

### SUMMARY OF THE INVENTION

The background art does not teach or suggest all chordin-like proteins, which are involved in neuronal development and may also play roles in various types of cancers.

The present invention concerns novel naturally occurring sequences of splice variants of the Chordin-like protein 2 (CHL2), a protein having a significant homology to the Chordin-like protein (CHL) having a nucleotide sequence according to accession number AX175130 (also described in PCT Application No. WO 01/42465, hereby incorporated by reference as fully set forth herein) and to the group of Chordin proteins. The Chordin-like sequences and variants already described in WO 01/34796 will be referred to at times as the “original sequence” (or sequences), or the “native sequences”, and are described in Example XII of the present application, although it should be noted that the term “CHL2” is used both for variants already disclosed in PCT Application No. WO 01/34796 and also those sequences additionally disclosed herein. These new variants of the invention are found both in humans and mice.

The naturally occurring splice variants of the invention differ from known Chordin or known splice variants of Chordin in their unique pattern of exon skipping, creating new unique continuous sequences between the newly ligated exons flanking the skipped exon, or intron inclusion (creating new unique sequences in the expressed mRNA).

The splice variants of the present invention were found to be expressed in a number of specific tissues, and furthermore their level of expression was found to vary significantly between normal and diseased tissue making them suitable markers for disease states.

Thus the present invention provides by its first aspect, the “pro-CHL2” aspect, a novel isolated nucleic acid molecule comprising the sequence of any one of SEQ ID NO: 1 to SEQ ID NO: 5, fragments of said sequences having at least 20 nucleic acids, or a molecule comprising a sequence having at least 80%, preferably 90%, and most preferably 95% or 98% identity to any one of SEQ ID NO:1 to SEQ ID NO: 5.

Thus the present invention provides An isolated nucleic acid sequence selected from:
(i) the nucleic acid sequence comprising in any one of SEQ ID NO: 1 to SEQ ID NO: 5;
(ii) nucleic acid sequences having at least about 80%, homology to the sequence of any one of SEQ ID NO: 1 to SEQ ID NO: 5;
(iii) fragments of (i) or (ii) of at least 20 nucleotides provided that the fragment is not completely identical to a continuous stretch of 20 nucleotides of any of SEQ ID NOs 6-10 or 74-84 or a nucleotide sequence according to accession number AX175130.

Preferably the fragments of (iii) above are selected from the group consisting of:
(i) a fragment being at least about 70%, 80%, 90%, 95% homologous to a portion of SEQ ID NO: 1 corresponding to a bridge between exons 9 and 10, said bridge including at least about 20, 30, 40, 50, 60, 70, 80, 90, 100 nucleotides of exons 9 and 10;
a fragment being at least about 70%, 80%, 90%, 95% homologous to a portion of SEQ ID NO:2 corresponding to a bridge between exons 7 and 9, said bridge including at least about 20, 30, 40, 50, 60, 70, 80, 90, 100 nucleotides of exons 7 and 9;
a fragment being at least about 70%, 80%, 90%, 95% homologous to a portion of SEQ ID NO:3 corresponding to a bridge between exons 2 and 4 or 9 and 10, said bridge including at least about 20, 30, 40, 50, 60, 70, 80, 90, 100 nucleotides of exons 2 and 4 or 9 and 10;
a fragment being at least about 70%, 80%, 90%, 95% homologous to a portion of SEQ ID NO:4 corresponding to a bridge between exons 4 and 6, said bridge including at least about 20, 30, 40, 50, 60, 70, 80, 90, 100 nucleotides of exons 4 and 6;
a fragment being at least about 70%, 80%, 90%, 95% homologous to a portion of SEQ ID NO:5 corresponding to a bridge between exons 2 and 4 or 4 and 6, said bridge including at least about 20, 30, 40, 50, 60, 70, 80, 90, or 100 nucleotides of exons 2 and 4 or 4 and 6.

The present invention further provides a protein or polypeptide comprising or consisting of an amino acid sequence encoded by any of the above nucleic acid sequences, termed herein “CHL2 product”, for example, an amino acid sequence having the sequence in any one of SEQ ID NO: 11 to 15, fragments of the above amino acid sequences having a length of at least 10 amino acids, as well as homologues of the amino acid sequences of any one of SEQ ID NO: 11 to 15 in which one or more of the amino acid residues has been substituted (by conservative or non-conservative substitution) added, deleted, or chemically modified.

Thus the present invention concerns an amino acid sequence selected from:
(i) an amino acid sequence having the sequence of any one of SEQ ID NO: 11 to SEQ ID NO: 15,
(ii) an amino acid sequence having at least about 80%, homology to the sequence of any one of SEQ ID NO: 11 to SEQ ID NO: 15 wherein said amino acid sequence is less than about 90% homologous to any of SEQ ID NOS 16-20 or 85-95, or to a protein having a sequence corresponding to the nucleotide sequence given in accession number AX175130; or
(iii) a portion of a unique region of any of SEQ ID NOS 11-15 selected from:
   a. a bridge portion of SEQ ID NO: 11, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KG, having a structure as follows (numbering according to SEQ ID NO:11): a sequence starting from any of amino acid number 373-x to 373; and ending at any of amino acid numbers 374+((n−2)−x), in which x varies from 0 to n-2;
   b. a bridge portion of SEQ ID NO: 12, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KE, having a structure as follows (numbering according to SEQ ID NO:12): a sequence starting from any of amino acid number 250-x to 250; and ending at any of amino acid numbers 251+((n−2)−x), in which x varies from 0 to n-2;
   c. a bridge portion of SEQ ID NO: 13, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EN, having a structure as follows (numbering according to SEQ ID NO:13): a sequence starting from any of amino acid number 45-x to 45; and ending at any of amino acid numbers 46+((n−2)−x), in which x varies from 0 to n-2; wherein if the peptide is 50 amino acids in length, the starting position cannot be any smaller than 1;
   d. a bridge portion of SEQ ID NO: 14, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TM, having a structure as follows (numbering according to SEQ ID NO:14): a sequence starting from any of amino acid number 124-x to 124 and ending at any of amino acid numbers 125+((n−2)−x), in which x varies from 0 to n-2, wherein the ending position is not greater than 142;
   e. a bridge portion of SEQ ID NO: 15, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EN, having a structure as follows (numbering according to SEQ ID NO:15): a sequence starting from any of amino acid number 45-x to 45; and ending at any of amino acid numbers 46+((n−2)−x), in which x varies from 0 to n-2; wherein if the peptide is 50 amino acids in length, the starting position cannot be any smaller than 1;
(iv) a portion of an amino acid sequence, having at least about 80% homology to a portion of SEQ ID NOs 11-15 defined in (iii) or (v).

The present invention further provides_nucleic acid molecule comprising or consisting of a sequence which encodes the above amino acid sequences, (including the fragments and analogs of the amino acid sequences). Due to the degenerative nature of the genetic code, a plurality of alternative nucleic acid sequences, beyond SEQ ID NO: 1 to SEQ ID NO: 5, can code for the amino acid sequence of the invention. Those alternative nucleic acid sequences which code for the same amino acid sequences encoded by the sequences of SEQ ID NO:1 to SEQ ID NO: 5 are also an aspect of the of the present invention.

The present invention further provides expression vectors and cloning vectors comprising any of the above nucleic acid sequences, as well as host cells transfected by said vectors.

The present invention still further provides pharmaceutical compositions comprising, as an active ingredient, said nucleic acid molecules, said expression vectors, said protein or polypeptide, or antibodies that specifically bind to said protein or polypeptide.

The present invention yet further provides said nucleic acid molecules for diagnosing a disease in which specific hCHL2 variants of the invention are expressed differently than in healthy individuals. Such differences in expression can include over-expression, under-expression, expression in tissues where the gene is usually not expressed in healthy individuals (i.e. spatial mis-expression), and expression in developmental stages wherein the gene is usually not expressed (temporal mis-expression). In one embodiment, the present invention provides a kit comprising said nucleic acid molecules, for diagnosing a disease in which specific hCHL2 variants of the invention are expressed differently than in healthy individuals.

By a second aspect, the “anti-CHL2” aspect, the present invention provides a nucleic acid molecule comprising or consisting of a non-coding sequence which is complementary to that of any one of SEQ ID NO: 1 to SEQ ID NO: 5, or complementary to a sequence having at least 80%, preferably 90%, most preferably 95% or 98% identity to said sequences or a fragment of said sequences. The complementary sequence may be a DNA sequence which hybridizes to any one of the sequences of SEQ ID NO: 1 to SEQ ID NO: 5, or hybridizes to a portion of these sequences which includes the “unique” sequences or bridges (see Glossary), and which has a length sufficient to inhibit the transcription of any one of the sequences of SEQ ID NO: 1 to SEQ ID NO:5. The complementary sequence may be a DNA sequence which can be transcribed into an mRNA being an antisense of the mRNA transcribed from any one of SEQ ID NO: 1 to SEQ ID NO: 5 amend or into an mRNA which is an antisense to a fragment of the mRNA transcribed from any one of SEQ ID NO: 1 to SEQ ID NO: 5 which has a length sufficient to hybridize with the mRNA transcribed from any one of SEQ ID NO: 1 to SEQ ID NO: 5, so as to inhibit its translation. The complementary sequence may also be the mRNA or the fragment of the mRNA itself.

The present invention still further provides pharmaceutical compositions comprising, as an active ingredient, said complementary sequences, or any vectors comprising them. The pharmaceutical compositions of the invention (according to both aspects) may be used for the treatment of a plurality of diseases.

According to still a further aspect of the present invention there is provided a method of diagnosing predisposition to, or presence of a CHL2-related disease in a subject, the method comprising determining a level of a CHL2 polypeptide including at least a portion of an amino acid sequence at least about 70%, preferably at least 80%, more preferably at least 85%, most preferably at least about 90%, even more preferably at least about 95% homologous to any one or more of SEQ ID NOs: 11-15, optionally any one or more of SEQ ID NOs: 16-20 or 85-95, as determined using the BlastP software of the National Center of Biotechnology Information (NCBI) using default parameters.

The method of diagnosis may further involve use of a polynucleotide (mRNA) encoding the polypeptide in a biological sample obtained from the subject, wherein the level of the polynucleotide (mRNA) or the level of the polypeptide is correlatable with predisposition to, or presence or absence of the CHL2-related disease, thereby diagnosing predisposition to, or presence of CHL2-related disease in the subject.

According to still further features in the described preferred embodiments the determining level of the polynucleotide is effected via an assay selected from the group consisting of PCR, RT-PCR, quantitative RT-PCR, chip hybridization, RNase protection, in-situ hybridization, primer extension, Southern blot, Northern blot and dot blot analysis.

By another aspect the present invention concerns a kit for detecting prostate cancer, comprising:
(a) a probe for hybridizing to hCHL2 variant X (SEQ ID NO 10) or fragment thereof, optionally linked to a detectable label;
(b) a calibration curve for comparing hybridization results of a nucleic acid in a sample and the probe, to results obtained with the same probe, from control cells and/or prostate cancer cells.

Typically the kit for detection of prostate cancer of cells comprising:
(a) primers for amplification of h CHL2 (SEQ ID NO 10) or a fragment thereof;
(b) reagents for an amplifications of nucleic acid sequences,

Preferably the fragment is s hCHL2 exon 4a.

By another aspect the present invention concerns a method for detecting prostate cancer, in cells suspected of being cancerous comprising:
(1) determining the level of expression of hCHL2 variant X or a fragment thereof;
(2) comparing the level of (1) to the level of expression in a control healthy cells;
a significantly higher level of expression indication prostate cancer in the cells.

By one embodiment the determination may be by detecting the level of protein depicted by SEQ ID: 20 or a fragment thereof.

By another embodiment the determination of the level of expression is determined by detecting the level of the mRNA of hCHL2 variant X as depicted in SEQ ID NO: 10 or a fragment thereof, preferably using NAT-based technology, preferably wherein fragment comprises hCHL2 exon 4a.

According to preferred embodiments of the present invention, there is provided an antibody or antibody fragment capable of specifically binding to at least one epitope of any of the above-described CHL2 peptides, while distinguishing such peptides from chordin-like sequences that are known in the art. Such “known in the art” sequences preferably include chordin and CHL, and more preferably include any sequence found in PCT Application No. WO 01/34796. A non-limiting illustrative example of an amino acid sequence which an antibody could preferably specifically bind, which would enable the antibody to distinguish between preferred CHL2 peptides of the present invention and the previously described known chordin-like sequences includes the amino acid sequence for the unique exon 2a, found in variants such as Var II for example (see below for more details).

Preferably the antibody of the invention has a selectivity at least two fold higher for an epitope of a sequence of claims 6 to 9 than for an epitope present in any of SEQ ID NOS 16-20 or 85-95, or to a protein having a sequence according to a nucleotide sequence given in accession number AX175130.

According to still a further aspect of the present invention there is provided a kit for diagnosing CHL2-related disease or a predisposition thereto in a subject, the kit comprising the antibody or antibody fragment described above preferably attached to a detectable label and one or more reagents for detecting hybridization of the antibody or antibody fragment.

According to still further features in the described preferred embodiments detecting hybridization of the antibody or antibody fragment is effected by an assay selected from the group consisting of immunohistochemistry, ELISA, RIA, Western blot analysis, FACS analysis, an immunofluorescence assay, and a light emission immunoassay.

According to still further features in the described preferred embodiments, the antibody or antibody fragment is coupled to an enzyme.

According to still further features in the described preferred embodiments the antibody or antibody fragment is coupled to a detectable moiety selected from the group consisting of a chromogenic moiety, a fluorogenic moiety, a radioactive moiety and a light-emitting moiety.

According to still a further aspect of the present invention there is provided a kit for diagnosing CHL2-related disease or a predisposition thereto in a subject, the kit comprising an oligonucleotide as described herein.

According to still further features in the described preferred embodiments the kit further comprising reagents for detecting hybridization of the oligonucleotide.

According to still further features in the described preferred embodiments the CHL2-related disease is selected from the group consisting of bone, uterus, prostate, breast and lung cancers.

The present invention further concerns a biomarker for the detection of d prostate cancer, comprising hCHL2 variant X (SEQ ID No 10) or a fragment thereof preferably exon 4a.

The invention further concerns a primer pair for use in detecting the biomarker of the invention, comprising a primer pair capable of amplifying hCHL2 variant X (SEQ ID 10) or a fragment thereof.
comprising hCHL2 exon 4a-forward primer:

AAACCTCATTTTCTTCTTCCTCCTG (SEQ ID NO: 68.); and hCHL2 exon 4a -Reverse primer:

CTGAAGATCTCTCCGTGTTGGTACATG_(SEQ ID NO:69).

The invention further concerns an amplicon obtained through the use of the above primer pair preferably comprising hCHL2 exon 4a amplicon_(SEQ ID NO:70 . . . ): having the following sequence:

AAACCTCATTTTCTTCTTCCTCCTGCCCCTCCCCCA
CTGCAGAACCTCACACTCCCTCTGGACTCCGGGCCCCACCAAAGTCC
TgCCAGCACAACG GGA CCATGTACCAACACGGA GAGATCTTCAG.

The present invention further concerns a biomarker for detection of cancers comprising the sequence of exon 2a (SEQ ID no:64).

Another biomarker in accordance with the present invention is a biomarker for detecting lung or breast cancer, comprising any one of CHL2 variant IV, V, VI, VII, VIII or IX amino acid sequence according to any of SEQ ID NOs: 13, 14, 15, 17, 18, 19.

The present invention also provides expression vectors comprising any one of the above defined complementary nucleic acid sequences and host cells transfected with said nucleic acid sequences or vectors, being complementary to those specified in the first aspect of the invention.

The invention also provides anti-CHL2 antibodies, namely antibodies directed against the CHL2 product which specifically bind to said CHL2 product at a level that is two fold higher than binding to the parent CHL or known CHL variants (see Glossary for a description). These antibodies are useful both for diagnostic and therapeutic purposes. For example these antibodies may be used as an active ingredient in a pharmaceutical composition as will be explained below.

The pharmaceutical compositions comprising said anti-CHL2 antibodies or the nucleic acid molecule comprising said complementary sequence, are suitable for the treatment of diseases and pathological conditions where a therapeutically beneficial effect may be achieved by neutralizing CHL2 or decreasing the amount of the CHL2 product or blocking its binding to its target, for example, by the neutralizing effect of the antibodies, or as a result of antisense nucleic acids which decrease the expression level of CHL2. Examples of the diseases are any one of those mentioned above.

According to another aspect of the invention the present invention provides methods for detecting the level of the transcript (mRNA) of said CHL2 product in a body fluid sample, or in a specific tissue sample or body fluid, for example, by use of probes comprising or consisting of said coding sequences (or complementary sequences); as well as methods for detecting levels of expression of said product in tissue, e.g. by the use of antibodies capable of specifically reacting with the above amino acid sequences. Detection of the level of the expression of CHL2 of the invention may be indicative of a plurality of physiological or pathological conditions, as detailed above.

According to one aspect of the invention, there is provided a method for detection of a nucleic acid sequence in a biological sample, comprising:
providing a probe comprising at least one of the nucleic acid sequence of the invention defined above;
contacting the biological sample with said probe under conditions allowing hybridization of nucleic acid sequences thereby enabling formation of hybridization complexes;
detecting hybridization complexes, wherein the presence of the complex indicates the presence of nucleic acid sequence encoding the CHL2 product in the biological sample.

The amount of hybridization complexes may be determined and calibrated by comparing it to a calibration scale in order to determine the amount of CHL2-encoding nucleic acid sequence in the sample. The level of each of the sequences may be detected and either compared to the calibration scale or to each other, and said ratio may also be indicative of a plurality of pathological or physiological conditions.

By a preferred embodiment the probe is part of a nucleic acid chip used for detection purposes, i.e. the probe is a part of an array of probes each present in a known location on a solid support.

The nucleic acid sequence used in the above method may be a DNA sequence, an RNA sequence, etc; it may be a coding sequence or a sequence complementary thereto (for detection of RNA transcripts or coding-DNA sequences respectively). By quantifying the level of hybridization complexes and calibrating the quantified results it is possible also to detect the level of the transcript in the sample.

Methods for detecting mutations in the region coding for the CHL2 product are also provided, which may be methods carried out in a binary fashion, namely merely detecting whether there is any mismatches between the normal CHL2 nucleic acid sequence and the one present in the sample, or carried out by specifically detecting the nature and location of the mutation.

The present invention also concerns a method for detecting a protein product in a biological sample, as well as determining its level of expression or changes in said level of expression, the method comprising:
contacting with said biological sample the anti-CHL2 antibody of the invention, thereby forming an antibody-antigen complex; and
detecting said antibody-antigen complex
wherein the presence of said antibody-antigen complex indicates the presence of CHL2 product in said biological sample.

The present invention also concerns a method for detecting antibodies in a biological sample comprising:
contacting said biological sample with the product of the invention thereby forming an antibody-antigen complex; and
detecting said antibody-antigen complex;
wherein the presence of said antibody-antigen complex correlates with the presence of anti-CHL2 antibodies in said biological sample.

In many cases, diseases are detected not by detecting the presence of the protein (product) which caused the disease, but rather by detecting the presence in a biological sample (such as blood or serum) of antibodies against such a product. The method of detecting the presence of anti-CHL2 antibodies is intended to be used in such case.

The amount of the antibody-antigen complex can be quantified, in order to determine the level of the CHL2 product or the anti-CHL2 antibodies, as the case may be. As explained above, the level of any one of the products may be compared to each other, and the ratio between the levels may be indicative of a plurality of physiological and pathological conditions. In addition, the indicative ratio may not be the ratio of the proteins themselves but rather the ratio of antibodies against the proteins.

By yet another aspect the invention also provides a method for identifying candidate compounds or molecules capable of modulating the activity of a protein or polypeptide (being either activators or deactivators), the method comprising:
providing a protein or polypeptide comprising an amino acid sequence as in any one of SEQ ID NO: 11 to SEQ ID NO: 15, or a fragment of such a sequence;
contacting said candidate compound or molecule with said protein or polypeptide;
assessing the biological activity of the protein or polypeptide in the presence and absence of said candidate compound; and
selecting those compounds affect said biological activity.

The biological activity of said protein or polypeptide may be for example the binding of the CHL2 product to various BMPs, the effect of CHL2 on the expression or activity of BMPs, or a therapeutic effect as above. Any compound or molecule which changes such an activity has an potential for serving as an activator or a deactivator.

The present invention also concerns compounds identified by the above methods described above, which compound may either be an activator of the CHL2 product or a deactivator thereof.

General Description of the Invention

The present sequences were discovered by searching a database of expressed sequences (dbEST in NCBI) for putative CR motifs. Two human ESTs that contained part of a CR domain of a previously unknown protein were found. RACE analysis enabled the isolation of a 1.7 Kb cDNA, with an ORF of 1287 nucleotides coding for a novel protein that contains a signal peptide and three CR domains. The first in-frame initiation codon (ATG) was found 295 nucleotides from the 5′ end of this cDNA. No in-frame stop codon was found upstream of this ATG. Support for this ATG being the translation initiation site is supported by the resemblance to Kozak consensus sequences [(A/G)NNatgG] (Kozak, 1996) surrounding this ATG (AGGatgG), as well as the presence of a putative signal peptide immediately downstream from it and the homology to other closely related proteins. Several additional ESTs and partial cDNAs corresponding to this gene and a mouse ortholog were deposited in the GenBank database.

Major transcripts of hCHL2 (corresponding to VarI—SEQ ID NO: 1 and VarII—SEQ ID NO: 6, which differ only in 55 bp and cannot be distinguished by Northern blot assays) were detected in northern blots of fetal, as well as adult tissues, with particularly high levels in the adult uterus. RT-PCR data indicate that hCHL2 transcripts with or without exon 9b are produced in most tissues assayed. The major 2.3 Kb band seen in RNA blots may include both variants, since exon 9b is only 55 bp long. Other alternative variants, such as those excluding exon 8, or initiating in exons 2a or 4a, are not detected in these RNA blots. This may be due to a low level of expression, as suggested also from the weaker intensity of these RT-PCR products and the number of PCR cycles required for their detection.

Further characterization of human and mouse mRNA transcripts by RACE and RT-PCR revealed a complex pattern of alternative splicing, which is described in detail below.

In accordance with the present invention, it has been found that the CHL2 of the invention is located in astrocytes. Astrocytes are known to have a variety of physiological activities in maintaining normal brain physiology, such as in the secretion of active compounds, formation of the blood-brain barrier, metabolism of neurotransmitters and maintenance of the ionic balance of the extracellular space.

Therefore, pharmaceutical compositions in accordance with the present invention may be used to treat diseases and pathological conditions which can be benefited by a modulation of astrocyte activity, such as the modulation of the cross-talk signals in the CNS during physiological and pathological conditions of the nervous system. Examples of such diseases are neuro-degenerative diseases caused by aging, infectious agents, by toxic substances or due to genetic causes, including but not limited to, Parkinson's disease, Alzheimer's disease, neurodegeneration due to age and/or oxidative stress, and diseases having direct genetic causes, including but not limited to ataxia and Huntington's disease. In addition, the pharmaceutical compositions may be used for the treatment of diseases and pathological conditions involving abnormal development of the nervous system, optionally and preferably including or featuring treatment or prophylactic treatment with regard to development of the nervous system after birth.

CHL2 and variants according to the present invention was found by immunohistochemical methods to be localized in fetal-human bone. The antibodies were generated against the coding sequence of Var II, and therefore all the variants that share this coding sequence can be identified using this method, including all of the variants identified by “Var” or “variant” followed by a Roman numeral.

Thus, the pharmaceutical compositions of the present invention may be used for the treatment of diseases and pathological conditions associated with osteoblasts or other diseases of mesanchimal origin. An example of such diseases is FOP, as well as other diseases involving abnormal bone or cartilage formation, metabolism and/or destruction.

Furthermore, the CHL2 variants of the invention were mapped to chromosome 11q14 (genomic clone accession no. APOO 2010; AP001324; ACO118686).

The chromosomal location of the CHL2 gene is near several disorders of cartilage and bone formation, and thus, the pharmaceutical compositions of the invention may be used for the treatment or alleviation of the following diseases:

Osteopetrosis (congenital disorder characterized also by development of abnormally dense bones).

High Bone Mass (HBM)—High bone mass can result from osteosclerosis (increased density of trabecular—spongy bone) and/or hyperostosis (thickening of cortical—compact bone from deposition of osseous tissue) along subperiosteal and/or endosteal surfaces), occurring focally or throughout the skeleton.

The pharmaceutical compositions of the invention may be used also for the treatment of osteoporosis pseudoglioma syndrome, autosomal recessive osteopetrosis, and isolated increased bone mass (high bone mass without other clinical features). The inhibition of the CHL2 of the invention may also be used for augmenting bone regeneration after injury, so as to speed up the healing process.

In accordance with another finding of the present invention, CHL2 of the invention is expressed in the placenta, and is localized in the uterus lining (endometrium). It is known, that poor preparation of the endometrium (uterine lining) has been associated with abnormal pregnancies and a high rate of miscarriages, as well as other disorders of the female reproductive tract. Thus, the pharmaceutical compositions of the invention may be used for the support of a normal pregnancy, as well as for the treatment of abnormal pregnancies, recurring miscarriages, or the malfunction of the female reproductive tract.

Furthermore, the expression of CHL2 of the invention has also been found to be located in the Mullerian epithelia in the internal female ganglia (fallopian tubes, uterus, endocervix glands). CHL2 of the invention can be used to regulate sexual differentiation, for example, by interaction with Mullerian inhibitory substances (MLS), substances secreted by the testes, which causes the regression of the Mullerian duct system in females, leading to female sterility. In addition, CHL2 of the invention may be used for the treatment of the Lawrence-Moon-Bardet-Biedl syndrome, a rare inherited condition with variable manifestations, one of which is hypo-genitalism (underdeveloped genitals).

In accordance with another finding of the invention, CHL2 was found to be expressed in tumors of the uterus, prostate, lung and breast, indicating that CHL2 may be a proliferative agent in cell lines in general and tumor cell lines in particular. Thus, pharmaceutical compositions comprising an agent which can decrease the expression or level of CHL2, such as in anti-sense therapy, or antibodies, may be used for the treatment of these tumors.

CHL2 variants of the present invention, containing the unique exon 4a (e.g. Var X), are differentially expressed in prostate cancer as compared to normal prostate tissue. According to still a further aspect of the present invention there is provided a novel markers for prostate cancer that are both sensitive and accurate. The measurement of these markers, alone or in combination, in patient samples provides information that the diagnostician can correlate with a probable diagnosis of prostate cancer. The markers of the present invention, alone or in combination, show a high degree of differential detection between prostate cancer and non-cancerous states.

The present invention therefore also relates to diagnostic assays for prostate cancer, and methods of use of such markers for detection of prostate cancer (alone or in combination) in a sample taken from a subject (patient). The assays are preferably NAT (nucleic acid amplification technology)-based assays, such as PCR for example (or variations thereof such as real-time PCR for example), but may optionally also feature detection of a protein and/or peptide, for example by using an antibody for such detection. Non-limiting examples of immunoassays encompassed by the present invention include a Western blot assay or an ELISA, although of course other immunoassays could optionally be used. The assays may also optionally encompass nucleic acid hybridization assays. The assays may optionally be qualitative or quantitative.

The present invention also relates to kits based upon such diagnostic methods or assays.

In certain embodiments, the sample taken from the subject can be selected from one or more of seminal plasma, blood, serum, urine, prostatic fluid, seminal fluid, semen, and prostate tissue.

CHL2 variants of the present invention, preferably containing the unique exon 2a (e.g. Var IV, V, VI, VII, VIII, and IX), are differentially expressed in breast cancer and in lung cancer as compared to normal breast and lung tissues. According to still a further aspect of the present invention there is provided a novel markers for breast or lung cancer that are both sensitive and accurate. The measurement of these markers, alone or in combination, in patient samples provides information that the diagnostician can correlate with a probable diagnosis of breast or lung cancer. The markers of the present invention, alone or in combination, show a high degree of differential detection between breast cancer or lung cancer and non-cancerous states.

The present invention therefore also relates to diagnostic assays for breast cancer or lung cancer, and methods of use of such markers for detection of breast cancer or lung cancer (alone or in combination) in a sample taken from a subject (patient). The assays are preferably NAT (nucleic acid amplification technology)-based assays, such as PCR for example (or variations thereof such as real-time PCR for example), but may optionally also feature detection of a protein and/or peptide, for example by using an antibody for such detection. Non-limiting examples of immunoassays encompassed by the present invention include a Western blot assay or an ELISA, although of course other immunoassays could optionally be used. The assays may also optionally encompass nucleic acid hybridization assays. The assays may optionally be qualitative or quantitative.

The present invention also relates to kits based upon such diagnostic methods or assays.

In certain embodiments, the sample taken from the subject can be selected from one or more of blood, serum, urine, sputum, semen or any other bodily fluid or secretion, lung tissue, breast tissue and amniotic fluid.

CHL2 of the invention is a hormone-responsive element, as it is expressed in the Mullerian epithelium, ductal epithelium of the breast and prostate, all of which are tissues under sex hormone control. Thus, since CHL2 is expressed in all estrogen target tissues (and some androgen target tissues), the pharmaceutical compositions of the invention may be used for hormonal regulation in pathological conditions involving abnormal amounts or an abnormal response to sex hormones.

Pharmaceutical compositions of the invention may also be used for the treatment of cardiovascular disorders.

The nucleic acids of the present invention may be used for therapeutic or diagnostic applications, for example, for the detection of the expression of CHL2 in various tissues, as mentioned above (for example, tumors, astrocytes, bone, tissues of the reproductive tract, etc.), and for the detection of any one of its diseases mentioned above. In addition, the relative level of expression of each of the Chordin-like variants (compared to the rest of the variants or to the native sequences) may also be indicative of a plurality of physiological or pathological conditions, for example, any one of the diseases mentioned above.

In accordance with another finding of the invention, CHL2 transcripts were found to be differentially expressed in lung tumor, breast tumor and prostate tumor tissues as compared to normal tissues. Thus the specific CHL2 transcripts can be used as biomarkers for the above diseases. The measurement of these markers in patient samples, alone or in combination, provides information that the diagnostician can correlate with a probable diagnosis of lung, breast or prostate cancer and the amount of these markers may optionally indicate severity/grade of the disease. The term “amount” may optionally include an absolute amount and/or a ratio between an amount found in a normal or non-diseased subject (and/or in one or more tissues of such a subject) and a second found in a non-normal or diseased subject (and/or in one or more tissues of such a subject). It should be noted that the amount in the normal tissue or subject may optionally be higher or lower than the amount in the diseased tissue or subject. The markers of the present invention, alone or in combination, show a high degree of differential detection between cancer and non-cancerous states.

The present invention therefore also relates to diagnostic assays for lung, breast and prostate cancer, and methods of use of such markers for detection of lung, breast and prostate cancer (alone or in combination) in a sample taken from a subject (patient). The assays are preferably NAT (nucleic acid amplification technology)-based assays, such as PCR for example (or variations thereof such as real-time PCR for example), but may optionally also feature detection of a protein and/or peptide, for example by using an antibody for such detection. Non-limiting examples of immunoassays encompassed by the present invention include a Western blot assay or an ELISA, although of course other immunoassays could optionally be used. The assays may also optionally encompass nucleic acid hybridization assays. The assays may optionally be qualitative or quantitative.

The present invention also relates to kits based upon such diagnostic methods or assays.

In certain embodiments, the sample taken from the subject can be selected from one or more of sputum, seminal plasma, blood, serum, urine, prostatic fluid, seminal fluid, semen, and prostate tissue, amniotic fluid (for detection of pre-natal defects).

In one aspect, the invention provides methods for detecting markers which are differentially present in samples from patients with prostate cancer, benign prostate hyperplasia and with negative diagnoses. Any one or combination of markers described are within the scope of this aspect of this invention and can be detected. The methods for detecting these markers have many applications. For example, one marker or combination of markers can be measured to differentiate between prostate cancer and BPH, and thus are useful as an aid in the diagnosis of prostate cancer in a patient. In another example, the present methods for detecting these markers can be applied to in vitro prostate cancer cells or in vivo animal models for prostate cancer to assay for and identify compounds that modulate expression of these markers.

According to other preferred embodiments of the present invention, hCHL2 transcripts containing exon 4a or a fragment thereof comprise a biomarker for detecting prostate cancer. Optionally and more preferably, the fragment of hCHL2 transcripts containing exon 4a comprises unique exon 4a sequence. Also optionally and more preferably, any suitable method may be used for detecting a fragment such as hCHL2 exon 4a for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to hCHL2 transcripts containing exon 4a as described above, including but not limited to SEQ ID NO: 10 (nucleic acid sequence) or 20 (amino acid sequence). Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

According to preferred embodiments of the present invention, the nucleotide and amino acid sequences described below may optionally and preferably have other applications. Furthermore, these nucleotide and amino acid sequences themselves are featured as preferred embodiments of the present invention.

According to preferred embodiments of the present invention, certain novel CHL2 human and mouse proteins are of 429 and 426 aa, respectively, and display high level of homology to the CHL protein discussed above (FIG. 1). These secreted proteins bind to BMPs, and more weakly also to other TGFβ superfamily members. Several studies indicate that CHL/neuralin-1/ventroptin may function as a modulator of BMP signaling during embryonic development. Both CHL and CHL2 present a broad expression pattern in a variety of adult tissues. Expression of the human CHL2 protein (hCHL2) was observed in epithelial cells and in osteoblasts.

The level of similarity and identity among different regions of the CHL and CHL2 human and mouse proteins is summarized in Table 1. The human and mouse CHL2 orthologs are highly conserved, but the level of conservation of human and mouse CHL is even greater. A higher level of conservation is observed within the CR repeats, and is highest within CR3 repeats. The homology between CHL and CHL2 proteins is confined primarily to the CR repeats (Table 1 and FIG. 1).

The conservation of Chordin orthologs among different species is higher for each specific CR than for different CRs within a particular ortholog. This is also true for the conservation of the CRs between the mouse and human orthologs of CHL or CHL2, as shown in Table 1.

TABLE 1
Pairwise comparison between CHL2 and CHL proteins^{a}

| | | Overall | | CR1 | | CR2 | | CR3 | | CR2-CR3^{b} | | CR3-end^{c} | |
|---|---|---|---|---|---|---|---|---|---|---|---|---|---|
| Protein 1 | Protein 2 | I | S | I | S | I | S | I | S | I | S | I | S |
| hCHL2 | mCHL2 | 73 | 77 | 75 | 78 | 78 | 78 | 78 | 81 | 67 | 71 | 75 | 82 |
| hCHL | mCHL | 91 | 92 | 94 | 95 | 97 | 98 | 100 | 100 | 89 | 89 | 86 | 88 |
| hCHL | hCHL2 | 42 | 52 | 57 | 62 | 59 | 66 | 59 | 75 | NS | | 29 | 42 |
| mCHL | mCHL2 | 41 | 49 | 62 | 67 | 56 | 61 | 56 | 65 | NS | | NS | |

I = Identity
S = Similarity
NS = No significant similarity
^{a}Protein sequences were as shown in FIG. 1
^{b}Amino acid sequences between CR2 and CR3
^{c}Amino acid sequences after CR3 up to C-terminus

In the human and mouse genomes, CHL2 genes are composed of 11 exons that span a region of about 35 and 28 kilobases (kb), respectively. The human gene is located on chromosome 11q13.4, and the mouse gene resides on chromosome 7, band 7E1, consistent with the synteny between these chromosomal regions. Syntenic chromosome domains are respective inter-species domains considered to contain orthologous regions. The structure of the human and mouse CHL2 genes, schematically presented in FIG. 2, is very similar, in particular regarding the exon-intron structure, size of exons, and spacing of CR repeats within the exons (FIG. 2). The first exon contains 5′ UTR sequences, the translation initiation codon and a cleavable N-terminal signal peptide at positions 1-25. The two N-terminal CR repeats are encoded each by two exons: exons 2 and 3 encode for CR1, and exons 4 and 5 encode for CR2. CR3 is encoded by one exon, exon 8.

The striking similarity between CHL and CHL2 extends also to the structure of their respective genes, in both the human and mouse genomes, including the size of their exons and the peculiar spacing of the CR repeats (FIG. 2). These observations suggest that CHL and CHL2 resulted by gene duplication.

The highly similar exon-intron structure of the CHL and CHL2 genes suggests that these two paralogues have risen by gene duplication. Indeed, hCHL and hCHL2 are located on ancient duplicated segments of human chromosomes Xq22.1-23 and 1q13.4, respectively.

Like Chordin and CHL, hCHL2 is a secreted protein. His-tagged hCHL2 interacts in vitro with Activin-A, but not with BMP-2, -4 or -6.

Bone Related Diseases:

Osteopetrosis, Autosomal Recessivea

A rare hereditary disease characterized by extreme density and hardness and abnormal fragility of the bones with partial or complete obliteration of the marrow cavities. In this disorder there is a defective resorption of immature bone.

Osteoporosis-Pseudoglioma Syndrome; Oppg

A hereditary disease characterized by abnormally brittle, easily fractured bones, suggesting osteogenesis imperfecta.

High Bone Mass

High Spinal Bone Mineral Density

Osteoarthritis Susceptibility, Female-Specific

Somatotrophinoma, Acromegaly

A chronic disease of adults marked by enlargement of the bones of the extremities, face, and jaw that is caused by overactivity of the pituitary gland.

Nervous System Related Diseases

Pheochromocytoma, Familial Extra adrenal (Also Named Paragangliomas, Hereditary Extra adrenal)

A usually benign tumor of the adrenal medulla or the sympathetic nervous system in which the affected cells secrete increased amounts of epinephrine or norepinephrine. Disorder appears to have been due to a gene on 11q.

Tuberous Sclerosis 4

An inherited disorder of the skin and nervous system that is characterized typically by epilepsy and mental retardation, by a rash of the face resembling acne, and by multiple noncancerous tumors of the brain, kidney, retina, and heart failure, with radiographic evidence of cardiomegaly in all of them. Typical findings of tuberous sclerosis in the central nervous system, kidneys, heart, and liver.

Alexander Disease

This disorder, is characterized clinically by development of megalencephaly in infancy accompanied by progressive spasticity and dementia. In this disorder astrocytes show marked changes.

Hartnup Disorder

This disorder is characterized by a pellagra-like light-sensitive rash, cerebellar ataxia, emotional instability, and aminoaciduria.

Spinal Muscular Atrophy with Respiratory Distress 1

Disorder characterized by the degeneration of motoneurons in the spinal cord, resulting in muscular weakness and atrophy, and that in some forms can be fatal. Neurogenic atrophy of skeletal muscle is observed.

Meckel Syndrome, Type 2; Mks2a

Syndrome inherited as an autosomal recessive trait and typically characterized by occipital encephalocele, microcephaly, cleft palate, polydactyly, and polycystic kidneys.

Schizophrenia Susceptibility Locus, Chromosome 11q-RELATED

Psychotic disorders usually characterized by withdrawal from reality, illogical patterns of thinking, delusions, and hallucinations, and accompanied in varying degrees by other emotional, behavioral, or intellectual disturbances. Schizophrenia, often associated with dopamine imbalances in the brain and defects of the frontal lobe, may have an underlying genetic cause.

Developmental Disorders

Since Chordin play a role in patterning the early embryo development, Chordin-LM might involved in the following disorders:

Ebstein Anomaly

A congenital malformation of the heart that consists of downward placement of the tricuspid valve such that part of the right ventricle becomes incorporated into the pretricuspid chamber. Rearrangements of the long arm of chromosome 11 were described in patients with Ebstein anomaly.

Rutledge Lethal Multiple Congenital Anomaly Syndrome

External features, mesomelic dwarfism, micrognathia, V-shaped upper lip, microglossia, thick alveolar ridges, ambiguous genitalia, webbed neck, highly arched palate, clubfeet, fused fontanelles, inclusion cysts of the tongue, widely spaced nipples, and digital anomalies. Internal findings included oligopapillary renal hypoplasia, severe congenital heart defect, cerebellar hypoplasia, and pulmonary, laryngeal, and gallbladder hypoplasia.

Bardet-Biedl Syndrome, Type 1; Bbs1

The Bardet-Biedl syndrome is characterized by mental retardation, pigmentary retinopathy, polydactyly, obesity, and hypogenitalism. The disorder is inherited as an autosomal recessive.

Targeted inactivation of chordin results in animals that display defects in inner and outer ear development. Therefore chordin-LM might be involved in hearing disorders such as the one linked to chromosome 11-DEAFNESS, Autosomal Dominant Nonsyndromic Sensorineural 11; Dfnal 11.

Glossary

In the following description and claims use will be made, at times, with a variety of terms, and the meaning of such terms as they should be construed in accordance with the invention is as follows:

“Chordin like homolog 2 (CHL2) nucleic acid sequence” (it should be noted that this term is used interchangeably with the term “hCHL2” referring to human variants of chordin like homologs.—the sequence shown in any one of SEQ ID NO: 1 to 5, sequences having at least 80% identity to said sequence and fragments of the above sequences being at least 20 bp long. Those sequences are sequences coding for variants of the homolog produced by alternative splicing. For the purpose of clarity only and without any intention of being limiting, the CHL2 polypeptides encoded by these nucleic acid sequences are described herein as Group I polypeptides, while the nucleic acid sequences themselves are for Group I nucleotide fragments.

The sequences shown in SEQ ID NO: 1 to SEQ ID NO: 5 are all nucleic acid sequences encoding splice variants of the known CHL2 (Chordin—Like) gene.

The first variant (SEQ ID NO: 1, termed “Var T” in the figures) lacks exon 9b (FIG. 8), creating a unique sequence (bridge) between exons 9 and 10.

The second variant (SEQ ID NO: 2, termed “Var III” in the figures) is identical to SEQ ID NO: 1 except that it skips exon 8, and ends with exon 9, creating a unique sequence (bridge) between exons 7 and 9.

The third variant (SEQ ID NO: 3, termed “Var VII” in the figures) starts from exon 2a, skips exon 3 and exon 9b, as described in FIG. 8, creating a unique sequence (bridge) between exon 2 and 4 and another unique sequence (bridge) between 9(a) and 10.

The fourth variant (SEQ ID NO: 4, termed “Var VII” in the figures) starts at exon 2a, skips exon 5 and terminates at exon 9, without exons 9b, 10 and 11, creating a unique sequence (bridge) between exons 4 and 6.

The fifth variant (SEQ ID NO: 5, termed “Var IX” in the figures) is identical to SEQ ID NO: 4, but without exon 3, creating a unique sequence (bridge) between exons 2 and 4, and another unique sequence (bridge) between exons 4 and 6.

It should be noted that the amino acid sequences of the above variants (for which nucleic acid sequences are shown in SEQ ID Nos: 1-5) are preferably described as “consisting essentially of” the numbered sequences; for example, the fifth variant preferably is of a nucleic acid sequence having a sequence consisting essentially of the sequence shown in SEQ ID NO:5.

SEQ IDs NO: 11-15 are the amino acid sequences encoded by SEQ IDs NO: 1-5, respectively.

“Primers and Amplicons According to the Present Invention”

SEQ ID NOs: 21-48 are primers used for PCR amplifications:
a. hCHL2:
SEQ ID NO: 21 is referred to in the figures as p1.
SEQ ID NO: 22 is referred to in the figures as p2.
SEQ ID NO: 23 is referred to in the figures as p3.
SEQ ID NO: 24 is referred to in the figures as p4.
SEQ ID NO: 25 is referred to in the figures as p5.
SEQ ID NO: 26 is referred to in the figures as p6.
SEQ ID NO: 27 is referred to in the figures as p7.
SEQ ID NO: 28 is referred to in the figures as p8.
SEQ ID NO: 29 is referred to in the figures as p9.
b. mCHL2:
SEQ ID NO: 30 is referred to in the figures as p1.
SEQ ID NO: 31 is referred to in the figures as p2.
SEQ ID NO: 32 is referred to in the figures as p3.
SEQ ID NO: 33 is referred to in the figures as p4.
SEQ ID NO: 34 is referred to in the figures as p5.
SEQ ID NO: 35 is referred to in the figures as p6.
c. Human Osteocalcin: SEQ ID NOs: 36 and 37.
d. Mouse Osteocalcin: SEQ ID NOs: 38 and 39.
e. Mouse Myogenin: SEQ ID NOs: 40 and 41.
f. ATP synthase 6: SEQ ID NOs: 42 and 43.
g. 26SPSP: SEQ ID NOs: 44 and 45.
h. Mouse GAPDH: SEQ ID NOs: 46 and 47.
SEQ ID NO 48: mouse CHL2 nucleotide sequence
SEQ ID NO 49: mouse CHL2 protein sequence
SEQ ID NO 50: HPRT1-Forward primer
SEQ ID NO 51: HPRT1-Reverse primer
SEQ ID NO 52: HPRT1 amplicon
SEQ ID NO 53: PBGD-Forward primer
SEQ ID NO 54: PBGD-Reverse primer
SEQ ID NO 55: PBGD amplicon
SEQ ID NO 56: SDHA-Forward primer
SEQ ID NO 57: SDHA-Reverse primer
SEQ ID NO 58: SDHA amplicon
SEQ ID NO 59: G6PD-Forward primer
SEQ ID NO 60: G6PD-Reverse primer
SEQ ID NO 61: G6PD amplicon
SEQ ID NO 62: Exon 2a-Forward primer
SEQ ID NO 63: Exon 2a-Reverse primer
SEQ ID NO 64: amplicon exon 2a
SEQ ID NO 65: Ubiquitin-Forward primer
SEQ ID NO 66: Ubiquitin-Reverse primer
SEQ ID NO 67: Ubiquitin Amplicon
SEQ ID NO 68: Exon 4a Forward primer
SEQ ID NO 69:. Exon 4a-Reverse primer
SEQ ID NO 70: Exon 4a-amplicon
SEQ ID NO 71: RPL-19-Forward primer
SEQ ID NO 72: RPL-19-Reverse primer
SEQ ID NO 73: RPL-19 amplicon

“CLH2 (Chordin Like Homolog) Sequences”

All of the sequences described in this section refer to Group II CLH2 sequences.

SEQ ID NO: 6 (described in the figures as “Var II”) has an accession number of AX140199. Var II contains an additional exon between exons 9 and 10, referred as “9b” in FIG. 8, creating a unique amino acid sequence.

SEQ ID NO: 16 is the amino acid sequence encoded by SEQ ID NO: 6.

SEQ ID NO: 7 (described in the figures as “Var IV”) has an accession number of AX140202. Var IV starts from a unique exon 2a, as is demonstrated in FIG. 8, and contains an additional exon between exons 9 and 10, referred as “9b” in FIG. 8, creating a unique amino acid sequence. SEQ ID NO: 17 is the amino acid sequence encoded by SEQ ID NO: 7.

SEQ ID NO: 8 (described in the figures as “Var V”) has an accession number of AX140203. Var V is identical to Var IV, while it skips exon 8, creating a unique sequence (bridge) between exons 7 and 9. SEQ ID NO: 18 is the amino acid sequence encoded by SEQ ID NO: 8.

SEQ ID NO: 9 (described in the figures as “Var VI”) has an accession number of AX140204. Var VI starts from a unique exon 2a, as is demonstrated in FIG. 8, it skips exon 8, creating a unique sequence (bridge) between exons 7 and 9, and it does not contain exon 9b, creating a unique sequence (bridge) between exons 9 and 10.

SEQ ID NO: 19 is the amino acid sequence encoded by SEQ ID NO: 9.

SEQ ID NO: 10 (described in the figures as “Var X”)has an accession number of AX140201. Var X starts from a unique exon 4a, as is demonstrated in FIG. 8. SEQ ID NO: 20 is the amino acid sequence encoded by SEQ ID NO: 10.

SEQ ID NOS 74-84 are amino acid sequences corresponding to the nucleic acid sequences shown in SEQ ID NOS 74-84, and so form Group II CLH nucleotide fragments. SEQ ID NOS 85-95 form amino acid sequences corresponding to Group II CLH polypeptides.

SEQ ID NO 96: mouse CHL2, corresponding to genbank accession number: AAH19399.

Thus, Group I sequences include amino acid sequences having at least about 70%, optionally at least about 80%, preferably at least about 85%, more preferably at least about 90% and most preferably at least about 95% homology to any of SEQ ID NOs 11-15; and nucleic acid sequences having at least about 70%, optionally at least about 80%, preferably at least about 85%, more preferably at least about 90% and most preferably at least about 95% homology to any of SEQ ID NOs 1-5.

Group II sequences include amino acid sequences having at least about 70%, optionally at least about 80%, preferably at least about 85%, more preferably at least about 90% and most preferably at least about 95% homology to any of SEQ ID NOs 16-20 or 85-95; and nucleic acid sequences having at least about 70%, optionally at least about 80%, preferably at least about 85%, more preferably at least about 90% and most preferably at least about 95% homology to any of SEQ ID NOs 6-10 or 74-84.

In addition, it should be noted that Group I sequences also have unique bridges. These bridges were noted above for the nucleotide sequences in terms of the exons. They are described below in terms of the amino acid sequences, although it should be noted that optionally a nucleotide sequence could be constructed according to any of the amino acid sequences below and used for any purpose ascribed to a nucleotide sequence as described herein. All the alignments were done against Var II, such that the bridges are described with regard to the amino acid sequence of Var II (SEQ ID NO: 16). The bridge is marked on a portion of the actual sequence below by //, which indicates that a portion of the sequence for that SEQ ID NO (relative to the sequence of Var II) is not present.

(SEQ ID NO 11) Variant I Bridge:

RFALEHEASDLVEIYL WKLVK // GIFHLTQIKKV
RKQDFQKEAQHFRLLA

This bridge is present between amino acid positions 373 (lys) and 374 (gly), and preferably comprises a peptide having a sequence taken from either side of these positions. For example, the peptide could optionally comprise a bridge portion of SEQ ID NO: 11, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KG, having a structure as follows (numbering according to SEQ ID NO:11): a sequence starting from any of amino acid number 373-x to 373; and ending at any of amino acid numbers 374+((n−2)−x), in which x varies from 0 to n-2.

For example, for peptides of 10 amino acids (such that n=10), the starting position could be as “early” in the sequence as amino acid number 365 if x=n−2=8 (ie 365=373−8), such that the peptide would end at amino acid number 374 (374+(8−8=0)). On the other hand, the peptide could start at amino acid number 373 if x=0 (ie 373=373−0), and could end at amino acid 382 (374+(8−0=8)).

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: VKGI, KGIF, or LVKG. All peptides feature KG as a portion thereof. Peptides of from about five to about nine amino acids could optionally be similarly constructed.

(SEQ ID NO 12) Variant III Bridge:

PRHFRPKGAGSTTVKIVLKEKHKK//
EDKADPGHSEISSTRCPKAPGRVLV
HTSVSPSPDNLRRFALEHEA

This bridge is present between amino acid positions 250 (lys) and 251 (glu), and preferably comprises a peptide having a sequence taken from either side of these positions. For example, the peptide could optionally comprise a bridge portion of SEQ ID NO: 12, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KE, having a structure as follows (numbering according to SEQ ID NO:12): a sequence starting from any of amino acid number 250-x to 250; and ending at any of amino acid numbers 251+((n−2)−x), in which x varies from 0 to n-2.

The bridge portion above may comprise a peptide being at least 70%, optionally at least about 80%, preferably at least about 85%, more preferably at least about 90% and most preferably at least about 95% homologous to at least one sequence described above.

This variant also has a new N-terminal sequence, which may optionally be constructed as part of a bridge as described above: MALVGLPG.

(SEQ ID NO 14) Variant VIII Bridge:

| | |
|---|---|
| TPSGLRAPPKSCQHNGTMYQHGEIFSAHELFPSRLPNQCVLCSCT | // |
| MRQV SNRMKRTVCSRSMG | |

This bridge is present between amino acid positions 124 (thr) and 125 (met), and preferably comprises a peptide having a sequence taken from either side of these positions. For example, the peptide could optionally comprise a bridge portion of SEQ ID NO: 14, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TM, having a structure as follows (numbering according to SEQ ID NO: 14): a sequence starting from any of amino acid number 124-x to 124 and ending at any of amino acid numbers 125+((n−2)−x), in which x varies from 0 to n-2, wherein the ending position is not greater than 142.

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: CTMR, SCTM, or TMRQ. All peptides feature TM as a portion thereof. Peptides of from about five to about nine amino acids could optionally be similarly constructed.

This variant also has a new N-terminal sequence, which may optionally be constructed as part of a bridge as described above:
MALVGLPG

(SEQ ID NO 15) Variant IX Bridge:

PDMFCLFHGKRYSPGESWHPYLEPQGLMYCLRCTCSE//
NLTLPLDSGPHQSPASTTGPCLFHGKRYSPGESWHPYLEPQGLMYCLRCTCS

This bridge is present between amino acid positions 45 (glu) and 46 (asn), and preferably comprises a peptide having a sequence taken from either side of these positions. For example, the peptide could optionally comprise a bridge portion of SEQ ID NO: 15, comprising a peptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EN, having a structure as follows (numbering according to SEQ ID NO:15): a sequence starting from any of amino acid number 45-x to 45; and ending at any of amino acid numbers 46+((n−2)−x), in which x varies from 0 to n-2; wherein if the peptide is 50 amino acids in length, the starting position cannot be any smaller than 1.

The bridge portion above may comprise a peptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: SENL, ENLT, or CSEN. All peptides feature EN as a portion thereof. Peptides of from about five to about nine amino acids could optionally be similarly constructed.

This variant also has a new N-terminal sequence, which may optionally be constructed as part of a bridge as described above:
MALVGLPG

“Variant”—a sequence produced by alternative splicing of a reference sequence homolog. These sequences are not merely truncated forms of the reference sequence, or modifications of the reference sequence, but rather naturally occurring sequences resulting from various forms of alternative splicing.

“Unique sequence”—as a result of alternative splicing, a non terminal exon is skipped (see for example variant 1 (exon 9b skipped), 2 (exons 9b and 3 are skipped), etc. Skipping of a non-terminal exon creates a unique sequence not present in the parent CHL2 which is the result of a ligation of the two exons flanking the “skipped” exon. This unique sequence results from the unique skipping pattern of the specific variant distinguishing the variant CHL2 of the invention from the parent chordin, or other known variants of chordin. Another possible unique sequence is intron-included sequences marked as exon 2a (variants IV, V, VI, VII, VIII) or exon 4a (variant x). Specific positions of the unique sequences are specified in the claims.

“Identical to the parent Chordin variants”—The present invention optionally and preferably concerns sequences having x% (at least 80%, 85%, 90%, 95%, 98% homology) to any one of the nucleic sequences of SEQ ID NOS 1-5 and/or to the amino acid sequences of SEQ ID NOS 11-15 (Group I sequences). However these homologous sequences are those which are not identical to those of the known parent chordin sequences as shown in Accession Numbers AX175130 or AF209928 (nucleotide sequences; or their corresponding amino acid sequences), or the known chordin variants such as those of (Millet et al., 2001; Nakayama et al., 2001; Sakuta et al., 2001). Optionally, as indicated, these sequences are not identical to those of Group II (nucleic acid sequences of SEQ ID NOs 6-10 or 74-84; or amino acid sequences of SEQ ID NOs 16-20 or 85-95).

“Chordin like homology product (CHL2 product or hCHL2 product)—also referred at times as the “CHL2 protein” or “CHL2 polypeptide”—is preferably a polypeptide according to any Group I sequence, but may optionally (as indicated) also be a polypeptide according to any Group II sequence. The amino acid sequence may be a peptide, a protein, as well as peptides or proteins having chemically modified amino acids (see below) such as a glycopeptide or glycoprotein. An example of a CHL2 product is preferably a polypeptide according to any Group I sequence, but may optionally (as indicated) also be a polypeptide according to any Group II sequence. The term also includes analogues of said sequences in which one or more amino acids has been added, deleted, substituted (see below) or chemically modified (see below) as well as fragments of this sequence having at least 10 amino acids.

“Nucleic acid sequence”—a sequence composed of DNA nucleotides, RNA nucleotides or a combination of both types and may include natural nucleotides, chemically modified nucleotides and synthetic nucleotides.

“Amino acid sequence”—a sequence composed of any one of the 20 naturally appearing amino acids, amino acids which have been chemically modified (see below), or composed of synthetic amino acids.

“Fragment of CHL2 product (hCHL2 product)”—a polypeptide which has an amino acid sequence which is the same as part of but not all of the amino acid sequence of the CHL2 product.

“Fragments of CHL2 (or hCHL2) nucleic acid sequence” a continuous portion, preferably of at least 20 bp of the CHL2 nucleic acid sequence containing the unique sequence where described herein.

“Conservative substitution”—refers to the substitution of an amino acid of one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. [Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of Asp for another Class III residue such as Asn, Gln, or Glu, is a conservative substitution.

“Non-conservative substitution”—refers to the substitution of an amino acid in one class with an amino acid from another class; for example, substitution of Ala, a class II residue, with a class III residue such as Asp, Asn, Glu, or Gln.

“Chemically modified”—when referring to the product of the invention, means a product (protein) where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiquitination, or any similar process or other processes known in the art.

“Biologically active”—refers to the CHL2 products or fragments thereof which have regulatory or biochemical functions on the same target sites which naturally occurring CHL2 influences, for example, which can bind to the same target.

“Immunologically active” defines the capability of a natural, recombinant or synthetic CHL2 product, or any fragment thereof, to induce a specific immune response in appropriate animals or cells and to bind to specific antibodies. Thus, for example, a biologically active fragment of CHL2 product denotes a fragment which retains some or all of the immunological properties of the CHL2 product, e.g can bind specific anti-CHL2 antibodies or which can elicit an immune response which will generate such antibodies or cause proliferation of specific immune cells which produce CHL2-specific antibodies.

“Optimal alignment”—is defined as an alignment giving the highest percent identity score. Such alignment can be performed using a variety of commercially available sequence analysis programs, such as the local alignment program LALIGN using a ktup of 1, default parameters and the default PAM. A preferred alignment is the one performed using the CLUSTAL-W program from MacVector (TM), operated with an open gap penalty of 10.0, an extended gap penalty of 0.1, and a BLOSUM similarity matrix. If a gap needs to be inserted into a first sequence to optimally align it with a second sequence, the percent identity is calculated using only the residues that are paired with a corresponding amino acid residue (i.e., the calculation does not consider residues in the second sequences that are in the “gap” of the first sequence).

“Having at least X% identity”—with respect to two amino acid or nucleic acid sequence sequences, refers to the percentage of residues that are identical in the two sequences when the sequences are optimally aligned. Thus, 80% amino acid sequence identity means that 80% of the amino acids in two or more optimally aligned polypeptide sequences are identical.

“Isolated nucleic acid molecule having an CHL2 nucleic acid sequence”—is a nucleic acid molecule that includes the coding CHL2 nucleic acid sequence. Said isolated nucleic acid molecule may include the CHL2 nucleic acid sequence as an independent insert; may include the CHL2 nucleic acid sequence fused to an additional coding sequences, encoding together a fusion protein in which the CHL2 coding sequence is the dominant coding sequence (for example, the additional coding sequence may code for a signal peptide); the CHL2 nucleic acid sequence may be in combination with non-coding sequences, e.g., introns or control elements, such as promoter and terminator elements or 5′ and/or 3′ untranslated regions, effective for expression of the coding sequence in a suitable host; or may be a vector in which the CHL2 protein coding sequence is a heterologous sequence.

“Expression vector”—refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

“Antibody”—refers to IgG, IgM, IgD, IgA, and IgG antibody. The definition includes polyclonal antibodies or monoclonal antibodies. This term refers to whole antibodies or fragments of the antibodies comprising the antigen-binding domain of the anti-CHL2 product antibodies, e.g. antibodies without the Fc portion, single chain antibodies, fragments consisting of essentially only the variable, antigen-binding domain of the antibody, etc. _The antibodies of the invention have a selectivity of at least two fold higher to the CHL2 of the invention as compared to their binding to known Chordin or known Chordin variants, preferably at least about two fold higher selectivity to Group I sequences as compared to any other sequence described herein, but optionally having at least about two fold higher selectivity to Group II sequences.

The term “substantially retain the antigen binding characteristics of the whole antibody” should be understood to mean that the antibody fragment, derivative or the recombinant antibody molecule specifically binds the CHL2 product and that the affinity for the CHL2 product as determined by Scatchard analysis is at least 30% of the binding affinity of the whole antibody (from which the fragment, derivative or recombinant antibody molecule was derived). In preferred embodiments, the binding affinity of the antibody fragment, derivative or recombinant antibody molecule for the CHL2 product is at least 50% of the binding affinity of the whole antibody.

“Specifically directed against” or “specifically binds” to the CHL2 product means that the antibody recognizes and binds to a particular CHL2 product in preference to other members of the CHL family. Generally, the affinity of anti-CHL2 antibodies is at least 2-fold greater for binding to the CHL2 than for binding to CHL. In a preferred embodiment, the binding affinity of anti-CHL2 antibodies is at least 10-fold greater for binding to the CHL2 than for binding to CHL. The binding affinity may be determined by a Scatchard analysis, a method that is well known in the art.

Specific binding may also be inferred from biological assays, such as therapeutic effects, immunofluorescent studies, binding-competition assays and the like. In such assays, an antibody would be defined as CHL2 specific if the results using this antibody in the experimental setting differ in a statistically significant manner from those of the control settings.

“Activator”—as used herein, refers to a molecule which minics the effect of the natural CHL product or at times even increases or prolongs the duration of the biological activity of said product, as compared to that induced by the natural product. The mechanism may be by binding to the same receptor or target moieties to which native CHL2 binds thus mimicking the activity of CHL2; by prolonging the lifetime of the CHL2, (for example by decrease of the rate of its degradation or clearance) by increasing the activity of CHL2 on its target (modulation of expression and amount of BMPs), by increasing the affinity of CHL2 to moieties to which it binds (such as BMPs) etc. Activators may be small organic molecules, polypeptides, nucleic acids, carbohydrates, lipids, or derivatives thereof, or any other molecules which can bind to and activate the CHL2 product.

“Deactivator” or (“Inhibitor”)—refers to a molecule which modulates the activity of the CHL2 product in an opposite manner to that of the activator, by decreasing or shortening the duration of the biological activity of the CHL2 product. This may be done by blocking the binding of CHL2 to its receptor (competitive or non-competitive inhibition), by causing rapid degradation or clearance of CHL2, etc. by inhibiting association of CHL2 with the effectors which regulate the expression of BMPs, etc. Deactivators may be small organic molecules, polypeptides, nucleic acids, carbohydrates, lipids, or derivatives thereof, or any other molecules which bind to and modulate the activity of said product.

“Treating a disease”—refers to administering a therapeutic substance effective to prevent or ameliorate at least one symptom associated with a disease, to lessen the severity or cure the disease, or to prevent the disease from occurring. Treatment may also refer to slowing down the progression of the disease or the deterioration of the symptoms associated therewith, to enhancing the onset of the remission, disease-free period, to slowing down the irreversible damage caused in the progressive chronic stage of the disease, to delaying the onset of said progressive stage, to improving survival rate or more rapid recovery, to improving life quality of the patient (as evident by pain-free periods, better function, etc.) or a combination of two or more of the above.

The treatment regimen will depend on the type of disease to be treated and may be determined by various considerations known to those skilled in the art of medicine, e.g. the physicians.

“Detection” or “diagnosis”—refer to a method of detection of a disease. This term may also refer to detection of a predisposition to a disease, or to determination of the severity (for example cancer grade) of a known disease, or its prognosis.

“Probe”—the CHL2 nucleic acid sequence, or a sequence complementary therewith, including bridge sequences and sequences complementary therewith, when used to detect presence of other similar sequences in a sample. The detection is carried out by identification of hybridization complexes between the probe and the assayed sequence. The probe may be attached to a solid support or to a detectable label.

The term “marker” in the context of the present invention refers to a nucleic acid fragment, a peptide, or a polypeptide, which is differentially present in a sample taken from patients having prostate cancer as compared to a comparable sample taken from subjects who do not have prostate cancer.

The phrase “differentially present” refers to differences in the quantity of a marker present in a sample taken from patients having the disease to be detected as compared to a comparable sample taken from healthy controls. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

The term “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the term “diagnosing” refers to classifying a disease or a symptom, determining a severity of the disease, monitoring disease progression, forecasting an outcome of a disease and/or prospects of recovery.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease, or to its severity.

As used herein, the term “level” refers to expression levels of RNA and/or protein and/or anti-CHL2 antibody and/or antibody-antigen complexes or to DNA copy number of a marker of the present invention. The present invention preferably encompasses antibodies capable of selectively binding (with at least two fold higher binding) to at least one epitope of a Group I polypeptide as compared to any other polypeptide described herein (optionally including Group II polypeptides). Optionally the present invention preferably encompasses antibodies capable of selectively binding (with at least two higher binding) to at least one epitope of a Group II polypeptide. The present invention also preferably encompasses any antibody-antigen complex formed with such antibodies and epitopes. Optionally and preferably, an epitope comprises a bridge of an amino acid sequence as described in the glossary.

Typically the level of the marker in a biological sample obtained from the subject is different (i.e., increased or decreased) from the level of the same variant in a similar sample obtained from a healthy individual.

As used herein “a biological sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, milk, blood cells, tumors, neuronal tissue, organs, and also samples of in vivo cell culture constituents, amniotic fluid. For example, tissue would optionally and preferably include prostate tissue and/or other tissues of the male genitalia, or reproductive or urinary tracts. A fluid sample would optionally and preferably include blood (optionally including whole blood and/or blood fractions), semen or urine, for example.

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy).

Regardless of the procedure employed, once a biopsy is obtained the level of the variant can be determined and a diagnosis can thus be made.

Determining the level of the same variant in normal tissues of the same origin is preferably effected along-side to detect an elevated expression and/or amplification.

A “test amount” of a marker refers to an amount of a marker present in a sample being tested. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a prostate cancer patient, a BPH patient or a person without prostate cancer or BPH. A control amount can be either in absolute amount (e.g., microgram/ml) or a relative amount (e.g., relative intensity of signals).

### DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a comparison of the human and mouse CHL2 variant I and CHL proteins. Amino acid sequence alignment of the orthologous and paralogous proteins indicates high conservation between these two vertebrate genes. The position of the signal peptide (SP) and the three CR repeats (CR1-CR3) is indicated. Sequences were aligned using the ClustalW program. Identical and similar residues are indicated by dark and light shading, respectively. Dashes indicate gaps introduced to align sequences. Protein sequences taken for the analysis were: hCHL2 (SEQ ID NO:1 1), mCHL2 (SEQ ID NO:96), hCHL (amino acid sequence corresponding to nucleotide sequence given in Genbank accession number AX175130), and mCHL (genebank accession number BC066832).

FIG. 2 shows a schematic representation of the human and mouse CHL2 and CHL genes (sequence identification numbers as for FIG. 1). Shown is the intron-exon genomic organization of the genes. Exons are depicted as boxes, and their size is given in bp. Introns, not drawn to scale, are drawn as thin lines. Coding and untranslated sequences are shown in gray and white, respectively. Sequences encoding for the signal peptide and the CR repeats are indicated on top. Note that CR1 and CR2 are each encoded by two exons, while CR3 is encoded by a single exon.

FIG. 3 shows a northern blot analysis of hCHL2 expression. The probes used can detect several variants: primers p9 (SEQ ID NO: 29)+p8 (SEQ ID NO: 28) amplifying the probe used for hybridization in (A) spans sequences of exons 8-11, and can detect any variant accordingly; primers p1 (SEQ ID NO:21)+p8 (SEQ ID NO: 28) amplifying the probe used for hybridization in (B) spans sequences of exons 1-11, and can detect any variant accordingly. The position of the primers is demonstrated in FIG. 8. The respective sequences are listed as well. Multiple tissue northern blots containing poly(A) RNA of human adult tissues (A) or human fetal tissues (B) were hybridized with a probe derived from the coding sequence of hCHL2. A major transcript of about 2.4 Kb is detected.

FIG. 4 shows an ectopic expression and secretion of hCHL2. COS-7 cells were transfected with hCHL2 variant II (SEQ ID NO: 6) cloned in pcDNA3, or with vector alone. Mock transfected cells served as an additional control. Culture media were collected after 48 and 72 hrs, and the cells were harvested and lysed. Culture media of and cell lysates were fractionated by SDS-PAGE and analyzed by Western blot using rabbit anti-hCHL2. Equal loading of the cell lysates is evident from the intensity of the nonspecific bands. A protein band of 55 kDa is clearly evident only in the culture media and cell lysates of COS-7 cells transfected with the hCHL2-expressing construct.

FIG. 5 shows immunohistochemistry of human tissue sections using polyclonal rabbit anti-hCHL2 (raised against the protein of SEQ ID NO: 16). Preimmune serum served as negative control. Staining of epithelial cells is seen in all tissues analyzed. Staining of osteoblasts in a section of bone from fetal thigh is indicated by an arrow. The counterstain was done using hematoxylin.

FIG. 6 shows a direct interaction of hCHL2 (SEQ ID NO:16) with Activin A. His-tagged hCHL2, secreted to culture media of COS-7 cells transfected with pcDNA4-hCHL2, was immobilized on Talon Metal Affinity Resin, followed by incubation with Activin A. Binding was visualized by immunoblot analysis with anti-Activin goat polyclonal antibody (top panel). Lane 1 contains proteins bound to resin-immobilized His-hCHL2. Lane 2 is same as lane 1, but using culture media from cells transfected with empty vector, as a control for non-specific binding. Lane 3 contains Activin A, as a positive control for the immunoblot. The presence of hCHL2 was subsequently verified on the same membrane, using our anti-hCHL2 rabbit polyclonal antibody (lower panel).

FIG. 7 shows alternative splicing of hCHL2 (the variants are listed in the figure. Primers p1 (SEQ ID NO:21) +p4 (SEQ ID NO: 24) were used to detect variants I, II, III; primers p1 (SEQ ID NO:21)+p8 (SEQ ID NO: 28) were used to detect variants I, II; primers p2 (SEQ ID NO: 22)+p4 (SEQ ID NO: 24) were used to detect variants IV, V, VI, VII, VIII, IX; primers p3 (SEQ ID NO: 23)+p4 (SEQ ID NO: 24) were used to detect variant X; primers p2 (SEQ ID NO: 22)+p7 (SEQ ID NO: 27) were used to detect variants IV, VIII; primers p5 (SEQ ID NO: 25)+p7 (SEQ ID NO: 27) were used to detect variants containing exon 8; primers p1 (SEQ ID NO:21)+p6 (SEQ ID NO: 26) were used to detect variant III) in adult human tissues. Total RNA was used for RT-PCR analysis using several combinations of primers. The structure of the various hCHL2 splice variants identified, as well as the location of the primers employed, are shown in FIG. 8. The RT reactions were standardized with primers for the house keeping gene ATP synthase 6.

FIG. 8 shows alternative splicing of the hCHL2 gene. The exon-intron organization and the primers employed in the RT-PCR analysis are indicated on the top diagram, which shows the entire gene. The various splice variants identified are shown. UTRs are depicted in white, and the ORFs of the splice variants encoding different isoforms are indicated in gray or varying patterns. The size of the protein isoforms is given in amino acids, and the existence of a signal peptide (SP) and the CR repeats is indicated for each isoform.

FIG. 9 shows differential expression of hCHL2 (the exons tested are indicated in the figure, the primers used are indicated as well). Exon 1 (p1 (SEQ ID NO:21)+p4 (SEQ ID NO: 24)) characterizes variants I, II and III; exon 2a (p2 (SEQ ID NO: 22)+p4 (SEQ ID NO: 24)) characterizes variants IV, V, VI, VII, VIII, IX; exon 4a (p3 (SEQ ID NO: 23)+p7 (SEQ ID NO: 27)) characterizes variant X; exon 8 (p5 (SEQ ID NO: 25)+p7 (SEQ ID NO: 27)) characterizes variants I, II, IV, VII, VIII, IX, X) splice variants following PTH-induced osteoblast differentiation. MG-63 and SaOS-2 cells were incubated with PTH (100 nM) for the indicated time periods (where U designates untreated samples). Total RNA was isolated and used for RT-PCR analysis of splice variants that initiated in exons 1, 2a or 4a, and that contain the alternative exon 8. The primers employed are indicated in parenthesis and their location is shown in FIG. 8. Expression of osteocalcin, a marker of osteoblast differentiation, was analyzed to evaluate the cells' response to PTH. The RT reaction products were standardized with primers for the housekeeping gene 26SPSP (26S proteasome subunit p97).

FIG. 10 shows differential expression of mCHL2 genbank accession number: AAH19399 [SEQ ID NO 96] variants following induction of myogenic and osteoblastic differentiation. The exon-intron organization of the mCHL2 gene is shown in the top diagram. C2C12 cells were cultured with 10 μg/ml insulin or300 ng/ml BMP-2 for the indicated time periods. Total RNA was isolated and used for RT-PCR analysis of splice variants that initiate in exons 1a or 1b, and for the absence or presence of exon 8. The primers employed are indicated in parenthesis and their location is shown on the top diagram. Expression of osteocalcin and myogenin was analyzed as markers for osteoblast and myoblast differentiation, respectively. The RT reactions were standardized with the housekeeping gene GAPDH.

FIG. 11 shows Western blot analysis of hCHL2 in A. prostate, endometrium, bone tumor and testis; B. brain and secreted hCHL2 protein. The antibody chosen was raised against Var II as previously described.

FIG. 12 is a histogram showing the relative expression of hCHL2 transcripts containing the amplicon of the unique exon 2a, SEQ ID NO: 64 (e.g., variant no. IV, V, VI, VII, VIII, IX), in normal and cancerous breast tissues as determined by real time PCR using primers for SEQ ID NO: 64 (SEQ ID NO: 62, 63). Expression was normalized to the averaged expression of four housekeeping genes PBGD (GenBank Accession No. BC019323; amplicon—SEQ ID NO: 55, primers SEQ ID Nos: 53, 54), HPRT1 (GenBank Accession No. NM_{—}000194; amplicon—SEQ ID NO: 52, primers SEQ ID Nos: 50, 51), G-6_PD (GenBank Accession No. NM_{—}000402; amplicon—SEQ ID NO: 61, primers SEQ ID Nos: 59, 60) and SDHA (GenBank Accession No. NM_{—}004168; amplicon—SEQ ID NO: 58, primers SEQ ID Nos: 56, 58).

FIG. 13 is a histogram showing the relative expression of hCHL2 transcripts containing the amplicon of the unique exon 2a, SEQ ID NO: 64 (e.g., variant no. IV, V, VI, VII, VIII, IX), in normal and cancerous lung tissues as determined by real time PCR using primers for SEQ ID NO: 64 (SEQ ID NO: 62, 63). Expression was normalized to the averaged expression of four housekeeping genes PBGD (GenBank Accession No. BC019323; amplicon—SEQ ID NO: 55, primers SEQ ID Nos: 53, 54), HPRT1 (GenBank Accession No. NM_{—}000194; amplicon—SEQ ID NO: 52, primers SEQ ID Nos: 50, 51), Ubiquitin (GenBank Accession No. BC000449; amplicon—SEQ ID NO: 67, primers SEQ ID Nos: 65, 66) and SDHA (GenBank Accession No. NM_{—}004168; amplicon—SEQ ID NO: 58, primers SEQ ID Nos: 56, 57).

FIG. 14 is a histogram showing the relative expression of hCHL2 transcripts containing the amplicon of the unique exon 4a, SEQ ID NO: 70, (e.g., variant no. X) in normal, benign and cancerous prostate tissues as determined by real time PCR using primers for SEQ ID NO: 70 (SEQ ID NO: 68, 69). Expression was normalized to the averaged expression of four housekeeping genes PBGD (GenBank Accession No. BC019323; amplicon—SEQ ID NO: 55, primers SEQ ID Nos: 53, 54), HPRT1 (GenBank Accession No. NM_{—}000194; amplicon—SEQ ID NO: 52, primers SEQ ID Nos: 50, 51), RPL19 (GenBank Accession No. NM_{—}000981; amplicon—SEQ ID NO: 73, primers SEQ ID Nos: 71, 72) and SDHA (GenBank Accession No. NM_{—}004168; amplicon—SEQ ID NO: 58; primers SEQ ID Nos: 56, 57).

FIG. 15 is alignment of the CHL2 product of SEQ ID NO: 85 to known chordin protein, demonstrating the homology regions within these proteins. The alignment was performed using best-fit of GCG;

FIG. 16a is the alignment of the first splice variant (SEQ ID NO: 86) to the known chordin deposited in the Emb as gi 4808227;

FIG. 16b is the alignment of the said splice variant (SEQ ID NO: 86) to the known chordin deposited in the Emb under gi 3822218;

FIG. 16c is the alignment of the said splice variant (SEQ ID NO: 86) to the known chordin deposited in the Emb under gi 3800772;

FIG. 17a is the alignment of the second splice variant (SEQ ID NO: 87) with a known chordin deposited in the Emb under gi 4808227;

FIG. 17b is the alignment of the second splice variant (SEQ ID NO: 87) with a known chordin deposited in the Emb under gi 3822218;

FIG. 18a is the alignment of the third splice variant (SEQ ID NO: 88) with a known chordin deposited in the Emb under gi 4808227;

FIG. 18b is the alignment of the third splice variant (SEQ ID NO: 88) with a known chordin deposited in the Emb under gi 2731578;

FIG. 18c is the alignment of the third splice variant (SEQ ID NO: 89) with a known chordin deposited in the Emb under gi 2498235;

FIG. 18d is the alignment of the third splice variant (SEQ ID NO: 89) with a known chordin deposited in the Emb under gi 3822218;

FIG. 19 is multiple alignments of the sequences of the first four splice variants (SEQ ID NOs: 85-89) to several known chordins;.

FIG. 20 is the alignment of SEQ ID No. 89 to the known chordin deposited as gi 48082227;

FIG. 21 is the alignment of SEQ ID No. 89 to the known chordin deposited as gi 3822218;

FIG. 22 is the alignment of SEQ ID No. 89 to the known chordin deposited as gi 6753418;

FIG. 23 shows the alignment of SEQ ID No.90 to the known chording deposited as gi 4808227;

FIG. 24 is the alignment of SEQ ID No.90 to the known chordin deposited as gi 3822218;

FIG. 25 shows the alignment of SEQ ID No.91 to the known chordin deposited as gi 4808222;

FIG. 26 shows the alignment of SEQ ID No.91 to the known chordin deposited as gi 3822218;

FIG. 27 shows the alignment of SEQ ID No. 92 to the known chordin deposited as gi 2731578;

FIG. 28 shows the alignment of SEQ ID No. 92 to the known chordin deposited as gi 3822218;

FIG. 29 shows the alignment of SEQ ID No. 93 to the known chordin deposited as gi 2731578;

FIG. 30 shows the alignment of SEQ ID No. 93 to the known chordin deposited as gi 382218;

FIG. 31 shows the alignment of SEQ ID No. 94 to the known chordin deposited as gi 2731578;

FIG. 32 shows the alignment of SEQ ID No. 94 to the known chordin deposited as gi 3822218.

FIG. 33 shows multiple alignments of SEQ ID Nos. 85-94 (termed var 1-var 6, respectively) to each other;

FIG. 34 shows the alignment of SEQ ID No.95 to the known chordin deposited as gi 480827;

FIG. 35 shows the alignment of SEQ ID No.95 to the known chordin deposited as gi 6753418.

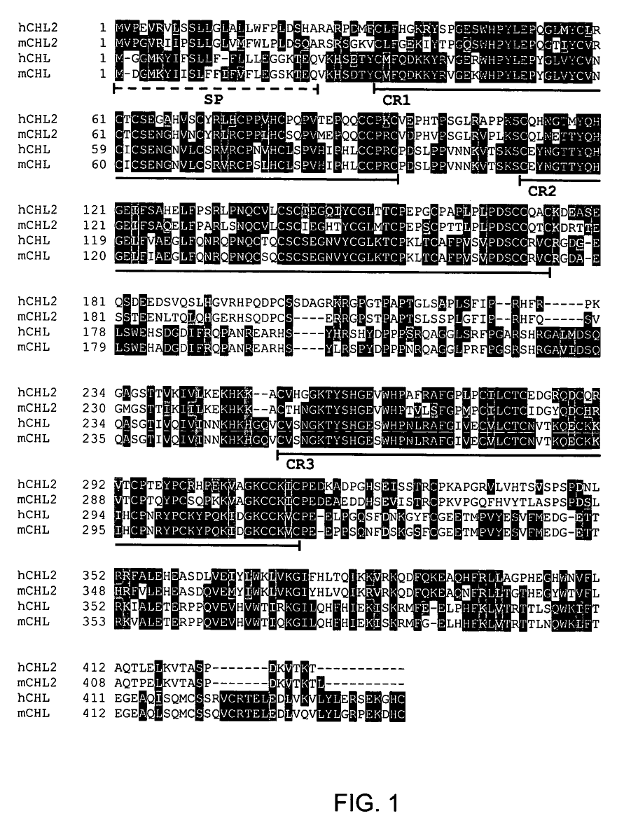

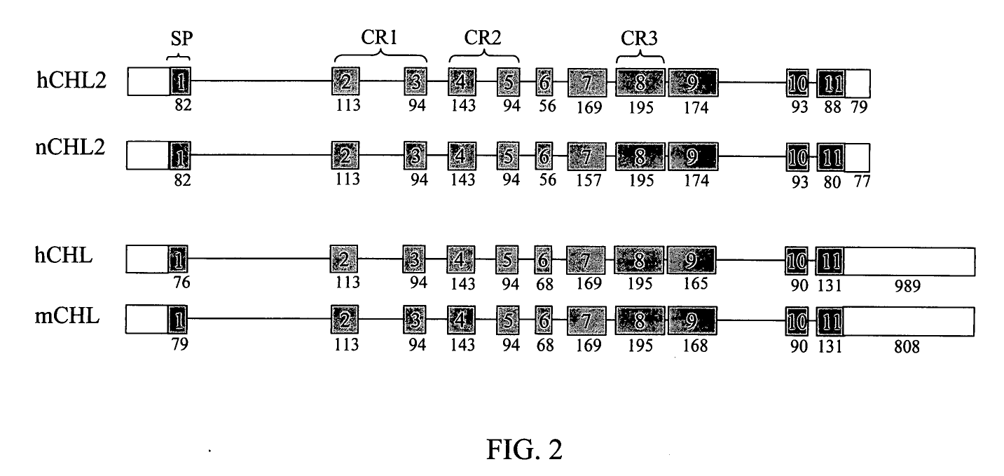

### DETAILED DESCRIPTION OF A PREFERRED EMBODIMENT

### EXAMPLE I

### CHL2—Nucleic Acid Sequence

The nucleic acid sequences of the invention include nucleic acid sequences which encode CHL2 product and fragments and analogs thereof. The nucleic acid sequences may alternatively be sequences complementary to the above coding sequence, or to a region of said coding sequence. The length of the complementary sequence is sufficient to avoid the expression of the coding sequence and should be complementary to a sequence of at least 10 continuous nucleotides not present as a continuous stretch in known CHLs, i.e at least 10 nucleotides of the unique sequence. The nucleic acid sequences may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand. The nucleic acid sequences may also both include dNTPs, rNTPs as well as non naturally occurring sequences. The sequence may also be a part of a hybrid between an amino acid sequence and a nucleic acid sequence.

In a general embodiment, the nucleic acid sequence has at least about 80%, preferably at least about 90% or at least about 95% or at least about 98% sequence identity with any one of the sequences identified as SEQ ID NO: 1 to SEQ ID NO: 5, or alternatively having such sequence identity with any one of the sequences identified as SEQ ID NOS: 6-10 or 74-84.

The nucleic acid sequences may include the coding sequence by itself. By another alternative the coding region may be in combination with additional coding sequences, such as those coding for fusion protein or signal peptides, in combination with non-coding sequences, such as introns and control elements, promoter and terminator elements or 5′ and/or 3′ untranslated regions, effective for expression of the coding sequence in a suitable host, and/or in a vector or host environment in which the CHL2 nucleic acid sequence is introduced as a heterologous sequence.

The nucleic acid sequences of the present invention may also have the product coding sequence fused in-frame to a marker sequence which allows purification of the CHL2 product. The marker sequence may be, for example, a hexahistidine tag to allow purification of the mature polypeptide fused to the marker in the case of a bacterial host, or, the marker sequence may be a hemagglutinin (HA) tag when a mammalian host, e.g. COS-7 cells, is used. The HA tag corresponds to an epitope derived from the influenza hemagglutinin protein (Wilson, I., et al. Cell 37:767 (1984)).

Also included in the scope of the invention are fragments also referred to herein as oligonucleotides, typically having at least 20 bases, preferably from about 20 to about 30 bases corresponding to a region of the coding- sequence nucleic acid sequence. The fragments may be used as probes, primers, and when complementary also as antisense agents, and the like, according to known methods.

As indicated above, the nucleic acid sequence may be as in any one of SEQ ID NO: 1 to SEQ ID NO: 5 or fragments thereof or'sequences having at least about 80%, preferably at least about 90-95%, most preferably at least about 95% or 98% identity to the above sequence, and which are Group I sequences; or alternatively, at least about 80%, preferably at least about 90-95%, most preferably at least about 95% or 98% identity to any of SEQ ID NOS 6-10 or 74-84, which are Group II sequences. Alternatively, due to the degenerative nature of the genetic code, the sequence may be a sequence coding the amino acid sequence of any one of SEQ ID NO: 11 to SEQ ID NO: 15, or fragments or analogs of said amino acid sequence, which is preferably an isolated cDNA or mRNA sequence, which are Group I sequences; or alternatively any of SEQ ID NOS: 16-20 or 85-95, which are Group II sequences; or for either group, homologous sequences thereof as described herein.

A. Preparation of Nucleic Acid Sequences

The nucleic acid sequences may be obtained by screening cDNA libraries using oligonucleotide probes which can hybridize to or PCR-amplify nucleic acid sequences which encode the CHL2 products disclosed above. cDNA libraries prepared from a variety of tissues are commercially available and procedures for screening and isolating cDNA clones are well-known to those of skill in the art. Such techniques are described in, for example, Sambrook et al. (1989) Molecular Cloning: A Laboratory Manual (2nd Edition), Cold Spring Harbor Press, Plainview, N.Y. and Ausubel F M et al. (1989) Current Protocols in Molecular Biology, John Wiley & Sons, New York, N.Y.

The nucleic acid sequences may be extended to obtain upstream and downstream sequences such as promoters, regulatory elements, and 5′ and 3′ untranslated regions (UTRs). Extension of the available transcript sequence may be performed by numerous methods known to those of skill in the art, such as PCR or primer extension (Sambrook et al., supra), or by the RACE method using, for example, the Marathon RACE kit (Clontech, Cat. #K 1802-1).

Alternatively, the technique of “restriction-site” PCR (Gobinda et al. PCR Methods Applic. 2:318-22, (1993)), which uses universal primers to retrieve flanking sequence adjacent a known locus, may be employed. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia, T. et al., Nucleic Acids Res. 16:8186, (1988)). The primers may be designed using OLIGO(R) 4.06 Primer Analysis Software (1992; National Biosciences Inc, Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Capture PCR (Lagerstrom, M. et al., PCR Methods Applic. 1:111-19, (1991)) is a method for PCR amplification of DNA fragments adjacent to a known sequence in human and yeast artificial chromosome DNA. Capture PCR also requires multiple restriction enzyme digestions and ligations to place an engineered double-stranded sequence into a flanking part of the DNA molecule before PCR.

Another method which may be used to retrieve flanking sequences is that of Parker, J. D., et al., Nucleic Acids Res., 19:3055-60, (1991)). Additionally, one can use PCR, nested primers and PromoterFinder™ libraries to “walk in” genomic DNA (PromoterFinder™; Clontech, Palo Alto, Calif.). This process avoids the need to screen libraries and is useful in finding intron/exon junctions. Preferred libraries for screening for full length cDNAs are ones that have been size-selected to include larger cDNAs. Also, random primed libraries are preferred in that they will contain more sequences which contain the 5′ and upstream regions of genes.

A randomly primed library may be particularly useful if an oligo d(T) library does not yield a fill-length cDNA. Genomic libraries are useful for extension into the 5′ untranslated regulatory region.

The nucleic acid sequences and oligonucleotides of the invention can also be prepared by solid-phase methods, according to known synthetic methods. Typically, fragments of up to about 100 bases are individually synthesized, then joined to form continuous sequences up to several hundred bases.

B. Use of CHL2 Nucleic Acid Sequence for the Production of CHL2 Products

In accordance with the present invention, nucleic acid sequences specified above may be used as recombinant DNA molecules that direct the expression of CHL2 products.

As will be understood by those of skill in the art, it may be advantageous to produce CHL2 product-encoding nucleotide sequences possessing codons other than those which appear in any one of SEQ ID NO: 1 to SEQ ID NO: 5 (optionally in any one of SEQ ID NOS 6-10 or 74-84) which are those which naturally occur in the human genome. Codons preferred by a particular prokaryotic or eukaryotic host (Murray, E. et al. Nuc Acids Res., 17:477-508, (1989)) can be selected, for example, to increase the rate of CHL2 product expression or to produce recombinant RNA transcripts having desirable properties, such as a longer half-life, than transcripts produced from naturally occurring sequence.

The nucleic acid sequences of the present invention can be engineered in order to alter a CHL2 product coding sequence for a variety of reasons, including but not limited to, alterations which modify the cloning, processing and/or expression of the product. For example, alterations may be introduced using techniques which are well known in the art, e.g., site-directed mutagenesis, to insert new restriction sites, to alter glycosylation patterns, to change codon preference, to produce splice variants, etc.

The present invention also includes recombinant constructs comprising one or more of the sequences as broadly described above. The constructs comprise a vector, such as a plasmid or viral vector, into which a nucleic acid sequence of the invention has been inserted, in a forward or reverse orientation. In a preferred aspect of this embodiment, the construct further comprises regulatory sequences, including, for example, a promoter operably linked to the sequence. Large numbers of suitable vectors and promoters are known to those of skill in the art, and are commercially available. Appropriate cloning and expression vectors for use with prokaryotic and eukaryotic hosts are also described in Sambrook, et al., (supra).

The present invention also relates to host cells which are genetically engineered with vectors of the invention, and the production of the product of the invention by recombinant techniques. Host cells are genetically engineered (i.e., transduced, transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the expression of the CHL2 nucleic acid sequence. The culture conditions, such as temperature, pH and the like, are those previously used with the host cell selected for expression, and will be apparent to those skilled in the art.

The nucleic acid sequences of the present invention may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence (promoter) to direct mRNA synthesis. Examples of such promoters include: LTR or SV40 promoter, the E. coli lac or trp promoter, the phage lambda PL promoter, and other promoters known to control expression of genes in prokaryotic or eukaryotic cells or their viruses. The expression vector also contains a ribosome binding site for translation initiation, and a transcription terminator. The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sf9; animal cells such as CHO, COS, HEK 293 or Bowes melanoma; adenoviruses; plant cells, etc. The selection of an appropriate host is deemed to be within the scope of those skilled in the art from the teachings herein. The invention is not limited by the host cells employed.

In bacterial systems, a number of expression vectors may be selected depending upon the use intended for the CHL2 product. For example, when large quantities of CHL2 product are needed for the induction of antibodies, vectors which direct high level expression of fusion proteins that are readily purified may be desirable. Such vectors include, but are not limited to, multifunctional E. coli cloning and expression vectors such as Bluescript(R) (Stratagene), in which the CHL2 polypeptide coding sequence may be ligated into the vector in-frame with sequences for the amino-terminal Met and the subsequent 7 residues of beta-galactosidase so that a hybrid protein is produced; pIN vectors (Van Heeke & Schuster J. Biol. Chem. 264:5503-5509, (1989)); pET vectors (Novagen, Madison Wis.); and the like.

In the yeast Saccharomyces cerevisiae a number of vectors containing constitutive or inducible promoters such as alpha factor, alcohol oxidase and PGH may be used. For reviews, see Ausubel et al. (supra) and Grant et al., (Methods in Enzymology 153:516-544, (1987)).

In cases where plant expression vectors are used, the expression of a sequence encoding CHL2 product may be driven by any of a number of promoters. For example, viral promoters such as the 35S and 19S promoters of CaMV (Brisson et al., Nature 310:511-514. (1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J. 3:1671-1680, (1984); Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

CHL2 product may also be expressed in an insect system. In one such system, Autographa californica nuclear polyhedrosis virus (AcNPV) is used as a vector to express foreign genes in Spodoptera frugiperda cells or in Trichoplusia larvae. The CHL2 product coding sequence may be cloned into a nonessential region of the virus, such as the polyhedrin gene, and placed under control of the polyhedrin promoter. Successful insertion of CHL2 coding sequence will render the polyhedrin gene inactive and produce recombinant virus lacking coat protein coat. The recombinant viruses are then used to infect S. frugiperda cells or Trichoplusia larvae in which CHL2 protein is expressed (Smith et al., J. Virol. 46:584, (1983); Engelhard, E. K. et al., Proc. Nat. Acad. Sci. 91:3224-7, (1994)).

In mammalian host cells, a number of viral-based expression systems may be utilized. In cases where an adenovirus is used as an expression vector, a CHL2 product coding sequence may be ligated into an adenovirus transcription/translation complex consisting of the late promoter and tripartite leader sequence. Insertion in a nonessential E1 or E3 region of the viral genome will result in a viable virus capable of expressing CHL2 protein in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells.

Specific initiation signals may also be required for efficient translation of a CHL2 protein coding sequence. These signals include the ATG initiation codon and adjacent sequences. In cases where CHL2 product coding sequence, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., (1994) Results Probl. Cell Difer., 20:125-62, (1994); Bittner et al., Methods in Enzymol 153:516-544, (1987)).

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre-pro” form of the protein may also be important for correct insertion, folding and/or function. Different host cells such as CHO, HeLa, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express CHL2 product may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler M., et al., Cell 11:223-32, (1977)) and adenine phosphoribosyltransferase (Lowy I., et al., Cell 22:817-23, (1980)) genes which can be employed in tk- or aprt-cells, respectively. Also, antimetabolite, antibiotic or herbicide resistance can be used as the basis for selection; for example, dhfr which confers resistance to methotrexate (Wigler M., et al., Proc. Natl. Acad. Sci. 77:3567-70, (1980)); npt, which confers resistance to the aminoglycosides neomycin and G-418 (Colbere-Garapin, F. et al., J Mol. Biol, 150:1-14, (1981)) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad. Sci. 85:8047-51, (1988)). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes, C. A. et. al., Methods Mol. Biol., 55:121-131, (1995)).

Host cells transformed with a nucleotide sequence encoding CHL2 product may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors containing nucleic acid sequences encoding CHL2 product can be designed with signal sequences which direct secretion of CHL2 product through a prokaryotic or eukaryotic cell membrane.

The CHL2 product may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.). The inclusion of a protease-cleavable polypeptide linker sequence between the purification domain and CHL2 protein is useful to facilitate purification. One such expression vector provides for expression of a fusion protein compromising a CHL2 polypeptide fused to a polyhistidine region separated by an enterokinase cleavage site. The histidine residues facilitate purification on IMIAC (immobilized metal ion affinity chromatography, as described in Porath, et al., Protein Expression and Purification, 3:263-281, (1992)) while the enterokinase cleavage site provides a means for isolating CHL2 polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

The CHL2 products can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps.

C. Diagnostic Applications Utilizing Nucleic Acid Sequences

The nucleic acid sequences of the present invention may be used for a variety of diagnostic purposes. The nucleic acid sequences may be used to detect and quantify expression of CHL2 in patient cells, e.g. biopsied tissues, by detecting the presence of mRNA coding for CHL2 product. Alternatively, the assay may be used to detect soluble CHL2 in the serum or blood. This assay typically involves obtaining total mRNA from the tissue or serum and contacting the mRNA with a nucleic acid probe. The probe is a nucleic acid molecule of at least about 20 nucleotides, preferably from about 20 about 30 nucleotides, capable of specifically hybridizing with a sequence included within the sequence of a nucleic acid molecule encoding CHL2 under hybridizing conditions (optionally and more preferably capable of detecting a bridge portion of the nucleic acid molecule), detecting the presence of mRNA hybridized to the probe, and thereby detecting the expression of CHL2. This assay can be used to distinguish between absence, presence, and excess expression of CHL2 product and to monitor levels of CHL2 expression during therapeutic intervention.

Preferably the probe is such that it detects sequences present in CHL2 group I sequences and not present in other chordin-like homologs, such as Group II sequences for example.

The invention also contemplates the use of the nucleic acid sequences for diagnosing diseases resulting from mutated CHL2 sequences. These sequences can be detected by comparing the sequences of the defective (i.e., mutant) CHL2 coding region with that of a normal coding region_of CHL2. Association of the sequence coding for mutant CHL2 product with abnormal CHL2 product activity may be verified. In addition, sequences encoding mutant CHL2 products can be inserted into a suitable vector for expression in a functional assay system (e.g., calorimetric assay, complementation experiments in a CHL2 protein deficient strain of HEK293 cells) as yet another means to verify or identify mutations. Once mutant genes have been identified, one can then screen populations of interest for carriers of the mutant gene.

Individuals carrying mutations in the nucleic acid sequence of the present invention may be detected at the DNA level by a variety of techniques. Nucleic acids used for diagnosis may be obtained from patient cells, including but not limited to cells derived from blood, urine, saliva, placenta, tissue biopsy and autopsy material and amniotic fluid. Genomic DNA may be used directly for detection or may be amplified enzymatically by PCR (Saiki, et al., Nature 324:163-166, (1986)) prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype.

Point mutations can be identified by hybridizing amplified DNA to radiolabeled RNA of the invention or alternatively, radiolabeled antisense DNA sequences of the invention. Sequence changes at specific locations may also be revealed by nuclease protection assays, such RNase and SI protection or the chemical cleavage method (e.g. Cotton, et al. Proc. Natl. Acad. Sci. USA, 85:4397-4401, (1985)), or by differences in melting temperatures. “Molecular beacons” (Kostrikis L. G. et al., Science 279:1228-1229, (1998)), hairpin-shaped, single-stranded synthetic oligonucleotides containing probe sequences which are complementary to the nucleic acid of the present invention, may also be used to detect point mutations or other sequence changes as well as monitor expression levels of the CHL2 product. Such diagnostics would be particularly useful for prenatal testing.

Another method for detecting mutations uses two DNA probes which are designed to hybridize to adjacent regions of a target, with abutting bases, where the region of known or suspected mutation(s) is at or near the abutting bases. The two probes may be joined at the abutting bases, e.g., by a ligation reaction, but only if both probes are correctly base paired in the region of probe junction. The presence or absence of mutations is then detectable by the presence or absence of ligated probe.

Also suitable for detecting mutations in the CHL2 product coding sequence are oligonucleotide array methods based on sequencing by hybridization (SBH), as described, for example, in U.S. Pat. No. 5,547,839. In a typical method, the DNA target analyte is hybridized with an array of oligonucleotides formed on a microchip. The sequence of the target can then be “read” from the pattern of target binding to the array.

E. Therapeutic Applications of Nucleic Acid Sequences

Nucleic acid sequences of the invention may also be used for therapeutic purposes. Turning first to the second aspect of the invention (i.e. inhibition of expression of CHL2), expression of the CHL2 product may be modulated through antisense technology, which controls gene expression through hybridization of complementary nucleic acid sequences, i.e. antisense DNA or RNA, to the control, 5′ or regulatory regions of the gene encoding CHL2 product. For example, the 5′ coding portion of the nucleic acid sequence which codes for the product of the present invention is used to design an antisense oligonucleotide of from about 10 to 40 bases in length. Oligonucleotides derived from the transcription CHL2 site, e.g. between positions −10 and +10 from the CHL2t site, are preferred. An antisense DNA oligonucleotide is designed to be complementary to a region of the nucleic acid sequence involved in transcription (Lee et al., Nucl. Acids, Res., 6:3073, (1979); Cooney et al., Science 241:456, (1988); and Dervan et al., Science 251:1360, (1991)), thereby preventing transcription and the production of the CHL2 products. An antisense RNA oligonucleotide hybridizes to the mRNA in vivo and blocks translation of the mRNA molecule into the CHL2 products (Okano J. Neurochem. 56:560, (1991)). The antisense constructs can be delivered to cells by procedures known in the art such that the antisense RNA or DNA may be expressed in vivo. The antisense may be antisense mRNA or DNA sequence capable of coding such antisense mRNA. The antisense mRNA or the DNA coding thereof can be complementary to the full sequence of nucleic acid sequences encoding the CHL2 protein or to a fragment of such a sequence which is sufficient to inhibit production of a protein product.

The efficiency of the antisense therapy can then be assessed by labeled CHL2 oligonucleotides, according to techniques described in Younes et al. (Younes C. K., Boisgard R. and Tavitian B., 2002, Labelled Oligonucleotides as Radiopharmaceuticals: Pitfalls, Problems and Perspectives. Current Pharmaceutical Design, 8: 1451-1466). The labeled antisense oligonucleotids themselves may also be used for therapy, i.e. gene radiotherapy, as described in Younes et al.

Antisense oligonucleotides of the invention may also be used in order to alter the alternative splicing pattern of CHL2, according to the techniques described in Sazani et al. (Modulation of alternative splicing by antisense oligonucleotides. Sazani, P. and Kole R., 2003, Progress in Molecular and Subcellular biology, 31: 217-239). According to this approach, CHL2 antisense oligonucleotides of the invention may be used in order to correct aberrant splicing in diseases that are caused by such an aberrant splicing of CHL2. Such antisense oligonucleotides may also be used in cases where the splicing is correct, yet shifting of the ratio between the splice variants is desirable.

In such cases, CHL2 antisense oligonucleotides of approximately 20 bases are designed to correspond to specific splice branch points of the CHL2 gene. Alternatively, the oligonucleotides may be targeted to the aberrant splice site, thereby, presumably and without being limited by theory, blocking use of the aberrant splice site and shifting splicing back to the normal or desirable pattern. The antisense oligonucleotides may also be targeted to other sites in the gene, as can be determined by routine experimentation, such as splice donor or acceptor sites, or any other sites in the intron or the exon that might affect splicing or influence the splicing machinery.

Modulation of alternative splicing by antisense oligonucleotides may be done in vivo, by administering the oligonucleotides to a patient in need, or ex vivo, whereby the treated cells are then returned to the patient's body.

RNA interference is a two step process. During the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews: Tuschl, ChemBiochem. 2:239-245 (2001); Cullen Nat. Immunol. 3:597-599 (2002); and Brantl, Biochem. Biophys. Act. 1575:15-25 (2002); all of which are hereby incorporated by reference as if fully set forth herein.

Synthesis of RNAi molecules suitable for use with the present invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as templates for siRNA synthesis. Preferred sequences are those including low G/C content as these have proven to be more effective in mediating gene silencing as compared to those with G/C content higher than 55%. Several target sites are preferably selected along the length of the target gene for evaluation. Target sites are selected from the unique nucleotide sequences of each of the polynucleotides of the present invention, such that each polynucleotide is specifically down regulated.

Turning now to an important aspect of the invention, i.e. increased expression of CHL2, expression of CHL2 product may be increased by providing sequences encoding said product under the control of suitable control elements which determine the timing and location of its expression in the desired host.

The nucleic acid sequences of the invention may be employed in combination with a suitable pharmaceutical carrier. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The formulation should suit the mode of administration.

The products of the invention as well as any activators and deactivators compounds (see below) which are polypeptides, may also be employed in accordance with the present invention by expression of such polypeptides in vivo, which is often referred to as “gene therapy”. Cells from a patient may be engineered with a nucleic acid sequence (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a polypeptide of the present invention.

Similarly, cells may be engineered in vivo for expression of a polypeptide in vivo by procedures known in the art. As known in the art, a producer cell for producing a retroviral particle containing RNA encoding the polypeptide of the present invention may be administered to a patient for engineering cells in vivo and expression of the polypeptide in vivo. These and other methods for administering a product of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses from which the retroviral plasmid vectors mentioned above may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PESO, PA317, psi-2, psi-AM, PA12, T19-14X, VT-19-17-H2, psi-CRE, psi-CRIP, GP+E-86, GP+envAm12, and DAN cell lines as described in Miller (Human Gene Therapy, Vol. 1, pg. 5-14, (1990)). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

The genes introduced into cells may be placed under the control of inducible promoters, such as the radiation-inducible Egr-1 promoter, (Maceri, H. J., et al., Cancer Res., 56(19):4311 (1996)), to stimulate CHL2 production or antisense inhibition in response to radiation, eg., radiation therapy for treating tumors.

### EXAMPLE II

### The CHL2 Product

The substantially purified CHL2 product of the invention has been defined above as the product coded, from the nucleic acid sequence of the invention. Preferably the amino acid sequence is an amino acid sequence having at least about 70% but preferably at least about 80%, preferably at least 90% or 95% or 98% identity to the sequence identified as any one of SEQ ID NO: 11 to SEQ ID NO: 15 (Group I sequences). Optionally, the amino acid sequence is an amino acid sequence having at least about 70% but preferably at least about 80%, preferably at least 90% or 95% or 98% identity to the sequence identified as any one of SEQ ID NO: 16 to SEQ ID NO: 20 or SEQ ID NO: 85 to SEQ ID NO: 95 (Group II sequences). The protein or polypeptide may be in mature and/or modified form, also as defined above. Also contemplated are protein fragments having at least 10 contiguous amino acid residues, preferably at least 10-20 residues, derived from the CHL2 product.

The sequence variations are preferably those that are considered conserved substitutions, as defined above. Thus, for example, a protein with a sequence having at least about 70% but preferably at least about 80%, more preferably about 90% and most preferably at least about 95% sequence identity with the protein identified as any one of SEQ ID NO: 11 to SEQ ID NO: 1 (Group I sequences), preferably by utilizing conserved substitutions as defined above is also part of the invention. Optionally as noted above, a protein with a sequence having at least about 70% but preferably at least about 80%, more preferably about 90% and most preferably at least about 95% sequence identity with the protein identified as any one of SEQ ID NO: 16 to SEQ ID NO: 20 or SEQ ID NO: 85 to SEQ ID NO: 95 (Group II sequences), preferably by utilizing conserved substitutions as defined above is also part of the invention.

In a more specific embodiment, the protein has or contains the sequence identified as any one of SEQ ID NO: 11 to SEQ ID NO: 15 (Group I sequences). Optionally, the protein has or contains the sequence identified as any one of SEQ ID NO: 16 to SEQ ID NO: 20 or SEQ ID NO: 85 to SEQ ID NO: 95 (Group II sequences).

In any case, the resultant protein product may be (i) one in which one or more of the amino acid residues in a sequence listed above are substituted with a conserved or non-conserved amino acid residue (preferably a conserved amino acid residue), or (ii) one in which one or more of the amino acid residues includes a substituent group, or (iii) one in which the CHL2 product is fused with another compound, such as a compound to increase the half-life of the protein (for example, polyethylene glycol (PEG), or a moiety which serves as targeting means to direct the protein to its target tissue or target cell population (such as an antibody), or (iv) one in which additional amino acids are fused to the CHL2 product. Such fragments, variants and derivatives are deemed to be within the scope of those skilled in the art from the teachings herein.

A. Preparation of CHL2 Product

Recombinant methods for producing and isolating the CHL2 product, and fragments of the protein are described above.

In addition to recombinant production, fragments and portions of CHL2 product may be produced by direct peptide synthesis using solid-phase techniques (cf Stewart et al., (1969) Solid-Phase Peptide Synthesis, WH Freeman Co, San Francisco; Merrifield J., J. Am. Chem. Soc., 85:2149-2154, (1963)). In vitro peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. Fragments of CHL2 product may be chemically synthesized separately and combined using chemical methods to produce the full length molecule.

B. Therapeutic Uses and Compositions Utilizing the CHL2 Product

The CHL2 product of the invention is generally useful in treating diseases and disorders which are characterized by a lower than normal level of CHL2 expression, and or diseases which can be cured, ameliorated or prevented by raising the level of the CHL2 product, even if the level is normal.

Typically the CHL2 products or fragments may be administered by any of a number of routes and methods designed to provide a consistent and predictable concentration of compound at the target organ or tissue. The product-containing compositions may be administered alone or in combination with other agents, such as stabilizing compounds, and/or in combination with other pharmaceutical agents such as drugs or hormones.

CHL2 product-containing compositions may be administered by a number of routes including, but not limited to oral, intravenous, intramuscular, transdermal, subcutaneous, topical, sublingual, or rectal means as well as by nasal application. CHL2 product-containing compositions may also be administered via liposomes. Such administration routes and appropriate formulations are generally known to those of skill in the art.

The product can be given via intravenous or intraperitoneal injection. Similarly, the product may be injected to other localized regions of the body. The product may also be administered via nasal insufflation. Enteral administration is also possible. For such administration, the product should be formulated into an appropriate capsule or elixir for oral administration, or into a suppository for rectal administration.

The foregoing exemplary administration modes will likely require that the product be formulated into an appropriate carrier, including ointments, gels, suppositories. Appropriate formulations are well known to persons skilled in the art.

Dosage of the product will vary, depending upon the potency and therapeutic index of the particular polypeptide selected.

A therapeutic composition for use in the treatment method can include the product in a sterile injectable solution, the polypeptide in an oral delivery vehicle, the product in an aerosol suitable for nasal administration, or the product in a nebulized form, all prepared according to well known methods. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The product of the invention may also be used to modulate endothelial differentiation and proliferation as well as to modulate apoptosis either ex vivo or in vitro, for example, in cell cultures.

### EXAMPLE III

### Screening Methods for Activators and Deactivators (Inhibitors)

The present invention also includes an assay for identifying molecules, such as synthetic drugs (small organic molecules), antibodies, peptides, or other molecules, which have a modulating effect on the activity of the CHL2 product, e.g. activators or deactivators of the CHL2 product of the present invention. Such an assay comprises the steps of providing an CHL2 product encoded by the nucleic acid sequences of the present invention and determining its physiological activity on the target in the presence and absence of one or more candidate molecules to determine the candidate molecules. Those molecules which have a modulating effect on the activity of the CHL2 product are selected as likely candidates for activators and deactivators.

The CHL2 product, its catalytic or immunogenic fragments or oligopeptides thereof, can be used for screening therapeutic compounds in any -of a variety of drug screening techniques. The fragment employed in such a test may be free in solution, affixed to a solid support, borne on a cell membrane or located intracellularly. The formation of binding complexes, between CHL2 product and the agent being tested, may be measured. Alternatively, the activator or deactivator may work by serving as agonist or antagonist, respectively, of the CHL2 receptor and their effect may be determined in connection with the receptor.

Another technique for drug screening which may be used provides for high throughput screening of compounds having suitable binding affinity to the CHL2 product is described in detail by Geysen in PCT Application WO 84/03564, published on Sep. 13, 1984. In summary, large numbers of different small peptide test compounds are synthesized on a solid substrate, such as plastic pins or some other surface. The peptide test compounds are reacted with the full CHL2 product or with fragments of CHL2 product and washed. Bound CHL2 product is then detected by methods well known in the art. Substantially purified CHL2 product can also be coated directly onto plates for use in the aforementioned drug screening techniques. Alternatively, non-neutralizing antibodies can be used to capture the peptide and immobilize it on a solid support.

Antibodies to the CHL2 product, as described in Example IV below, may also be used in screening assays according to methods well known in the art. For example, a “sandwich” assay may be performed, in which an anti-CHL2 antibody is affixed to a solid surface such as a microtiter plate and the CHL2 product is added. Such an assay can be used to capture compounds which bind to the CHL2 product. Alternatively, such an assay may be used to measure the ability of compounds to influence with the binding of CHL2 product to the CHL2 receptor and then select those compounds which effect the binding.

### EXAMPLE IV

### Anti-CHL2 Antibodies

A. Synthesis

In still another aspect of the invention, the purified CHL2 product is used to produce anti-CHL2 antibodies which have diagnostic and therapeutic uses related to the activity, distribution, and expression of the CHL2 product, in particular therapeutic applications mentioned in connection with the pharmaceutical composition aspect of the invention. Optionally and preferably, these antibodies specifically bind to an epitope on any Group I sequence, or alternatively (or additionally) to an epitope on any Group II sequence. As noted above, optionally and preferably an epitope comprises a unique bridge portion of an amino acid sequence for a Group I sequence. Preferably, for antibodies specifically binding to an epitope on any Group I sequence, binding is at least two fold higher than binding to an epitope on a Group II sequence and/or to chordin like homolog as described with regard to accession numbers AX175130 or AF209928 (nucleotide sequences; or the corresponding amino acid sequences to these nucleotide sequences).

Antibodies to CHL2 product may be generated by methods well known in the art.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample; for example, more preferably the antibodies bind specifically to one or more Group I sequences but do not bind to one or more Group II sequences (and/or bind at a much lower level, preferably being less than about half the level of binding to one or more Group I sequences). Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

B. Diagnostic Applications of Antibodies

Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of a number of well recognized immunological binding assays (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. For a review of the general immunoassays, see also, Methods in Cell Biology: Antibodies in Cell Biology, volume 37 (Asai, ed. 1993); Basic and Clinical Immunology (Stites & Terr, eds., 7th ed. 1991).

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a substrate as described above. The sample is preferably a biological fluid sample taken from a subject. Examples of biological fluid samples include blood, serum, urine, prostatic fluid, seminal fluid, semen, seminal plasma and prostate tissue (e.g., epithelial tissue, including extracts thereof) as well as amniotic fluid. In a preferred embodiment, the biological fluid comprises seminal plasma. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay techniques are well-known in the art, and a general overview of the applicable technology can be found in the references described above and incorporated by reference.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. As noted above, the test amount of marker need not be measured in absolute units, as long as the unit of measurement can be compared to a control amount.

C. Therapeutic Uses of Antibodies

In addition to their diagnostic use the antibodies may have a therapeutic utility in blocking or decreasing the activity of the CHL2 product in pathological conditions where beneficial effect can be achieved by such a decrease. Preferably, the antibody blocks or decreases activity of a Group I polypeptide, but optionally the antibody blocks or decreases activity of a Group II polypeptide.

The antibody employed is preferably a humanized monoclonal antibody, or a human mAb produced by known globulin-gene library methods. The antibody is administered typically as a sterile solution by IV injection, although other parenteral routes may be suitable. Typically, the antibody is administered in an amount between about 1-15 mg/kg body weight of the subject. Treatment is continued, e.g., with dosing every 1-7 days, until a therapeutic improvement is seen.

Although the invention has been described with reference to specific methods and embodiments, it is appreciated that various modifications and changes may be made without departing from the invention.

### EXAMPLE V

### Experimental Procedures

RT-PCR

Total RNA was extracted from cells using the Tri-Reagent (Molecular Research Center Inc., Cincinnati, Ohio). Total RNA samples from human tissues were purchased from BioChain Inc. (Hayward, Calif.) and from Ambion, Inc. (Austin, Tex.). Reverse transcription (RT) on 1 μg of total RNA was carried out as previously described (David et al., 2002). RT-PCR was performed in 25 μl reactions using 1 μl of RT reaction, in the presence of 2 mM dNTPs, 25 pmol of primers, and 1.25 units of Takara Ex Taq™ Hot Start Version (Takara Shuzo Co., LTD, Japan) in the reaction buffer supplied by the manufacturer. RT-PCR products were analyzed on agarose gels, and their identity was verified by DNA sequencing.

RACE Analysis of 5′ End

Poly (A) RNA was isolated from total RNA using (dT)25 Dynabeads (Dynal, Oslo, Norway). Rapid amplification of cDNA ends (RACE) analysis was performed on poly (A) RNA from human placenta, testis and brain tissues using the Marathon cDNA Amplification Kit (Clontech, Palo Alto, Calif.). Adaptor-ligated, double-stranded cDNA libraries were prepared essentially as suggested by the manufacturer. Superscript II Reverse Transcriptase (Invitrogen) was used for the first strand synthesis. First round PCR was performed on these libraries using the Expand Long Template PCR System (Roche Molecular Biochemicals). All PCRs were carried out in the presence of 1.5 μg of anti-Taq monoclonal antibody (Clontech, Palo Alto, Calif.). A nested PCR approach was used to isolate 5′-RACE products, and the presence of these transcripts was subsequently verified by RT-PCR.

Northern Blot Analysis

Multiple Tissue Northern blots from Clontech (Palo Alto, Calif.) were used: Human Muscle MTN™ and Human Fetal MTN™ blots containing poly (A) RNA samples from adult and fetal human tissues, respectively. The blots were hybridized essentially as described in David et al (2002). The probe used for the hybridization of the adult human tissue blot was a DNA fragment containing nucleotides 1041-1669 from the hCHL2 ORF (variant II, SEQ ID NO:6), obtained by RT-PCR using the forward primer p9, from the beginning of exon 8, and the reverse primer p8 (SEQ ID NO: 28). The probe used for the hybridization of the fetal human tissue blot was a DNA fragment containing nucleotides 271-1669 from the hCHL2 ORF (variant II, SEQ ID NO:6), obtained by RT-PCR using the forward primer p1 (SEQ ID NO:21) and the reverse primer p8 (SEQ ID NO: 28).

Preparation of Antibodies

An amplified DNA fragment containing the coding region of hCHL2 (Variant II, SEQ ID NO:6) was cloned into the GST-fusion vector pGEX-6P (Amersham Biosciences AB, Uppsala, Sweden). E. coli DH5α cells were transformed with this construct, and the encoded recombinant GST-hCHL2 protein was expressed, purified and cleaved following the manufacturer's instructions. Polyclonal antibodies were raised by immunizing rabbits with the purified GST-hCHL2 fused protein.

Immunohistochemistry

Different human tissue samples were fixed in 10% buffered formalin, and 3 μm sections were prepared using a R. Gung microtome and mounted on OptiPlus™ slides (BioGenex, San Ramon, Calif.). Deparaffinization was performed with xylene for 10 min, and the sections were rehydrated by rinsing three times with 100% ethanol and once with 95% ethanol. Slides were washed in double distilled H2O and then incubated with 3% H2O2 for 5 min. Subsequently, the slides were washed again in double distilled H2O and twice in OptimaxTM Wash Buffer (BioGenex, San Ramon, Calif.). Immunohistochemical staining was performed using HistostainTM Plus Bulk Kit (Zymed Laboratories Inc., San Francisco, Calif.). The anti-hCHL2 polyclonal antibody, raised in the course of this study, was used at 1:750 dilution. Counterstaining with haematoxylin was employed.

Mammalian Expression Constructs and Cell Transfections

The complete coding sequences of hCHL2 (Variant II, SEQ ID NO:6) was cloned into the pCDNA3 and the pcDNA4/myc-His mammalian expression vectors (Invitrogen, Carlsbad, Calif.). The latter vector enables the synthesis of proteins fused to the Myc and His epitope tags at the C-terminus. COS-7 cells, at about 70% confluency, were transfected with the expression constructs or empty vectors using the FuGENETM 6 transfection reagent (Boehringer Mannheim), according to the manufacturer's instructions. On the day following transfection, the growth medium was replaced with 0.1% FCS containing fresh media. Culture media were collected after 48 and 72 hrs, and the cells were washed with PBS, harvested and lysed in 150 mM NaCl, 50 mM Tris pH 8.0, 1% NP40, 0.5% DOC, 0.1% SDS and 10% glycerol.

Immunoblot Analysis

Cell lysates or culture media were fractionated on NuPAGETM 4-12% gradient Bis-Tris gel (Invitrogen), transferred onto Immun-BlotTM PVDF membrane (Bio-Rad, Hercules, Calif.) and subjected to immunodetection using the rabbit immunized anti-hCHL2 sera described above or the following commercial antibodies: anti-human BMP2/4 rabbit polyclonal antibody, anti-human BMP-6 goat polyclonal antibody from Santa Cruz Biotechnology, Inc. (Santa Cruz, Calif.), anti-human Activin A goat polyclonal antibody from R&D Systems. Peroxidase-conjugated Donkey Anti-Rabbit or anti-Goat IgGs (Jackson ImmunoResearch Laboratories, West Grove, Pa.) were used as secondary antibodies. Proteins were visualized with the SuperSignal West Pico or West Femto ECL systems (Pierce, Rockford, Ill.).

Protein binding assays with His-tagged hCHL2 protein Culture media of COS7 cells, transfected with hCHL2 cloned in pcDNA4/His, or empty vector (see above), were collected every 24 hrs, beginning at 48 hrs after transfection for a total of 96 hrs, and concentrated 10-fold by ultrafiltration through a YM Millipore membrane under nitrogen pressure (Millipore, Bedford, Mass.) before binding of secreted His-tagged hCHL2 to BD TALONTM Metal Affinity Resin (Clontech, Palo Alto, Calif.). The resin was added to the concentrated medium, rotated for 30 min at room temperature, and then washed twice with loading buffer (50 mM NaH₂PO₄, pH 8, 300 mM NaCl, 5 mM imidazole) and resuspended in 200 μl of binding buffer (1 mM CaCl₂, 3 mM MgCl₂, 0.2% NP-40, 1 mg/ml BSA in PBS). The His-hCHL2, immobilized on the resin, was incubated with 200 ng of recombinant human BMP-2, -4, -6 or Activin A (all purchased from R&D Systems), while rotating for 1 hr at room temperature, and then washed once with binding buffer and twice with binding buffer supplemented with 0.2% Tween. Subsequently, the resin was resuspended in PAGE sample buffer and incubated for 10 min at 70° C. The supernatant was then subjected to immunoblot analysis with the respective antibodies.

Cell Cultures and Reagents

The African green monkey kidney fibroblast cell line COS-7 (ATCC CRL-1651, Manassas, Va.) was propagated in Dulbecco's modified Eagle's medium (DMEM, Biological Industries, Beit Haemek, Israel) containing 10% fetal calf serum (FCS, Biological Industries). The human osteosarcoma fibroblast cell line MG-63 (ATCC CRL-1427) was propagated in DMEM containing 10% FCS, and the human osteosarcoma epithelial cell line SaOS-2 (ATCC HTB-85) was maintained in McCoy's SA medium (Biological Industries, Beit Haemek, Israel) supplemented with 10% FCS. All cell lines were incubated at 37° C. under 5% CO2. Induction of osteoblasts maturation was carried out by incubating cells in medium without serum, containing 100 nM human PTH (parathyroid hormone) 1-34 (Calbiochem, San Diego, Calif.) for up to 72 hrs. The murine promyoblast cell line, C2C12 (ATCC CRL-1772) was maintained in DMEM supplemented with 15% FCS. Differentiation of C2C12 cells to myotubes was carried out in medium without serum, containing 10 μg/ml insulin, while differentiation of these cells to osteoblasts was carried out in media with 2% serum and 300 ng/ml BMP-2 (R&D Systems, Minneapolis, Minn.). Recombinant human BMP-2, BMP-4, BMP-6, and Activin A were purchased from R&D Systems.

### EXAMPLE VI

### Expression of the hCHL2 Gene in Adult Human Tissues

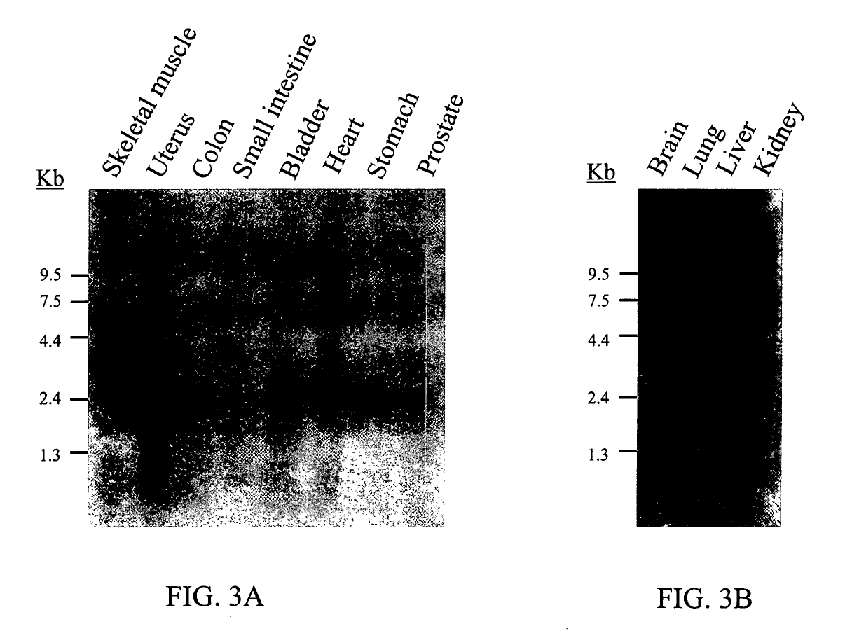

Northern blot analysis, using probe amplified by PCR using primers p9 and p8 (SEQ ID NO: 28, 29), or primers p1 and p8 (SEQ ID NOs: 21, 28) (FIGS. 3A and 3B, respectively) revealed a major 2.3 Kb hCHL2 transcript, which was highly expressed in uterus and was also detected in a variety of human tissues including colon, bladder, heart, stomach, prostate, ovary and testis (FIG. 3). The probe amplified by PCR using primers p9 and p8 (SEQ ID NO: 28, 29), used for hybridization for Northern blot containing poly(A) RNA of human adult tissues (FIG. 3A), spans the sequences between exons 8-11 of CHL2, and it can detect any CHL2 variant accordingly. The probe amplified by PCR using primers p1 and p8 (SEQ ID NO: 28, 21), used for hybridization for Northern blot containing poly(A) RNA of human fetal tissues (FIG. 3B), spans the sequences between exons 1-11 of CHL2, and it can detect any CHL2 variant accordingly. This pattern of expression is further supported by the presence of eleven human ESTs in dbEST (an EST database)—four derived from uterus, two from colon, two from prostate and one from testis. The two remaining ESTs found in the database were from liver and kidney, two tissues that were not present on the northern blots that were analyzed. The size of this 2.3 Kb transcript is larger than the 1.7 Kb of the RACE product, or any other cDNA deposited in the public database. This may be due to additional untranslated sequences in the 5′ and/or 3′ UTRs.

### EXAMPLE VII

### hCHL2 Protein Expression and Secretion

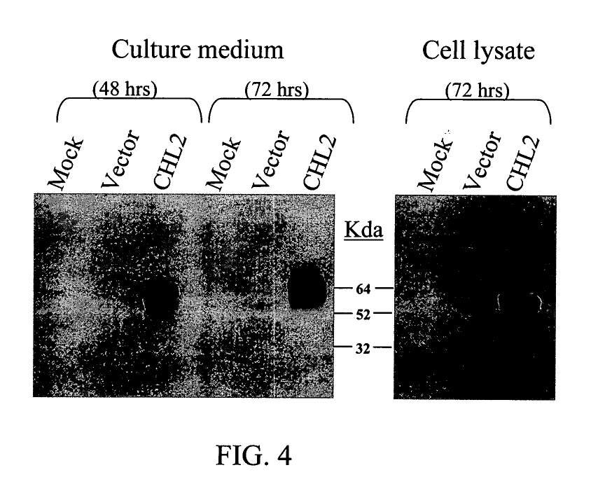

Polyclonal Anti-hCHL2 rabbit serum was generated against a bacterial recombinant hCHL2 protein (variant II, SEQ ID NO:16). The specificity of the immunized sera was analyzed following ectopic expression of hCHL2. For this purpose, the full coding sequence (SEQ ID NO:6) was cloned in the mammalian expression vector pcDNA3 (Invitrogen, Carlsbad, Calif.), and transfected into COS-7 cells (ATCC CRL-165 1, Manassas, Va.). The culture medium was collected after 48 and 72 hrs, at which point the cells were harvested and lysed. Immunoblot analysis, using the anti-hCHL2 rabbit polyclonal antibodies, revealed predominant expression of a protein of about 60 kDa only in the pcDNA3-hCHL2 transfected cells (FIG. 4). The observed size of this protein coincides with the predicted molecular weight of the mature hCHL2 protein, as predicted from its amino acid sequence (55 Kda_; SEQ ID NO: 16). Secretion of the hCHL2 protein is clearly evident from its abundant presence in the growth medium of the transfected cell cultures (FIG. 4).

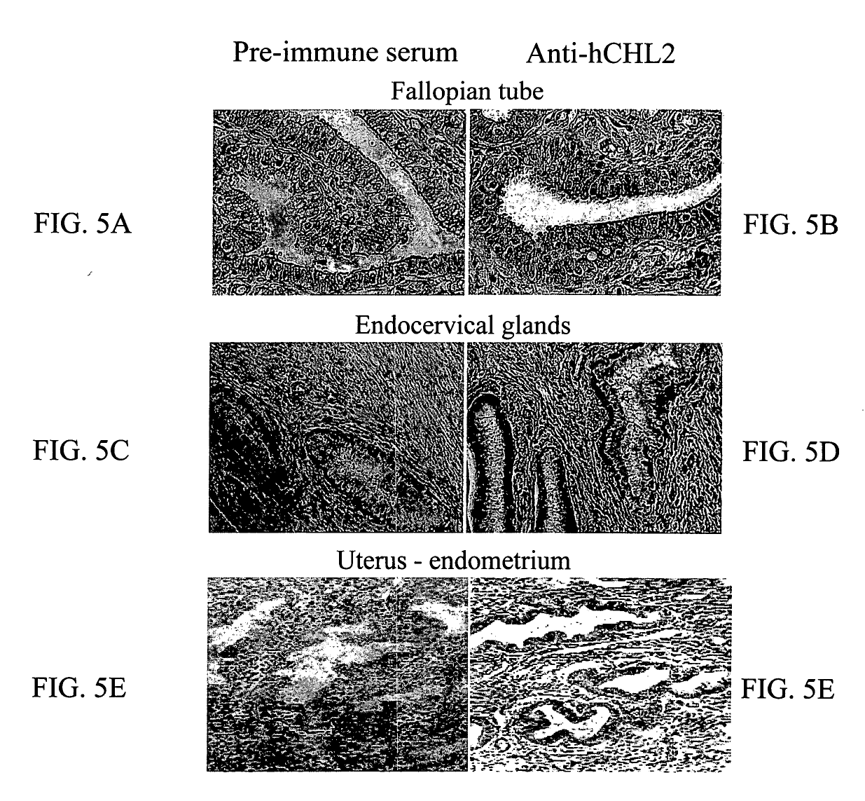

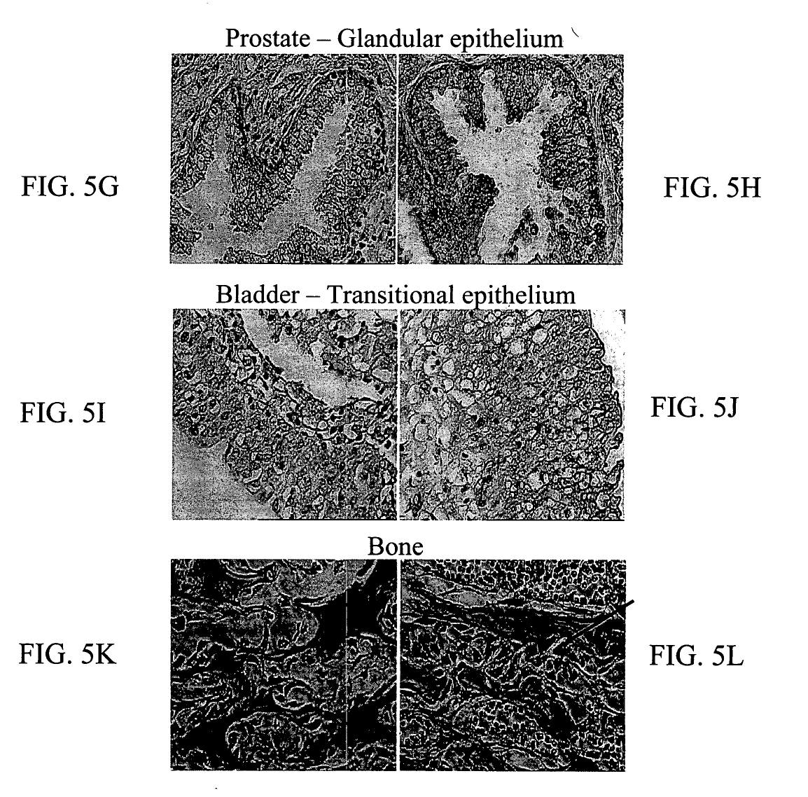

Immunohistochemical analysis of several human tissues using anti-hCHL2 rabbit serum (FIG. 5), indicated expression of this protein in epithelial cells of uterus, fallopian tubes, endocervical glands and bladder. In addition, expression was also evident in bone osteoblasts.

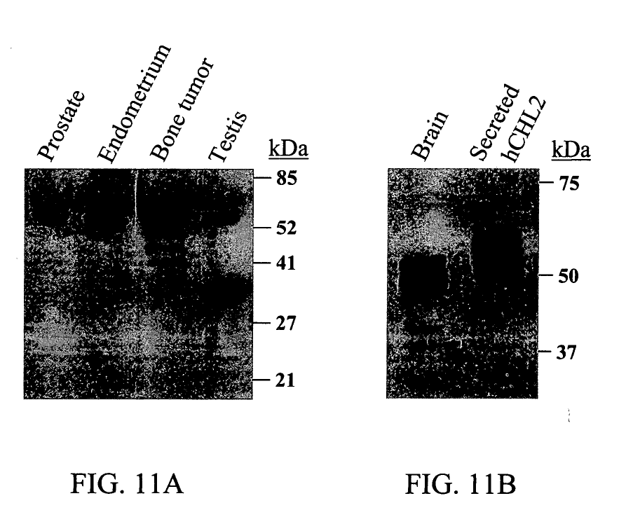

The anti-hCHL2 rabbit serum was also used to analyze the expression of hCHL2 in human tissue by Western blot (FIG. 11). Tissue extracts (60 μg) were fractionated on 10% SDS-PAGE gels and subjected to immunodetection. hCHL2 that was ectopically expressed and secreted from COS-7 cells served as a reference and positive control (FIG. 11B, secreted hCHL2). The hCHL2 protein product is expressed in various tissues, but a particularly high level of expression can be seen in bone tumor cells and in testis. The different protein bands seen in the various tissues might reflect alternative splicing isoforms of hCHL2.

### EXAMPLE VIII

### Direct Interaction of hCHL2 with Activin A

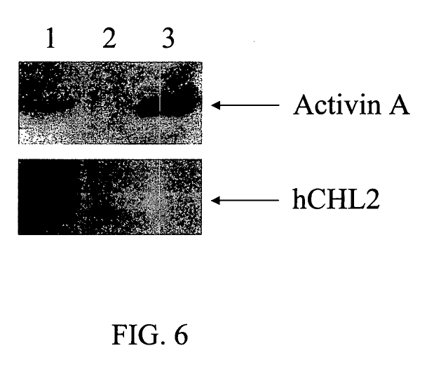

Direct interaction of Chordin and CHL proteins with BMPs and other TGFβ superfamily members, such as Activin A, has been previously demonstrated. hCHL2 interaction with BMP family members was analyzed in an in vitro binding assay. COS-7 cells were transfected with a pcDNA4 (Invitrogen, Carlsbad, Calif.) vector expressing His-tagged hCHL2 (coding region corresponding to variant II, SEQ ID NO:6), and the culture media were collected for 72 hrs. The secreted His-tagged hCHL2 found in the culture media was bound to a metal affinity resin, and incubated with either BMP-2, 4, 6 or Activin A. Immunoblot analysis with the respective antibodies showed binding of Activin A to the immobilized His-hCHL2 (FIG. 6). No interaction was detected with any of the other proteins tested (BMP-2, 4, or 6). These results prove direct interaction of hCHL2 with human Activin A.

### EXAMPLE IX

### Complex Alternative Splice Variants Encode Distinct hCHL2 Isoforms

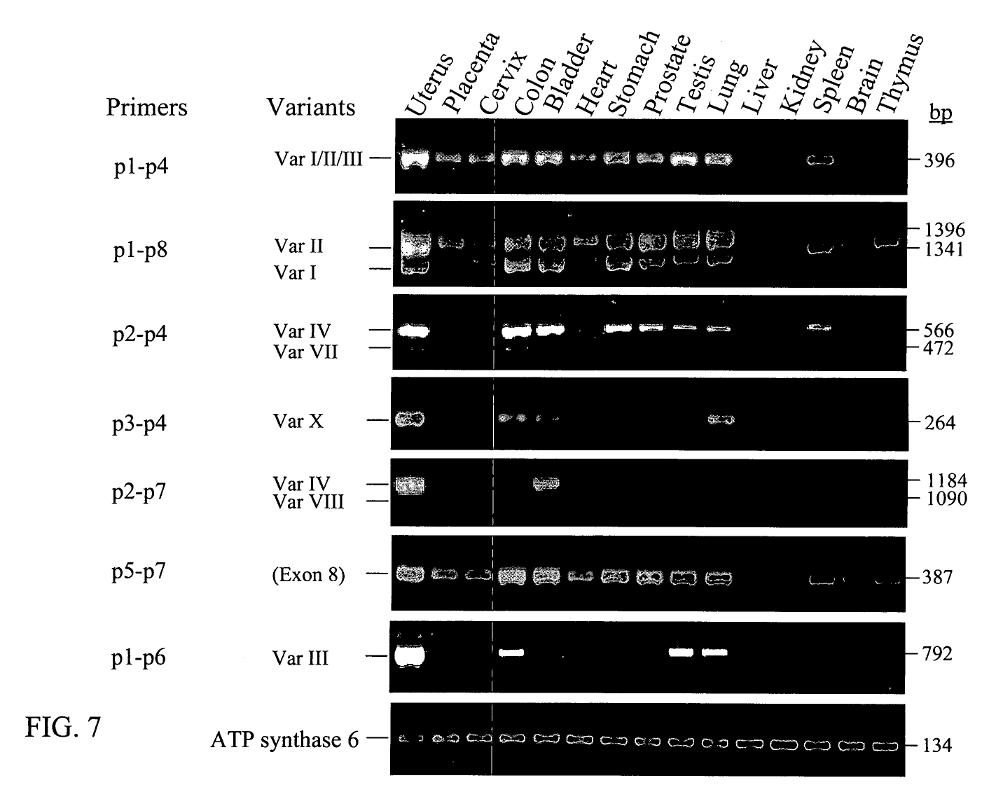

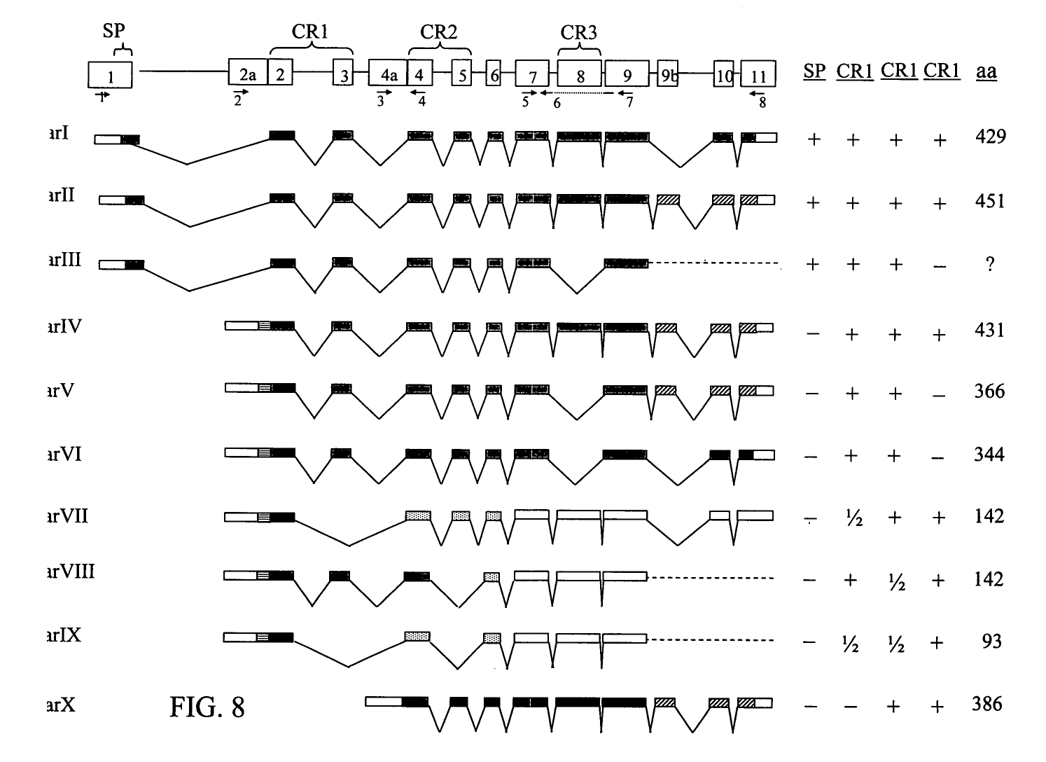

In the course of the RACE analysis of human brain, placenta and testis poly(A) RNA, several alternatively spliced transcripts were identified. Further characterization by RT-PCR in a variety of human tissues (FIG. 7) revealed a complex pattern of alternative splicing resulting in a number of distinct isoforms of the hCHL2 protein. The diagram in FIG. 8 summarizes the various splice variants and the size of the proteins encoded by them, as well as their content of CR repeats and signal peptide.

Three alternative first exons were found. RACE products from placenta, brain and testis indicated the existence of exon 1, which contains a signal peptide immediately downstream of the first in-frame AUG, and therefore encodes for a secreted isoform. RT-PCR analysis with primers 1 and 4 (from exons 1 and 4 respectively—the specific primers used are indicated for each transcript in the figure) indicates that the transcripts initiating in exon 1 (i.e. Vars I, II and III corresponding to SEQ ID NO:1, 6, and 2) are abundant and detected in most of the tissues analyzed (FIG. 7). Different levels of expression were evident from this analysis, with a particularly high level in uterus, in agreement with the results of the northern blot analysis (FIG. 3). These transcripts were not detected in liver and kidney.

RACE analysis and further validation by RT-PCR indicated the existence of two additional alternative first exons: Exon 2a, located within intron 1, adjacent and upstream to exon 2, and Exon 4a, located within intron 3, upstream to exon 4 (FIG. 8). Each of these exons contains an in-frame AUG, providing 8 unique amino acids in exon 2a, and 31 amino acids in exon 4a (just upstream of CR1 or CR2, respectively which do not appear in other CHLs). The protein isoforms translated from these exons have distinct N-termini that lack a signal peptide, and are therefore putative intracellular forms of the hCHL2 protein (encoded by variants IV through IX in FIG. 8, SEQ ID NOs: 3, 4, 5, 7, 8, 9).

RT-PCR with primers 1 and 8, from exons 1 and 11 respectively, revealed the existence of two predominant splice variants that initiate in exon 1 (FIG. 7): in the shorter variant (variant I in FIG. 8 SEQ ID No: 1) exon 9 is joined to exon 10. The longer variant (variant II SEQ ID No: 6) includes an additional exon of 55 nucleotides (exon 9b), between exon 9 and 10. RACE and subsequent RT-PCR analysis indicated the inclusion or exclusion of exon 9b also in variants that initiate within exon 2a (variants IV through VI in FIG. 8, SEQ ID NOs: 7, 8, 9). Inclusion of exon 9b results in a shift to an alternative reading frame of the last two exons, 10 and 11. As a result, the putative isoforms encoded by the variants containing exon 9b have a different C-terminus, as in FIG. 8.

Two additional alternative exons were identified. Exclusion of exon 8 was detected by RACE and RT-PCR. The variants that skip exon 8 are probably rare compared to those containing exon 8, as they are barely detected in some tissues, even after RT-PCR with primers 5 and 7, from exons 7 and 9 respectively (not shown). In order to analyze the expression of this variant, a reverse primer that spans the junction between exons 7 and 9 (primer 6) was designed and used with a forward primer from exon 1 or exon 2a. The results (FIG. 7) indicate that variants that exclude exon 8 are generated from both first exons (variants III, V and VI in FIG. 8, SEQ ID NOs: 2, 8 and 9). Such variants would encode for hCHL2 isoforms that lack CR3, but continue on the same reading frame.

Exclusion of exon 3 was detected by RT-PCR using primers 2 and 4 from exon 2a and exon 4 respectively (FIG. 7; Variant VII in FIG. 8, SEQ ID No: 3). This variant also appears to be very rare, and was detected more clearly with a primer spanning the junction between exon 2 and 4 (data not shown). The putative protein isoform encoded by this variant contains only the first half of CR1. Similarly, Variant VIII (SEQ ID NO: 4), which excludes exon 5, was detected using primers 2 and 7 from exon 2a and exon 9 (FIG. 7 and FIG. 8). This variant codes for a protein isoform containing CR1, and the first half of CR2. Both variants shift to a different reading frame, ending in a stop codon within exon 6.

Variant IX (SEQ ID NO: 5), which initiates in exon 4a, was detected by RACE and verified by RT-PCR. This transcript contains the alternative exons 8 and 9b, thus encoding a putative isoform that lacks a signal peptide and contains CR2 and CR3.

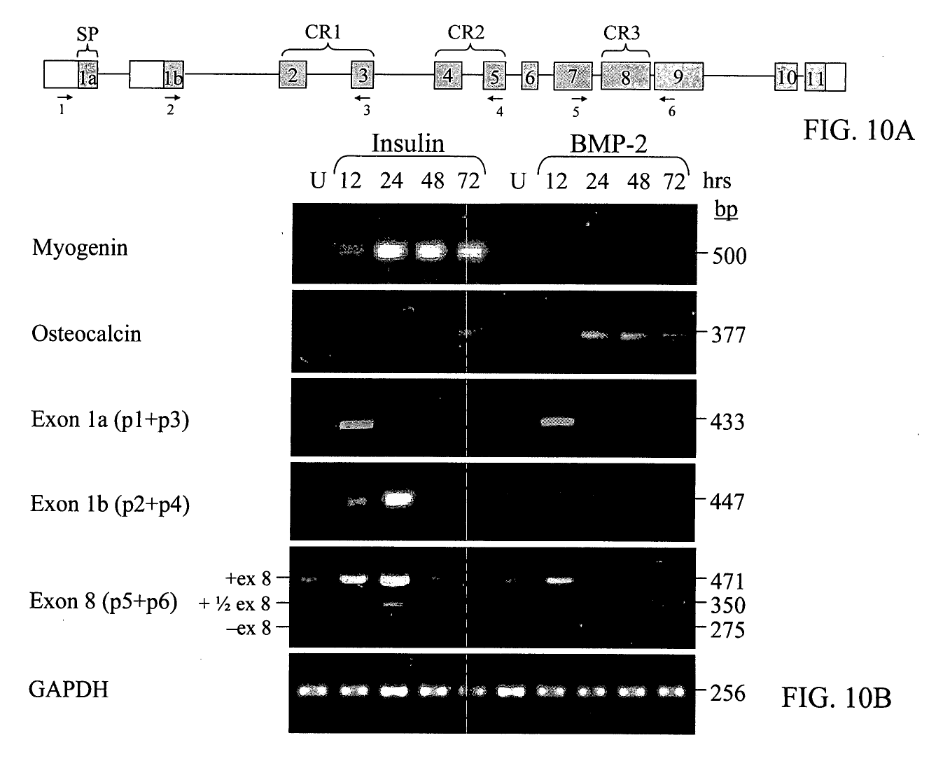

Alternative splicing of the mouse ortholog, mCHL2, is suggested by the various cDNAs and ESTs deposited in GenBank. In addition to murine exon 1a, which is similar to human exon 1, there is an alternative first exon, exon 1b, within intron 1 of mCHL2 (see diagram in FIG. 10A). Exon 1a contains the translation start codon and codes for a cleavable N-terminal signal peptide, while exon 1b contains an AUG codon as translation initiator, but does not encode for a signal peptide. Thus, in analogy to human hCHL2, the murine ortholog also appears to encode for secreted and non-secreted protein isoforms. However, the N-termini of the non-secreted forms differ in the human and mouse genes, as there is no evidence of exon 1b in hCHL2, or exons 2a and 4a in mCHL2.

### EXAMPLE X

### Differential Expression of CHL2 Alternatively Spliced Variants during Differentiation of Osteoblasts and Myoblasts

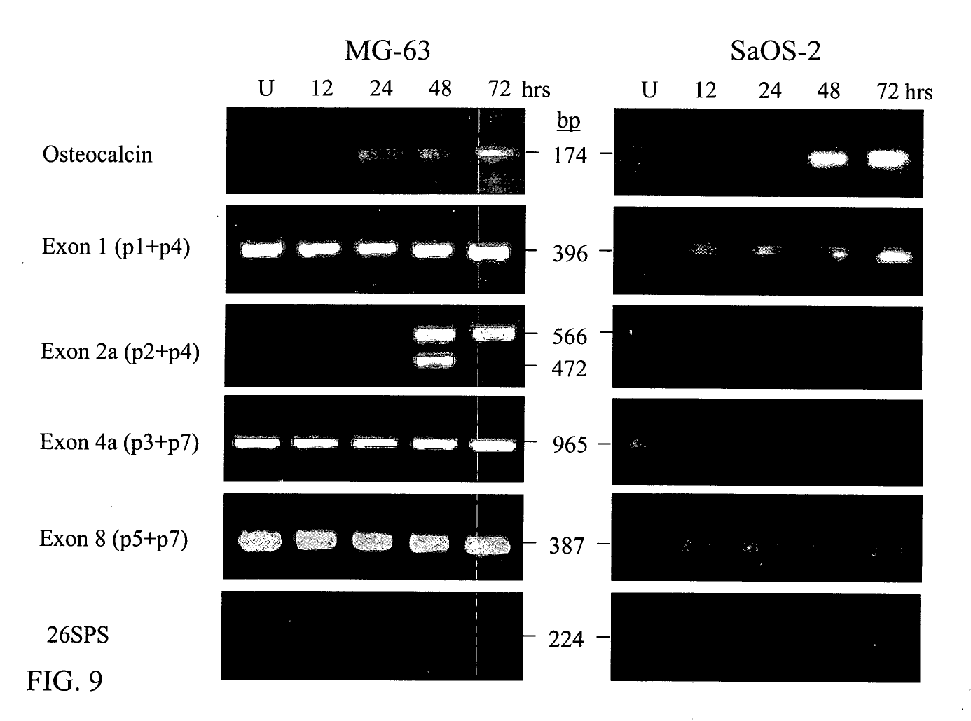

The immunohistochemical analysis, detailed above and in FIG. 5, indicated that the hCHL2 protein is expressed in bone osteoblasts. In order to study the expression of hCHL2 transcripts during osteoblasts differentiation and maturation, the human osteosarcoma cell lines, SaOs-2 (ATCC HTB-85), and MG-63 (ATCC CRL-1427),_ were treated with parathyroid hormone (PTH), a known stimulator of osteoprogenitor differentiation. Osteocalcin, an abundant noncollagenous protein in adult bone matrix, is synthesized and secreted exclusively by mature osteoblasts during the late stage differentiation and mineralization and is thus a marker of bone formation and an indicator of the maturation stage of osteoblastic cell populations. RT-PCR analysis using primers specific for osteocalcin (FIG. 9) showed increased expression in response to PTH, demonstrating that SaOs-2 and MG-63 cells underwent differentiation into mature osteoblasts.

RT-PCR analysis of hCHL2 (FIG. 9) indicates that following PTH treatment of SaOS-2 cells, there is an increase in the expression of transcripts initiating in exons 1 and 2a (Variants I through IX, SEQ ID Nos: 1-9). A high basal level of expression of exon 1-containing transcripts was detected in NG-63 cells prior to addition of PTH, and this did not appear to change considerably upon treatment. However, transcripts initiating in exon 2a show a dramatic increase by 48 hrs following PTH addition to these cells. The variant lacking exon 3 (VAR VII, SEQ ID No: 3) is clearly visible at 48 hrs (FIG. 9). In both cell lines, the transcripts initiating in exon 4a (VAR X, SEQ ID NO: 10) are detected prior to incubation with PTH, and these basal levels appear stable throughout the treatment.

The mouse promyoblast cells (undifferentiatied mesenchymal cells, C2C12, ATCC CRL-1772) were used as an additional model system for muscle and bone differentiation. These cells can be differentiated into myotubes upon incubation with insulin. Alternatively, their differentiation pattern can be shifted to the production of osteoblasts when the cells are incubated in the presence of BMP-2 (Katagiri et al., 1994). RT-PCR analysis using primers specific for myogenin, a marker for muscle differentiation, demonstrated that the C2C12 cells underwent differentiation into myotubes in the presence of insulin (FIG. 10). Expression of myogenin was evident as early as 12 hr after addition of insulin, and not detected in undifferentiated cells or cells treated with BMP-2. A similar analysis using primers specific for osteocalcin, a marker of osteoblasts differentiation, demonstrated that the C2C12 cells underwent differentiation into osteoblasts following incubation with BMP-2 (FIG. 10). Expression of osteocalcin was evident by 24 hr after the addition of BMP-2 and was not apparent in undifferentiated cells. Weak expression of the gene was also visible after 48 and 72 hrs following treatment with insulin.

RT-PCR reactions with different primers of mCHL2 suggest that the murine gene is differentially expressed in osteoblasts, myoblasts and undifferentiated cells (FIG. 10). Furthermore, alternatively spliced variants are expressed differently in muscle and bone. As described previously, expressed sequences of mCHL2 indicate the presence of two alternative first exons, 1a and 1b, encoding for secreted and non-secreted isoforms respectively. In undifferentiated C2C12 cells there is a low basal expression of transcripts initiating in exon 1a or 1b (FIG. 10). Upon addition of insulin or BMP-2 there is an early but brief appearance of transcripts that initiate in exon 1a, while transcripts that initiate in 1b are upregulated in response to insulin, but are not detected after 48 hrs of treatment with insulin or BMPs.

Similarly to hCHL2, exon 3 (containing the second half of CR1) and exon 8 (containing CR3) of mCHL2 are alternative exons. Transcripts that do not contain exon 3 were detected only in undifferentiated cells. Transcripts that include or exclude exon 8 are transcribed in undifferentiated C2C12 cells. There is an immediate but temporary increase of the exon 8-containing transcripts by 12 hrs after addition of insulin or BMP2. By 48 hrs, the levels of all mCHL2 transcripts are decreased below detection levels. A unique transcript, which contains only the second half of exon 8 appears briefly by 24 hrs after treatment with insulin. No equivalent transcript was detected in human cells. To summarize, there is differential expression of hCHL2 and mCHL2 isoforms during myoblast and osteoblast differentiation and maturation.

### EXAMPLE XI

### Differential Expression of the hCHIL2 Variants in Lung, Breast and Prostate Cancers as Compared to Normal Tissues

This Example includes non-limiting, illustrative methods and assays involving the novel hCHL2 variants of the present invention, which shown herein to be useful as markers for lung, breast and prostate cancers that are both sensitive and accurate. These markers were found to be over-expressed in lung, breast and prostate cancers specifically, as opposed to respective normal tissues. The measurement of these markers in patient samples, alone or in combination, provides information that the diagnostician can correlate with a probable diagnosis of lung, breast and prostate cancer. The markers of the present invention, alone or in combination, show a high degree of differential expression between lung, breast and prostate cancer and non-cancerous states.

Hybridization Assays

Detection of a nucleic acid of interest in a biological sample may optionally be effected by hybridization-based assays using an oligonucleotide probe.

Hybridization based assays which allow the detection of a variant of interest (i.e., DNA or RNA) in a biological sample rely on the use of oligonucleotide which can be 10, 15, 20, or 30 to 100 nucleotides long preferably from 10 to 50, more preferably from 40 to 50 nucleotides.

Hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif.] can be attached to the oligonucleotides.

Hybridization assays (or assays with a hybridization component) include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Although the present invention is not specifically dependent on the use of a label for the detection of a particular nucleic acid sequence, such a label might be beneficial, by increasing the sensitivity of the detection.

Furthermore, it enables automation. Probes can be labeled according to numerous well known methods (Sambrook et al., 1989, supra). Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ATP and polynucleotide kinase, using the Klenow fragment of Pol I of E coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Probes of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or deoxyribonucleic acid (DNA), and preferably of DNA.

NAT Assays

Detection of a nucleic acid of interest in a biological sample may also optionally be effected by NAT-based assays, which involve nucleic acid amplification technology, such as PCR for example (or variations thereof such as real-time PCR for example).

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

Polymerase chain reaction (PCR) is carried out in accordance with known techniques, as described for example, in U.S. Pat. Nos. 4,683,195; 47683,202; 4,800,159; and 4,965,188 (the disclosures of all three U.S. patents are incorporated herein by reference). In general, PCR involves a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review of PCR techniques, see PCR Protocols, A Guide to Methods and Amplifications, Michael et al. Eds, Acad. Press, 1990.

As used herein, a “primer” defines an oligonucleotide which is capable of annealing to a target sequence, thereby creating a double stranded region which can serve as an initiation point for DNA synthesis under suitable conditions.

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the 15 particular needs (Walker et al., 1992, Proc. Natl. Acad. Sci. USA 89:392-396; and ibid., 1992, Nucleic Acids Res. 20:1691-1696).

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. Of course, it will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. In general, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The markers of the present invention were tested with regard to their expression in various cancerous and non-cancerous tissue samples. A description of the samples used in the panel is provided in Tables 2-4 below. Tests were then performed as described in Examples XI a-c below.

Materials and Experimental Procedures

RNA preparation—RNA was obtained from Clontech (Franklin Lakes, N.J. USA 07417, www.clontech.com), BioChain Inst. Inc. (Hayward, Calif. 94545 USA www.biochain.com), ABS (Wilmington, Del. 19801, USA, http://www.absbioreagents.com) or Ambion (Austin, Tex. 78744 USA, http://www.ambion.com). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion) and purified using RNeasy columns (Qiagen).

RTPCR—Purified RNA (1 μg) was mixed with 150 ng Random Hexamer primers (Invitrogen) and 500 μM DNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25μl) was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis- cDNA (5 μl), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in-which the reactions achieved a threshold level (Ct) of fluorescence was registered and was used to calculate the relative transcript quantity in the RT reactions. The relative quantity was calculated using the equation Q=efficiency\hat{ }-Ct. The efficiency of the PCR reaction was calculated from a standard curve, created by using serial dilutions of reverse transcription (RT) reactions prepared from RNA purified from 5 cell lines (HCT116, H1299, DU145, MCF7, ES-2). To minimize inherent differences in the RT reaction, the resulting relative quantities were normalized to the geometric mean of the relative quantities of several housekeeping (HSKP) genes.

### EXAMPLE XIa

### Expression of hCHL2 Transcripts which are Detectable by SEQ ID NO:64 in Normal and Cancerous Breast Tissues

Expression of hCHL2 transcripts containing the amplicon of the unique exon 2a as in SEQ ID NO: 64 (e.g., amplicon and unique exon related to variant no. IV) was measured by real time PCR. In parallel the expression of four housekeeping genes—PBGD (GenBank Accession No. BC019323; amplicon—SEQ ID NO: 55), HPRT1 (GenBank Accession No. NM_{—}000194; amplicon—SEQ ID NO: 52), G-6-PD (GenBank Accession No. NM_{—}000402; amplicon—SEQ ID NO: 61) and SDHA (GenBank Accession No. NM_{—}004168; amplicon—SEQ ID NO: 58), was measured similarly. For each RT sample, the expression of SEQ ID NO: 64 was normalized to the geometric mean of the quantities of the housekeeping genes. The normalized quantity of each RT sample was then divided by the median of the quantities of the normal post breast reduction surgery (PS) and PM samples (Sample Nos. 56-60,63-67, Table 2, below), to obtain a value of fold up-regulation for each sample relative to median of the normal PM samples.

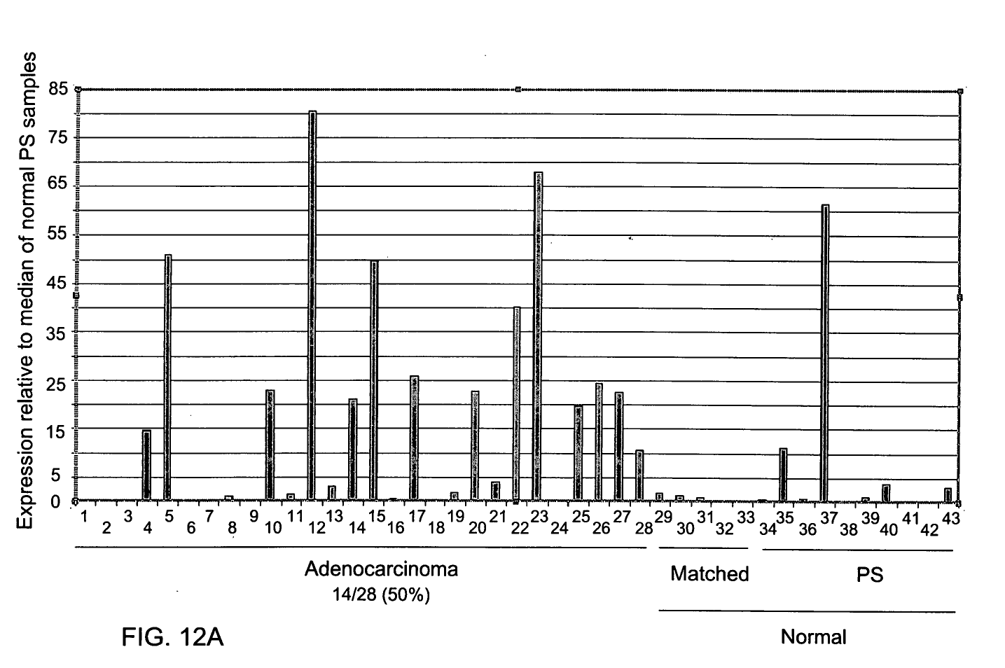

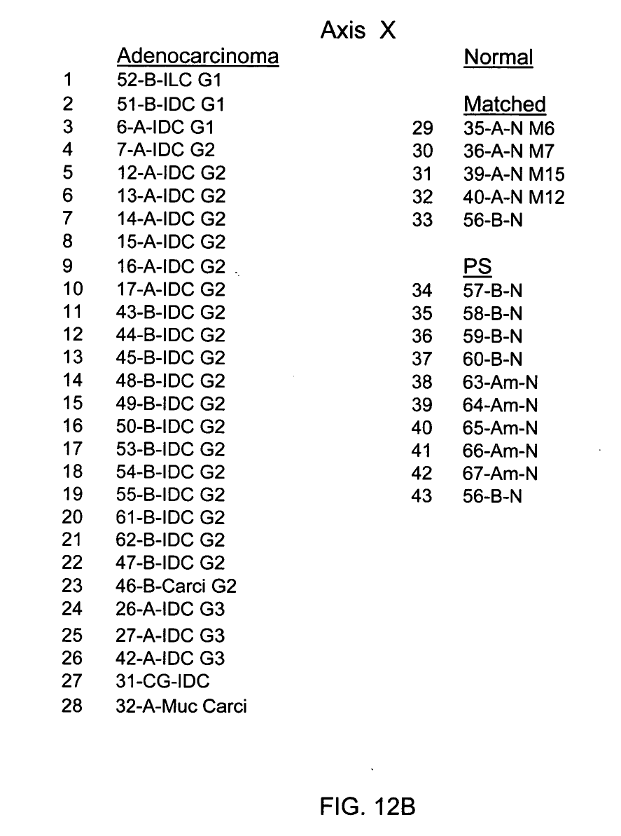

FIG. 12 is a histogram showing over expression of the above-indicated hCHL2 transcripts in cancerous breast samples relative to the normal samples. The number and percentage of cancer samples that exhibit at least 5 fold over-expression, out of the total number of samples tested is indicated in the bottom.

As is evident from FIG. 12, the expression of hCHL2 transcripts containing the amplicon of the unique exon 2a, as in SEQ ID NO: 64, in cancer samples was significantly higher than in the non-cancerous samples. Notably, an over-expression of at least 5 fold was found in 14 out of 28 adenocarcinoma samples.

TABLE 2
Breast panel

| Breast panel | | | | | | | |
|---|---|---|---|---|---|---|---|
| sample | | | | | | | |
| rename | Lot no | source | pathology | grade | age/sex | TNM | stage |
| 52-B-ILC G1 | A605360 | Biochain | Invasive Lobular | 1 | F/60 | | |
| | | | Carcinoma | | | | |
| 51-B-IDC G1 | A605361 | Biochain | IDC | 1 | F/79 | | |
| 6-A-IDC G1 | 7238T | ABS | IDC | 1 | F/60 | T2N0M0 | stage 2A |
| 7-A-IDC G2 | 7263T | ABS | IDC | 2 | F/43 | T1N0M0 | stage 1 |
| 12-A-IDC G2 | 1432T | ABS | IDC | 2 | F/46 | T2N0M0 | stage 2A |
| 13-A-IDC G2 | A0133T | ABS | IDC | 2 | F/63 | T2N1aMx | |
| 14-A-IDC G2 | A0135T | ABS | IDC | 2 | F/37 | T2N2Mx | |
| 15-A-IDC G2 | 7259T | ABS | IDC | 2 | F/59 | T3N1M0 | stage 3A |
| 16-A-IDC G2 | 4904020032T | ABS | IDC | 2 | NA | T3N1Mx | |
| 17-A-IDC G2 | 4904020036T | ABS | IDC | 2-3 | NA | T3N1Mx | |
| 43-B-IDC G2 | A609183 | Biochain | IDC | 2 | F/40 | | |
| 44-B-IDC G2 | A609198 | Biochain | IDC | 2 | F/77 | | |
| 45-B-IDC G2 | A609181 | Biochain | IDC | 2 | F/58 | | |
| 48-B-IDC G2 | A609222 | Biochain | IDC | 2 | F/44 | | |
| 49-B-IDC G2 | A609223 | Biochain | IDC | 2 | F/54 | | |
| 50-B-IDC G2 | A609224 | Biochain | IDC | 2 | F/69 | | |
| 53-B-IDC G2 | A605151 | Biochain | IDC | 2 | F/44 | | |
| 54-B-IDC G2 | A605353 | Biochain | IDC | 2 | F/41 | | |
| 55-B-IDC G2 | A609179 | Biochain | IDC | 2 | F/42 | | |
| 61-B-IDC G2 | A610029 | Biochain | IDC | 2 | F/46 | | |
| 62-B-IDC G2 | A609194 | Biochain | IDC | 2 | F/51 | | |
| 47-B-IDC G2 | A609221 | Biochain | IDC | 2 | | | |
| 46-B-Carci G2 | A609177 | Biochain | Carcinoma | 2 | F/48 | | |
| 26-A-IDC G3 | 7249T | ABS | IDC | 3 | F/60 | T2N0M0 | stage 2A |
| 27-A-IDC G3 | 4907020072T | ABS | IDC | 3 | NA | T2N0Mx | |
| 42-A-IDC G3 | 6005020031T | ABS | IDC | 3 | NA | T1cN0Mx | |
| 31-CG-IDC | CG-154 | Ichilov | IDC | | NA | | |
| 32-A-Muc Carci | 7116T | ABS | Mucinous carcinoma | | F/54 | T2N0M0 | stage 2A |
| 35-A-N M6 | 7238N | ABS | Normal matched to 6T | | F/60 | | |
| 36-A-N M7 | 7263N | ABS | Normal matched to 7T | | F/43 | | |
| 39-A-N M15 | 7259N | ABS | Normal matched to 15T | | F/59 | | |
| 40-A-N M12 | 1432N | ABS | Normal matched to 12T | | F/46 | | |
| 41-A-N M26 | 7249N | ABS | Normal matched to 26T | | F/60 | | |
| 56-B-N | A609235 | Biochain | Normal | | F/59 | | |
| 57-B-N | A609233 | Biochain | Normal | | F/34 | | |
| 58-B-N | A609232 | Biochain | Normal | | F/65 | | |
| 59-B-N | A607155 | Biochain | Normal | | F/35 | | |
| 60-B-N | A609234 | Biochain | Normal | | F/36 | | |
| 63-Am-N | 26486 | Ambion | Normal PS | | F/43 | | |
| 64-Am-N | 23036 | Ambion | Normal PM | | F/57 | | |
| 65-Am-N | 31410 | Ambion | Normal PM | | F/63 | | |
| 66-Am-N | 36678 | Ambion | Normal PM | | F/45 | | |
| 67-Am-N | 073P010602086A | Ambion | Normal PM | | F/64 | | |

According to the present invention, hCHL2 transcripts containing the amplicon of the unique exon 2a as depicted in SEQ ID NO: 64 are a non-limiting example of a marker for diagnosing breast cancer. Although optionally any method may be used to detect overexpression and/or differential expression of this marker, preferably a NAT-based technology is used. Therefore, optionally and preferably, any nucleic acid molecule capable of selectively hybridizing to hCHL2 transcripts detectable by SEQ ID NO: 64 as previously defined is also encompassed within the present invention. Primer pairs are also optionally and preferably encompassed within the present invention; for example, for the above experiment, the following primer pair was used as a non-limiting illustrative example only of a suitable primer pair: exon 2a-forward primer: AACATGGCACTGGTCGGTTT (SEQ ID NO:62); and exon 2a-Reverse primer: CGGACAGTGGAGGCGGTA (SEQ ID NO:63).The present invention also preferably encompasses any amplicon obtained through the use of any suitable primer pair; for example, for the above experiment, the following amplicon was obtained as a non-limiting illustrative example only of a suitable amplicon: exon 2a amplicon (SEQ ID NO:64):

AACATGGCACTGGTCGGTTTGCCAGGCCCAGACATGTTCTGCC
TTTTCCATGGGAAGAGATACTCCCCCGGCGAGAGCTGGCACCCCTAC
TTGGAGCCACAAGGCCTGATGTACTGCCTGCGCTGTACCTGCTCAGA
GGGCGCCCATGTGAGTTGTTACCGCCTCCACTGTCCG.

According to other preferred embodiments of the present invention, hCHL2 transcripts containing exon 2a or a fragment thereof comprise a biomarker for detecting breast cancer. Optionally and more preferably, the fragment of hCHL2 transcripts containing exon 2a comprises unique exon 2a sequence. Also optionally and more preferably, any suitable method may be used for detecting a fragment such as hCHL2 exon 2a for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to hCHL2 transcripts containing exon 2a as described above, including but not limited to SEQ ID NOs: 17, 18, 19, 13, 14 and 15. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

### EXAMPLE XIb

### Expression of hCHL2 Transcripts Which are Detectable by SEQ ID NO:64 in Normal and Cancerous Lung Tissues

Expression of hCHL2 transcripts containing the amplicon of the unique exon 2a as depicted in SEQ ID NO: 64 (e.g., variant no. IV) was measured by real time PCR. In parallel the expression of four housekeeping genes—PBGD (GenBank Accession No. BC019323; amplicon—SEQ ID NO: 55), HPRT1 (GenBank Accession No. NM_{—}000194; amplicon—SEQ ID NO: 52), Ubiquitin (GenBank Accession No. BC000449; amplicon—SEQ ID NO: 67) and SDHA (GenBank Accession No. NM_{—}004168; amplicon—SEQ ID NO: 58), was measured similarly. For each RT sample, the expression of SEQ ID NO: 64 was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 3, below), to obtain a value of fold up-regulation for each sample relative to median of the normal PM samples.

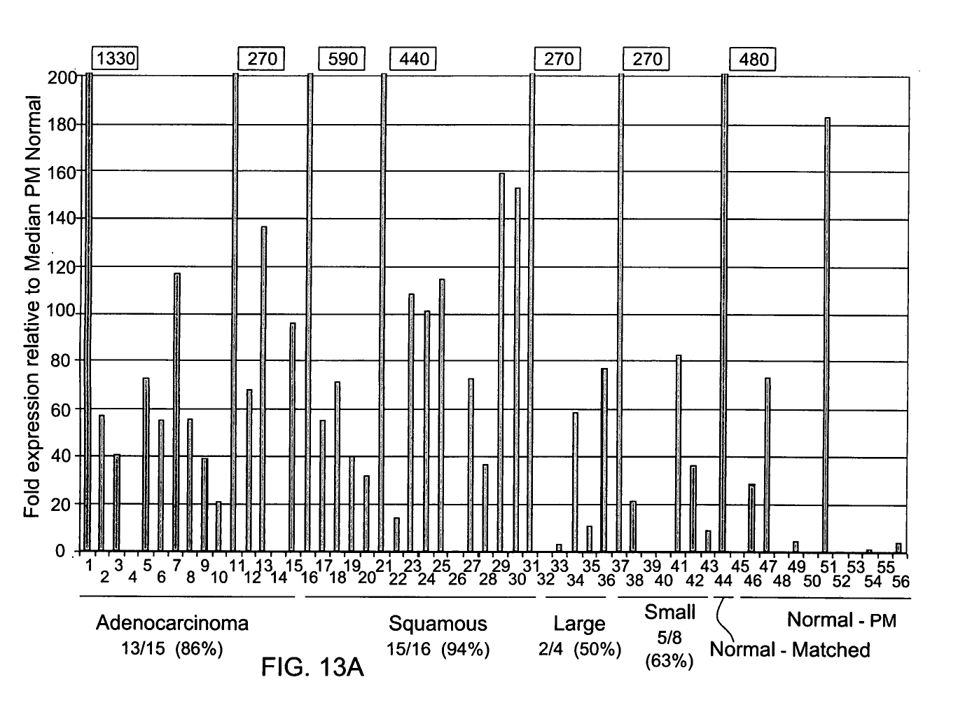

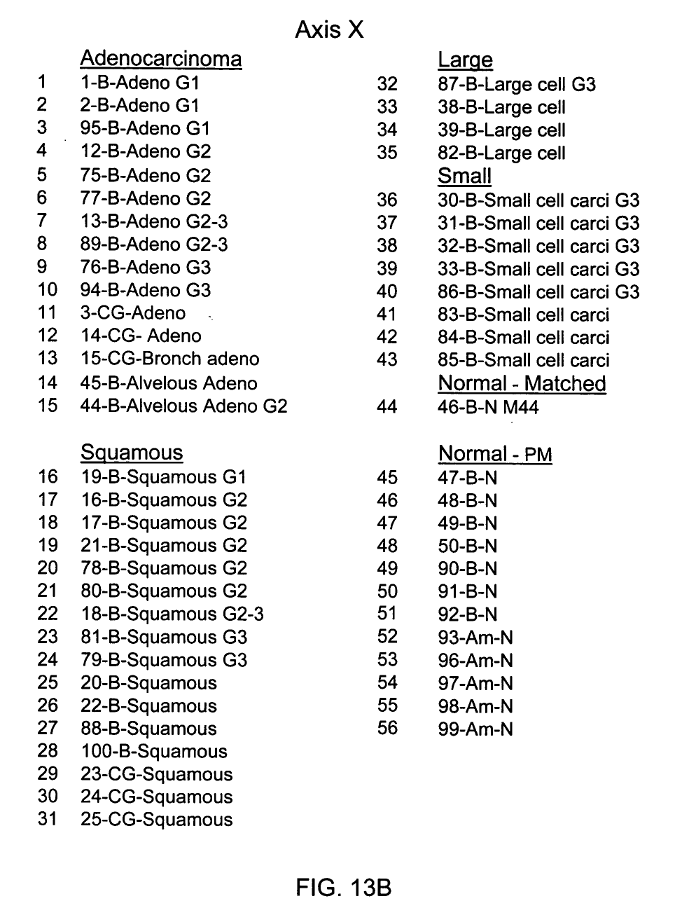

FIG. 13 features a histogram showing over expression of the above-indicated hCHL2 transcripts in cancerous lung samples relative to the normal samples. The number and percentage of cancer samples that exhibit at least 10 fold over-expression, out of the total number of samples tested is indicated in the bottom.

As is evident from FIG. 13, the expression of hCHL2 transcripts detectable by SEQ ID NO: 64 in cancer samples was significantly higher than in the non-cancerous samples. Notably an over-expression of at least 10 fold was found in 13 out of 15 adenocarcinoma samples, 15 out of 16 squamous cell carcinoma samples, 2 out of 4 large cell carcinoma samples and in 5 out of 8 small-cell carcinoma samples.

TABLE 3
Lung panel

| | | | | G | |
|---|---|---|---|---|---|
| sample rename | Lot No. | source | Pathology | Grade | Gender/age |
| 1-B-Adeno G1 | A504117 | Biochain | Adenocarcinoma | 1 | F/29 |
| 2-B-Adeno G1 | A504118 | Biochain | Adenocarcinoma | 1 | M/64 |
| 95-B-Adeno G1 | A610063 | Biochain | Adenocarcinoma | 1 | F/54 |
| 12-B-Adeno G2 | A504119 | Biochain | Adenocarcinoma | 2 | F/74 |
| 75-B-Adeno G2 | A609217 | Biochain | Adenocarcinoma | 2 | M/65 |
| 77-B-Adeno G2 | A608301 | Biochain | Adenocarcinoma | 2 | M/44 |
| 13-B-Adeno G2-3 | A504116 | Biochain | Adenocarcinoma | 2-3 | M/64 |
| 89-B-Adeno G2-3 | A609077 | Biochain | Adenocarcinoma | 2-3 | M/62 |
| 76-B-Adeno G3 | A609218 | Biochain | Adenocarcinoma | 3 | M/57 |
| 94-B-Adeno G3 | A610118 | Biochain | Adenocarcinoma | 3 | M/68 |
| 3-CG-Adeno | CG-200 | Ichilov | Adenocarcinoma | | NA |
| 14-CG-Adeno | CG-111 | Ichilov | Adenocarcinoma | | M/68 |
| 15-CG-Bronch adeno | CG-244 | Ichilov | Bronchioloalveolar | | M/74 |
| | | | adenocarcinoma | | |
| 45-B-Alvelous Adeno | A501221 | Biochain | Alveolus carcinoma | | F/50 |
| 44-B-Alvelous Adeno G2 | A501123 | Biochain | Alveolus carcinoma | 2 | F/61 |
| 19-B-Squamous G1 | A408175 | Biochain | Squamous carcinoma | 1 | M/78 |
| 16-B-Squamous G2 | A409091 | Biochain | Squamous carcinoma | 2 | F/68 |
| 17-B-Squamous G2 | A503183 | Biochain | Squamous carcinoma | 2 | M/57 |
| 21-B-Squamous G2 | A503187 | Biochain | Squamous carcinoma | 2 | M/52 |
| 78-B-Squamous G2 | A607125 | Biochain | Squamous Cell Carcinoma | 2 | M/62 |
| 80-B-Squamous G2 | A609163 | Biochain | Squamous Cell Carcinoma | 2 | M/74 |
| 18-B-Squamous G2-3 | A503387 | Biochain | Squamous Cell Carcinoma | 2-3 | M/63 |
| 81-B-Squamous G3 | A609076 | Biochain | Squamous Carcinoma | 3 | m/53 |
| 79-B-Squamous G3 | A609018 | Biochain | Squamous Cell Carcinoma | 3 | M/67 |
| 20-B-Squamous | A501121 | Biochain | Squamous Carcinoma | | M/64 |
| 22-B-Squamous | A503386 | Biochain | Squamous Carcinoma | | M/48 |
| 88-B-Squamous | A609219 | Biochain | Squamous Cell Carcinoma | | M/64 |
| 100-B-Squamous | A409017 | Biochain | Squamous Carcinoma | | M/64 |
| 23-CG-Squamous | CG-109 (1) | Ichilov | Squamous Carcinoma | | M/65 |
| 24-CG-Squamous | CG-123 | Ichilov | Squamous Carcinoma | | M/76 |
| 25-CG-Squamous | CG-204 | Ichilov | Squamous Carcinoma | | M/72 |
| 87-B-Large cell G3 | A609165 | Biochain | Large Cell Carcinoma | 3 | F/47 |
| 38-B-Large cell | A504113 | Biochain | Large cell | | M/58 |
| 39-B-Large cell | A504114 | Biochain | Large cell | | F/35 |
| 82-B-Large cell | A609170 | Biochain | Large Cell Neuroendocrine | | M/68 |
| | | | Carcinoma | | |
| 30-B-Small cell carci G3 | A501389 | Biochain | small cell | 3 | M/34 |
| 31-B-Small cell carci G3 | A501390 | Biochain | small cell | 3 | F/59 |
| 32-B-Small cell carci G3 | A501391 | Biochain | small cell | 3 | M/30 |
| 33-B-Small cell carci G3 | A504115 | Biochain | small cell | 3 | M |
| 86-B-Small cell carci G3 | A608032 | Biochain | Small Cell Carcinoma | 3 | F/52 |
| 83-B-Small cell carci | A609162 | Biochain | Small Cell Carcinoma | | F/47 |
| 84-B-Small cell carci | A609167 | Biochain | Small Cell Carcinoma | | F/59 |
| 85-B-Small cell carci | A609169 | Biochain | Small Cell Carcinoma | | M/66 |
| 46-B-N M44 | A501124 | Biochain | Normal M44 | | F/61 |
| 47-B-N | A503205 | Biochain | Normal PM | | M/26 |
| 48-B-N | A503206 | Biochain | Normal PM | | M/44 |
| 49-B-N | A503384 | Biochain | Normal PM | | M/27 |
| 50-B-N | A503385 | Biochain | Normal PM | | M/28 |
| 90-B-N | A608152 | Biochain | Normal (Pool 2) PM | | pool 2 |
| 91-B-N | A607257 | Biochain | Normal (Pool 2) PM | | pool 2 |
| 92-B-N | A503204 | Biochain | Normal PM | | m/28 |
| 93-Am-N | 111P0103A | Ambion | Normal PM | | F/61 |
| 96-Am-N | 36853 | Ambion | Normal PM | | F/43 |
| 97-Am-N | 36854 | Ambion | Normal PM | | M/46 |
| 98-Am-N | 36855 | Ambion | Normal PM | | F/72 |
| 99-Am-N | 36856 | Ambion | Normal PM | | M/31 |

According to the present invention, hCHL2 transcripts containing the amplicon of the unique exon 2a as depicted in SEQ ID NO: 64 are non-limiting examples of a marker for diagnosing lung cancer. Although optionally any method may be used to detect overexpression and/or differential expression of this marker, preferably a NAT-based technology is used. Therefore, optionally and preferably, any nucleic acid molecule capable of selectively hybridizing to hCHL2 transcripts containing the sequence of the amplicon, as depicted in SEQ ID NO: 64 as previously defined is also encompassed within the present invention. Primer pairs are also optionally and preferably encompassed within the present invention; for example, for the above experiment, the following primer pair was used as a non-limiting illustrative example only of a suitable primer pair: exon 2a-forward primer: AACATGGCACTGGTCGGTTT (SEQ ID NO:62); and exon 2a-Reverse primer:

| | |
|---|---|
| CGGACAGTGGAGGCGGTA. | (SEQ ID NO:63) |

The present invention also preferably encompasses any amplicon obtained through the use of any suitable primer pair; for example, for the above experiment, the following amplicon was obtained as a non-limiting illustrative example only of a suitable amplicon: exon 2a amplicon (SEQ ID NO:64):

AACATGGCACTGGTCGGTTTGCCAGGCCCAGACATGTTCTGC
CTTTTCCATGGGAAGAGATACTCCCCCGGCGAGAGCTGGCACCCCT
ACTTGGAGCCACAAGGCCTGATGTACTGCCTGCGCTGTACCTGCTCA
GAGGGCGCCCATGTGAGTTGTTACCGCCTCCACTGTCCG.

According to other preferred embodiments of the present invention, hCHL2 transcripts containing exon 2a or a fragment thereof comprises a biomarker for detecting lung cancer. Optionally and more preferably, the fragment of hCHL2 transcripts containing exon 2a comprises unique exon 2a sequence. Also optionally and more preferably, any suitable method may be used for detecting a fragment such as hCHL2 exon 2a for example. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to hCHL2 transcripts containing exon 2a as described above, including but not limited to SEQ ID NOs: 17, 18, 19, 13, 14 and 15. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

### EXAMPLE XIc

### Expression of hCHL2 Transcripts which are Detectable by SEQ ID NO:70 in Normal, Benign and Cancerous Prostate Tissues

Expression of hCHL2 transcripts containing the amplicon of the unique exon 4a, as in SEQ ID NO:70 (e.g., amplicon and unique exon related to variant no. X), was measured by real time PCR. In parallel the expression of four housekeeping genes - PBGD (GenBank Accession No. BC019323; amplicon—SEQ ID NO: 55), HPRT1 (GenBank Accession No. NM_{—}000194; amplicon—SEQ ID NO: 52), RPL19 (GenBank Accession No. NM_{—}000981; amplicon—SEQ ID NO: 73) and SDHA (GenBank Accession No. NM_{—}004168; amplicon—SEQ ID NO: 58), was measured similarly. For each RT sample, the expression of SEQ ID NO: 70 was normalized to the geometric mean of the quantities of the housekeeping genes. The normalized quantity of each RT sample was then divided by the median of the quantities of the normal post-mortem (PM) samples (Sample Nos. 42, 48-53, 59-63, Table 4, below), to obtain a value of fold up-regulation for each sample relative to median of the normal PM samples.

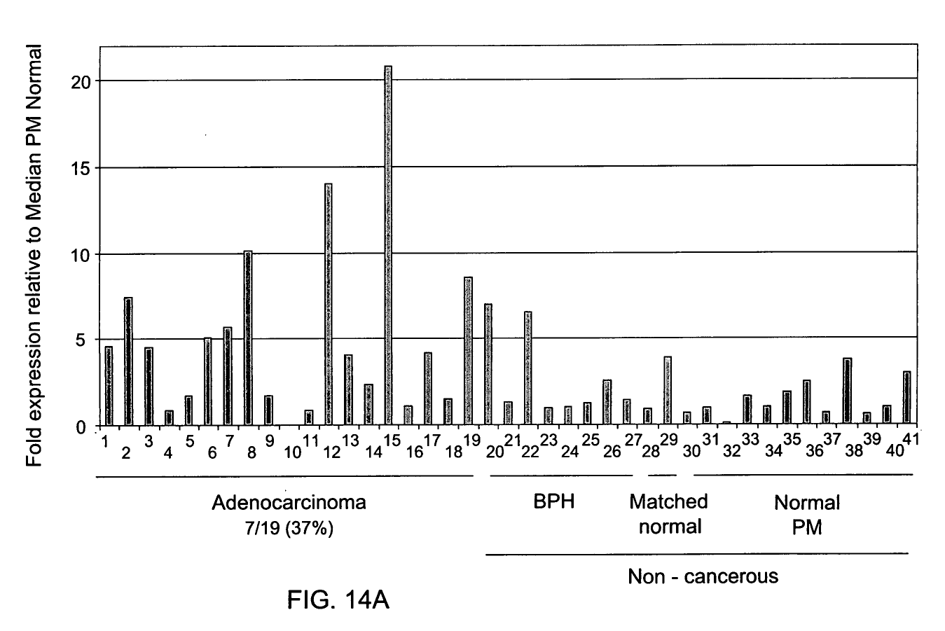

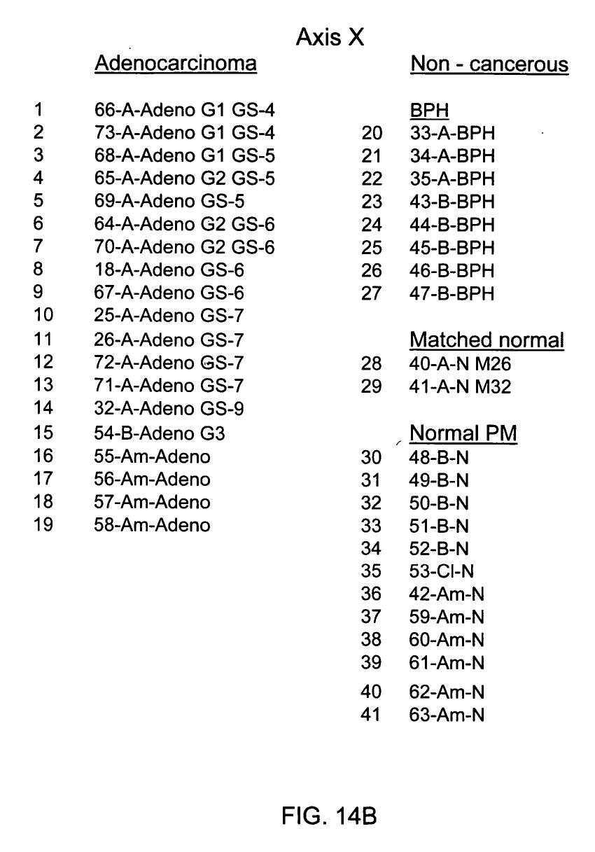

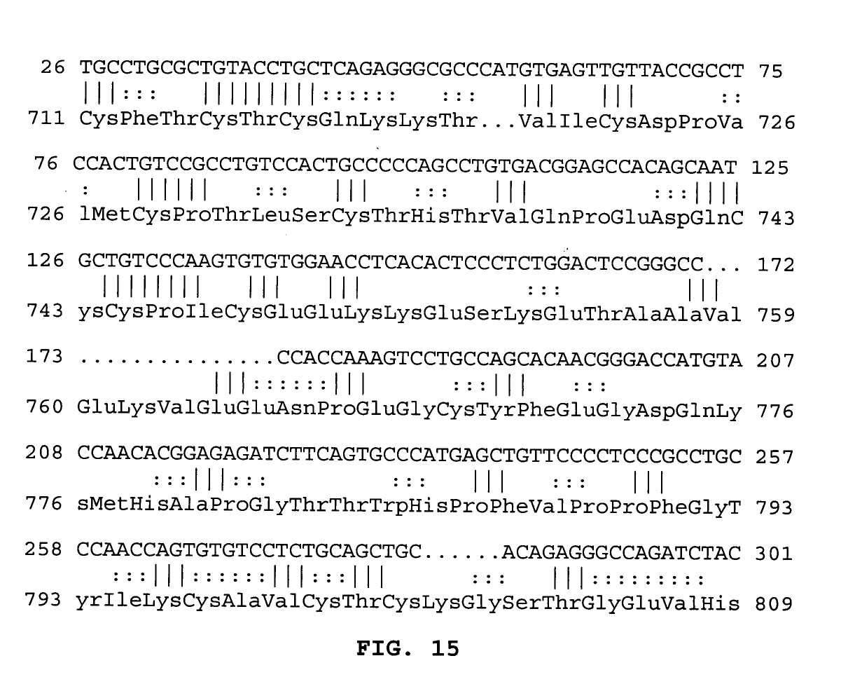

FIG. 14 is a histogram showing over expression of the above-indicated hCHL2 transcripts in cancerous and benign prostate samples relative to the normal samples. The number and percentage of cancer samples that exhibit at least 5 fold over-expression, out of the total number of samples tested is indicated in the bottom.

As is evident from FIG. 14, the expression of hCHL2 transcripts containing the amplicon of the unique exon 4a, as in SEQ ID NO: 70, in cancer samples was significantly higher than in the non-cancerous samples. Notably, an over-expression of at least 5 fold was found in 7 out of 19 adenocarcinoma samples. An over-expression of 5-7 fold was observed also in 2 of the 8 BPH samples.

TABLE 4

| | Lot No. | pathology | Sex/Age | Source |
|---|---|---|---|---|
| 66-A-Adeno G1 GS-4 | 160202 | Adenocarcinoma Gleason score 4 | M/64 | ABS |
| 73-A-Adeno G1 GS-4 | 16026T2 | Acinar Adenocarcinoma Gleason score 4 (2 + 2) | M/77 | ABS |
| 68-A-Adeno G1 GS-5 | 160172 | Adenocarcinoma Gleason score 5 | M/66 | ABS |
| 56-Am-Adeno G1 GS-5 | 36467 | Adenocarcinoma, Gleason score 5 (3 + 2); stage 2 | M/72 | Ambion |
| 58-Am-Adeno G1 GS-5 | 37192 | Adenocarcinoma, Gleason score 5; stage 2 | M/52 | Ambion |
| 65-A-Adeno G2 GS-5 | 160022 | Adenocarcinoma Gleason score 5 | M/66 | ABS |
| 69-A-Adeno GS-5 | 160182 | Acinar Adenocarcinoma Gleason score 5 | M/58 | ABS |
| 55-Am-Adeno GS-5 | 36464 | Adenocarcinoma, Gleason score 5; stage 1 | M/53 | Ambion |
| 64-A-Adeno G2 GS-6 | 160092 | Acinar Adenocarcinoma Gleason score 6 | M/71 | ABS |
| 70-A-Adeno G2 GS-6 | 160192 | Adenocarcinoma Gleason score 6 | M/53 | ABS |
| 18-A-Adeno GS-6 | 5610020069T | Adenocarcinoma, Gleason score 6 (3 + 3) | M | ABS |
| 67-A-Adeno GS-6 | 160142 | Acinar Adenocarcinoma Gleason score 6 | M/62 | ABS |
| 25-A-Adeno GS-7 | 5605020052T | Adenocarcinoma, Gleason score 7 (4 + 3) | M | ABS |
| 26-A-Adeno GS-7 | 5609020067T | Adenocarcinoma, Gleason score 7 (4 + 3) | M | ABS |
| 72-A-Adeno GS-7 | 160122 | Acinar Adenocarcinoma Gleason score 7 | M/66 | ABS |
| 71-A-Adeno GS-7 | 160242 | Acinar Adenocarcinoma Gleason score 7 | M/70 | ABS |
| 57-Am-Adeno GS-7 | 26442 | Adenocarcinoma, Gleason score 7 | M/62 | Ambion |
| 32-A-Adeno GS-9 | 5604020042T | Adenocarcinoma, Gleason score 9 (5 + 4) | M | ABS |
| 54-B-Adeno G3 | A610031 | Adenocarcinoma | | Biochain |
| 33-A-BPH | 5607020058 | BPH | M | ABS |
| 34-A-BPH | 5607020059 | BPH | M | ABS |
| 35-A-BPH | 5607020060 | BPH | M | ABS |
| 43-B-PBH | A609267 | BPH | M/66 | Biochain |
| 44-B-PBH | A609268 | BPH | M/72 | Biochain |
| 45-B-PBH | A609269 | BPH | M/69 | Biochain |
| 46-B-PBH | A609270 | BPH | M/65 | Biochain |
| 47-B-PBH | A609271 | BPH | M/71 | Biochain |
| 40-A-N M26 | 5609020067N | Normal Matched | M | ABS |
| 41-A-N M32 | 5604020042N | Normal Matched | M | ABS |
| 48-B-N | A609257 | Normal PM | M/24 | Biochain |
| 49-B-N | A609256 | Normal PM | M/36 | Biochain |
| 50-B-N | A609255 | Normal PM | M/26 | Biochain |
| 51-B-N | A609258 | Normal PM | M/27 | Biochain |
| 52-B-N | A609254 | Normal PM | M/29 | Biochain |
| 53-Cl-N | 1070317 | Normal - Pool of 47 | M | Clontech |
| 42-Am-N | 061P04A | Normal (IC BLEED) | M/47 | ambion |
| 59-Am-N | 25955 | Normal PM (Head trauma) | M/62 | Ambion |
| 60-Am-N | 33605 | Normal PM (Myocardial infraction) | M/69 | Ambion |
| 61-Am-N | 34077 | Normal PM (Alzheimer's) | M/71 | Ambion |
| 62-Am-N | 31316 | Normal (Renal failure) | M/79 | Ambion |
| 63-Am-N | 30991 | Normal (Gall Bladder cancer) | M/78 | Ambion |

According to the present invention, hCHL2 transcripts containing the amplicon of the unique exon 4a, as in SEQ ID NO: 70, are a non-limiting example of a marker for diagnosing prostate cancer. Although optionally any method may be used to detect overexpression and/or differential expression of this marker, preferably a NAT-based technology is used. Therefore, optionally and preferably, any nucleic acid molecule capable of selectively hybridizing to hCHL2 transcripts detectable by SEQ ID NO: 70 as previously defined is also encompassed within the present invention. Primer pairs are also optionally and preferably encompassed within the present invention; for example, for the above experiment, the following primer pair was used as a non-limiting illustrative example only of a suitable primer pair: exon 4a-forward primer: AAACCTCATTTTCTTCTTCCTCCTG (SEQ ID NO:68); and exon 4a-Reverse primer:

| | |
|---|---|
| CTGAAGATCTCTCCGTGTTGGTACATG. | (SEQ ID NO:69) |

The present invention also preferably encompasses any amplicon obtained through the use of any suitable primer pair; for example, for the above experiment, the following amplicon was obtained as a non-limiting illustrative example only of a suitable amplicon: exon 4a amplicon (SEQ ID NO:70):

AAACCTCATTTTCTTCTTCCTCCTGCCCCTCCCCCA
CTGCAGAACCTCACACTCCCTCTGGACTCCGGGCCCCACCAAAGTCC
TgCCAGCACAACGGGACCATGTACCAACACGGA GAGATCTTCAG.

The present invention also optionally and preferably includes any peptide and amino acid sequence corresponding to any of the above nucleotide sequences, and/or any epitopes as previously described. According to still other preferred embodiments, the present invention optionally and preferably encompasses any amino acid sequence or fragment thereof encoded by a nucleic acid sequence corresponding to hCHL2 transcripts containing exon 4a as described above, including but not limited to SEQ ID NO:20 or a portion thereof. Any oligopeptide or peptide relating to such an amino acid sequence or fragment thereof may optionally also (additionally or alternatively) be used as a biomarker. The present invention also optionally encompasses antibodies capable of recognizing, and/or being elicited by, such an oligopeptide or peptide.

### EXAMPLE XII

### CHL2 Splice Variants of Group II

The novel CHL2 is a homolog to the known chordins within the VWFC domain, named after the von-Willebrand factor (VWF) type C repeat, which is found 2-4 times in these multi-domain proteins. The VWF domain has a length of about 70 amino acids covering 10 well conserved cysteines. The presence of this region in complex-forming proteins leads to the assumption that the VWFC domain might be involved in forming larger protein complexes. The various variants to the full sequence (SEQ ID NO: 74-84) have 2, 3 or 4 VWF type repeats. SEQ ID NO. 75 and 79 also has a sequence coding for a signal sequence, while SEQ ID NO: 76 and 77 are predicted not to have such a signal sequence.

Thus the present invention provides by its first aspect, a novel isolated nucleic acid molecule comprising or consisting of the sequence of any one of SEQ ID NO: 74 to SEQ ID NO: 84, fragments of said sequence having at least 20 nucleic acids, or a molecule comprising a sequence having at least 70%, preferably 80%, and most preferably 90% or 95% identity to any one of SEQ ID NO:74 to SEQ ID NO: 84.

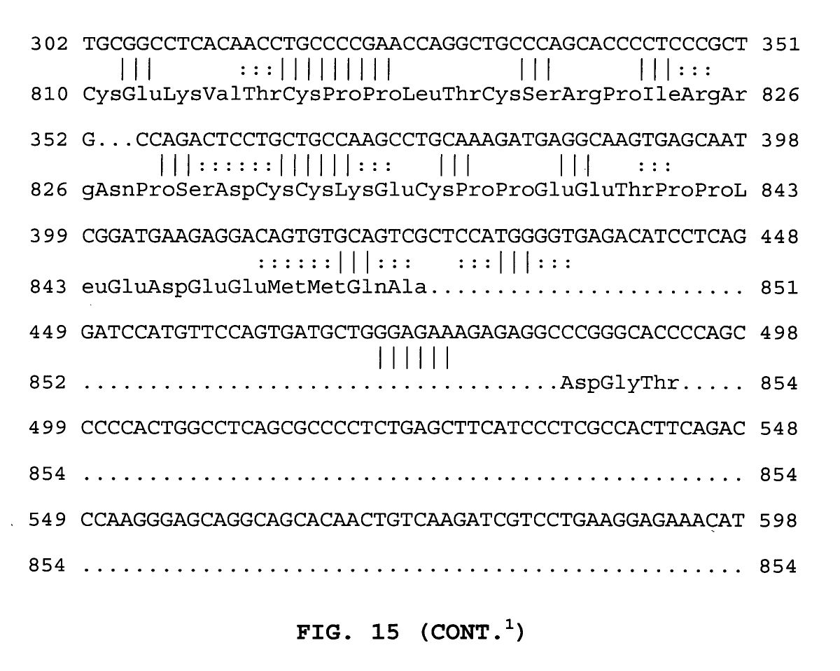

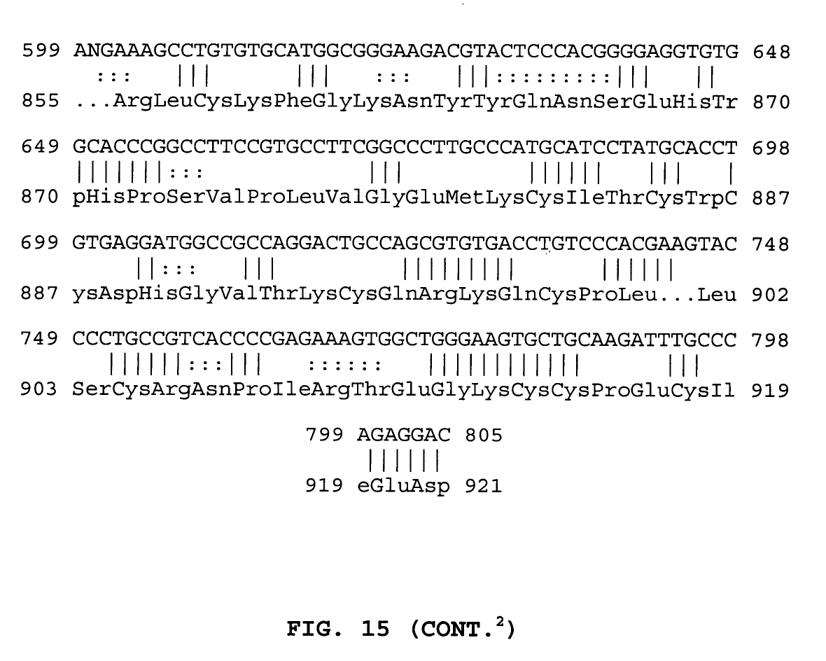

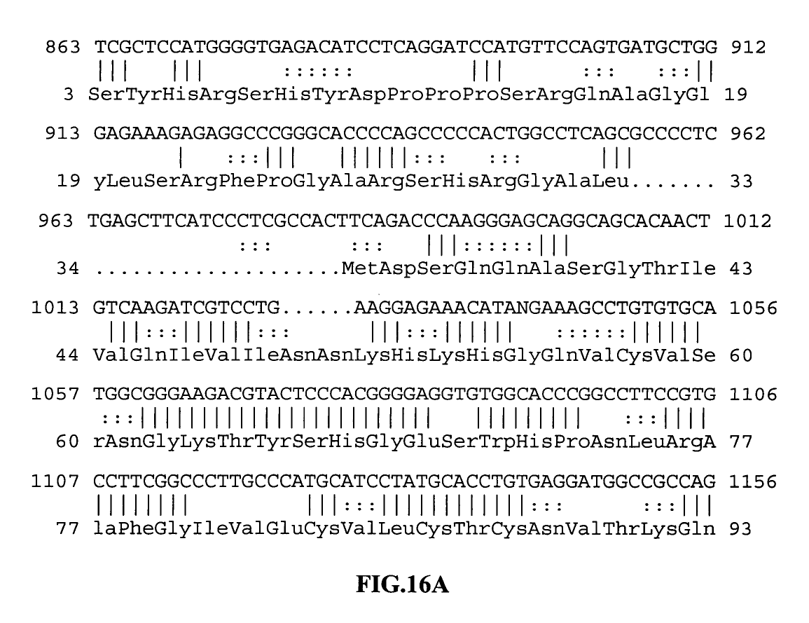

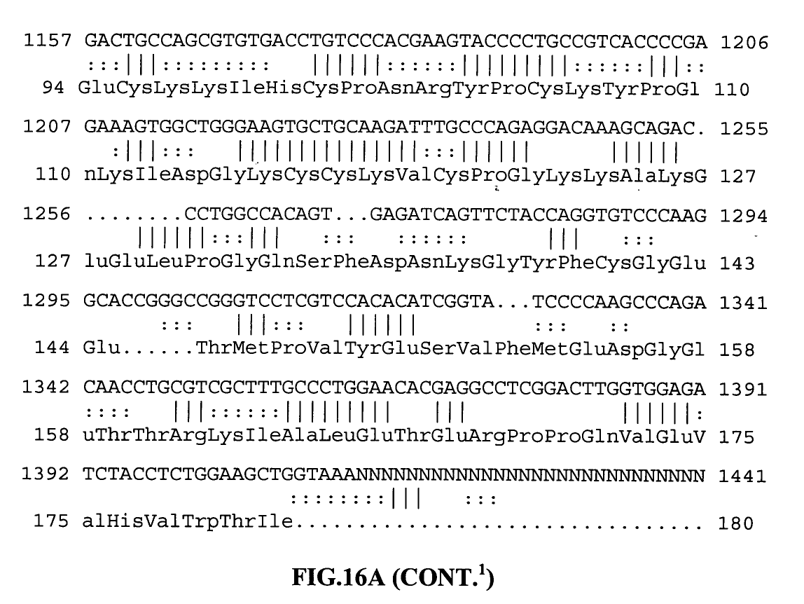

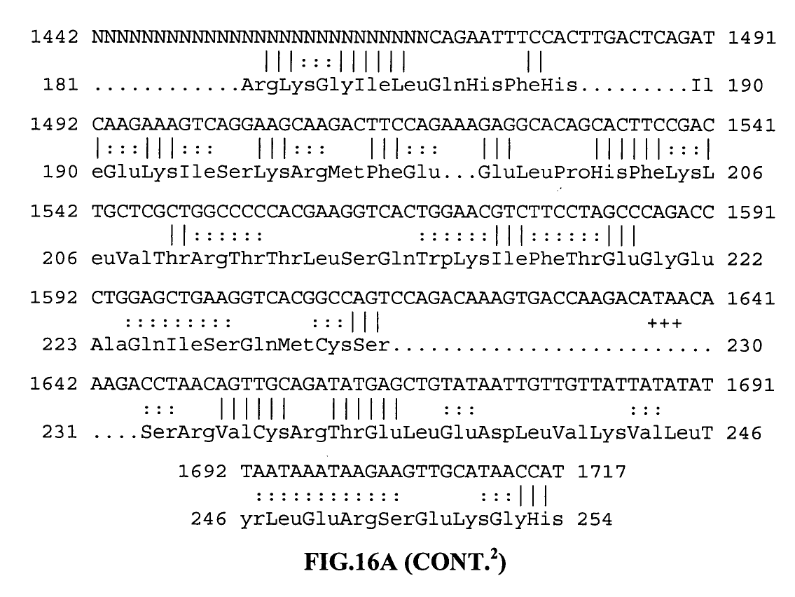

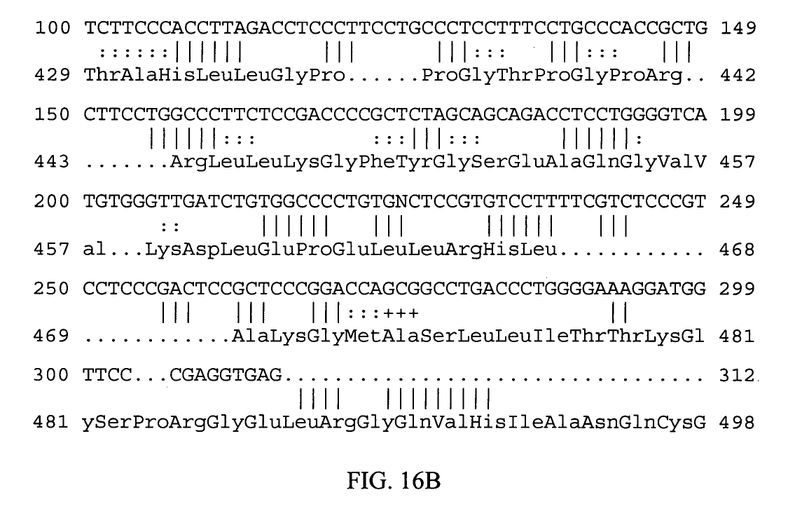

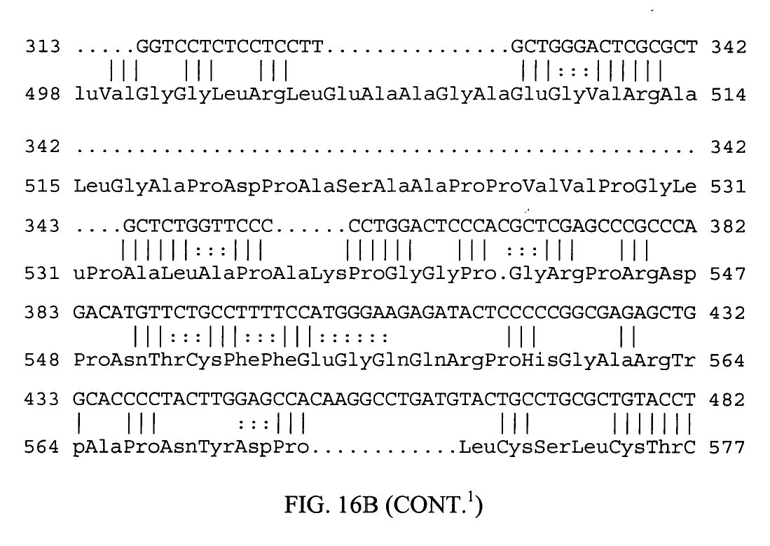

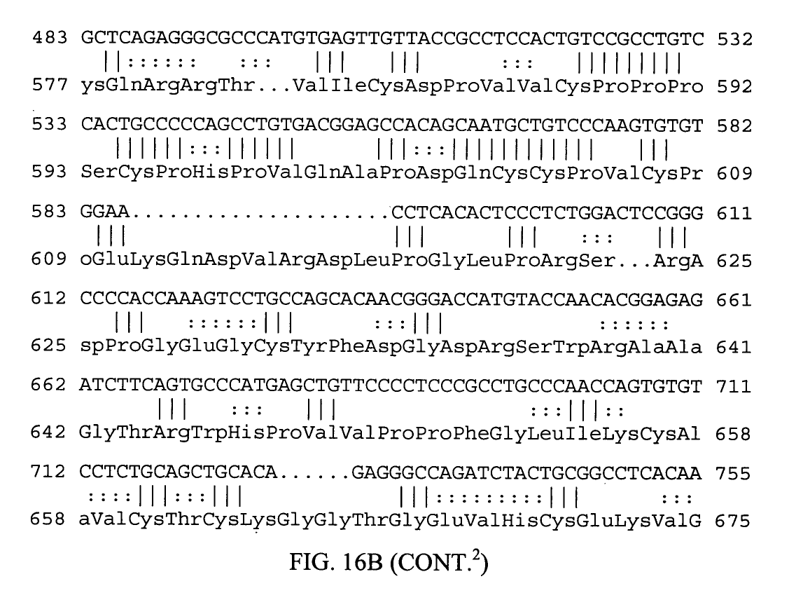

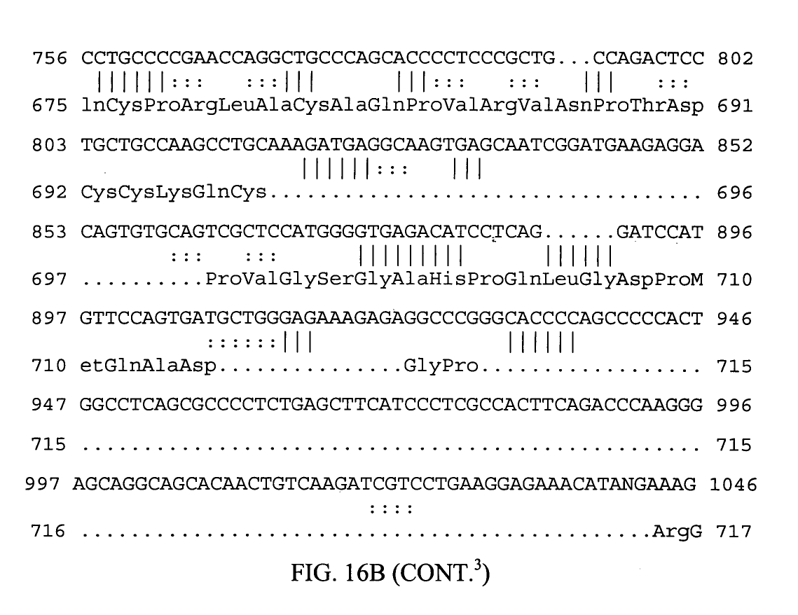

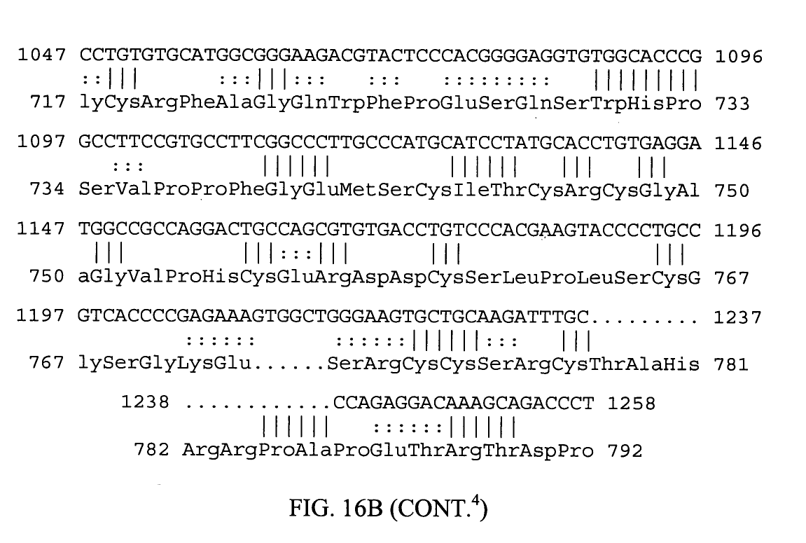

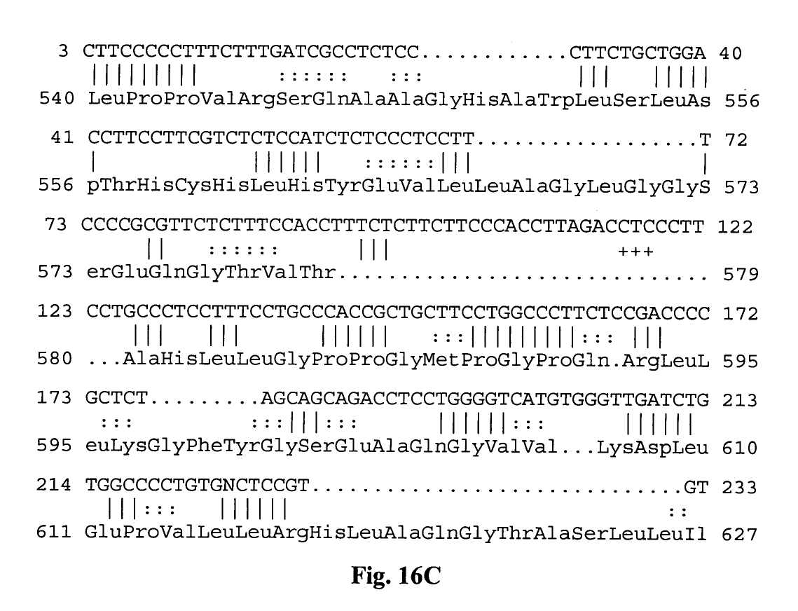

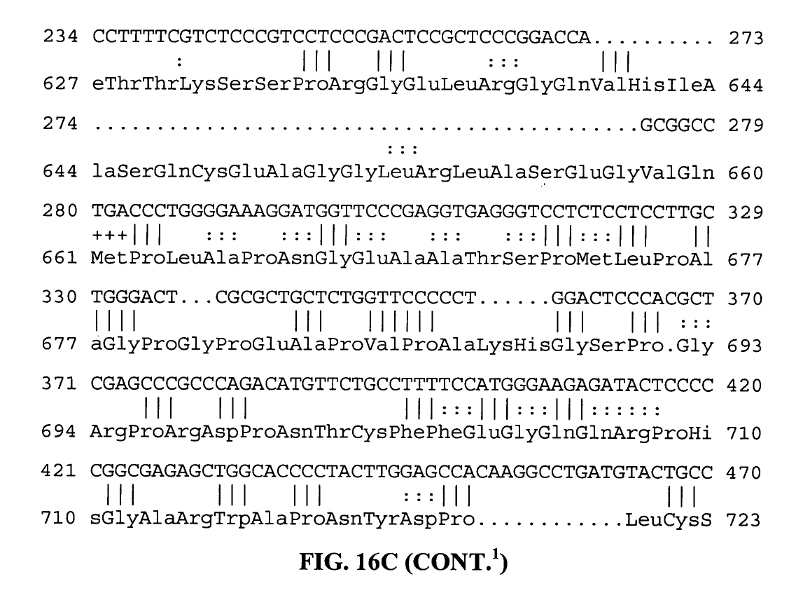

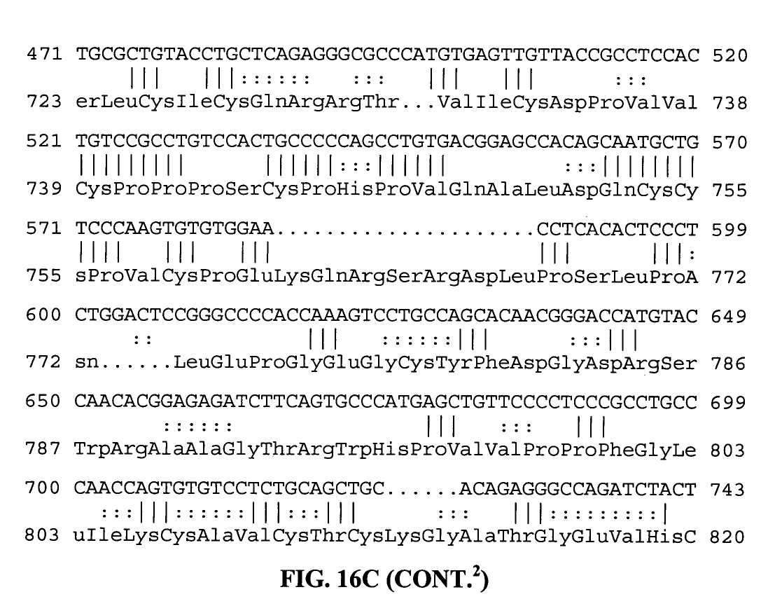

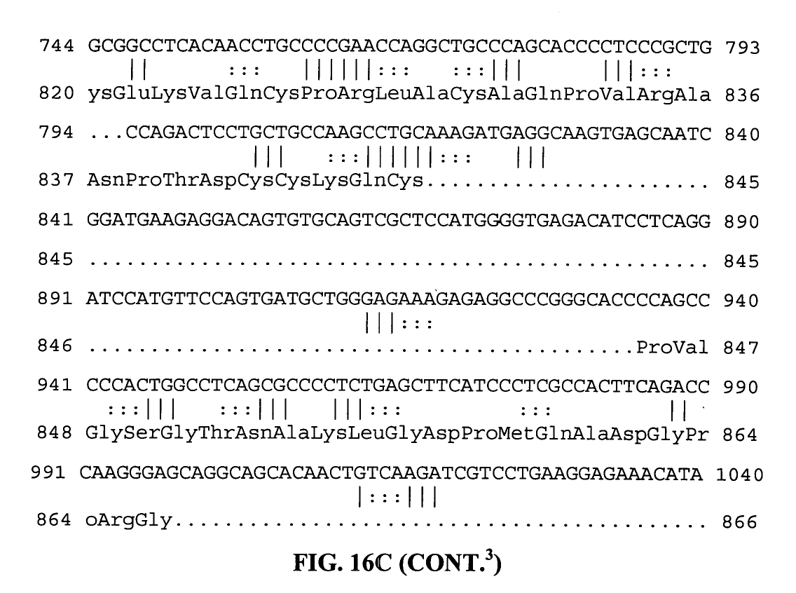

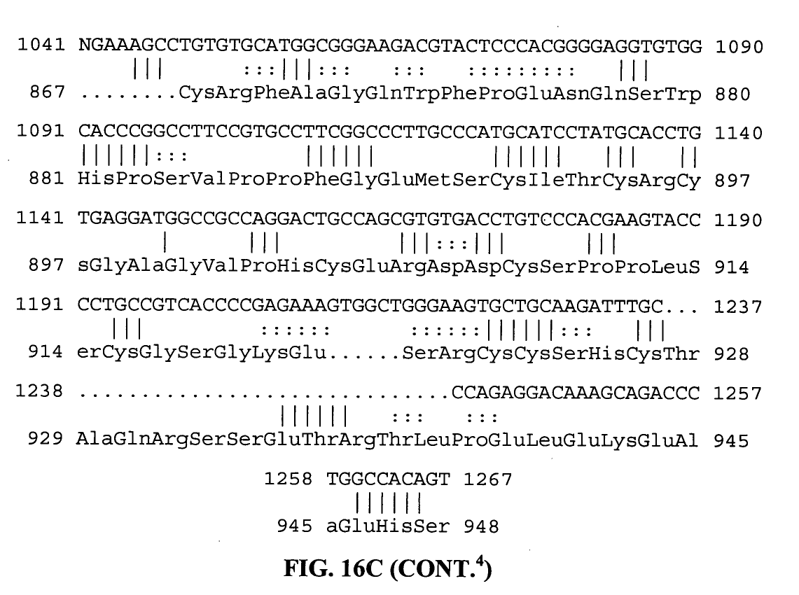

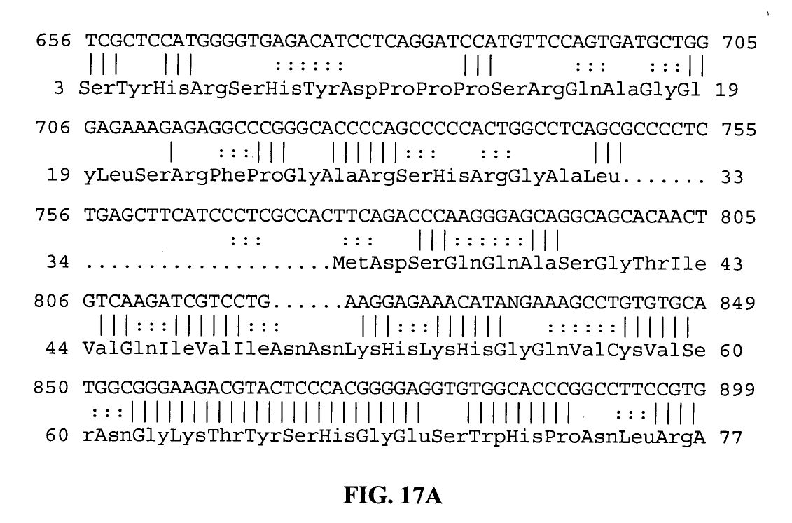

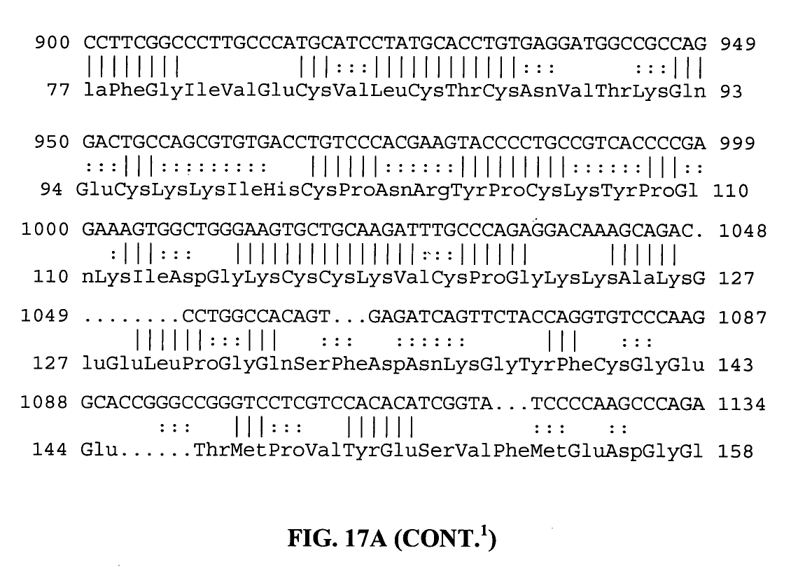

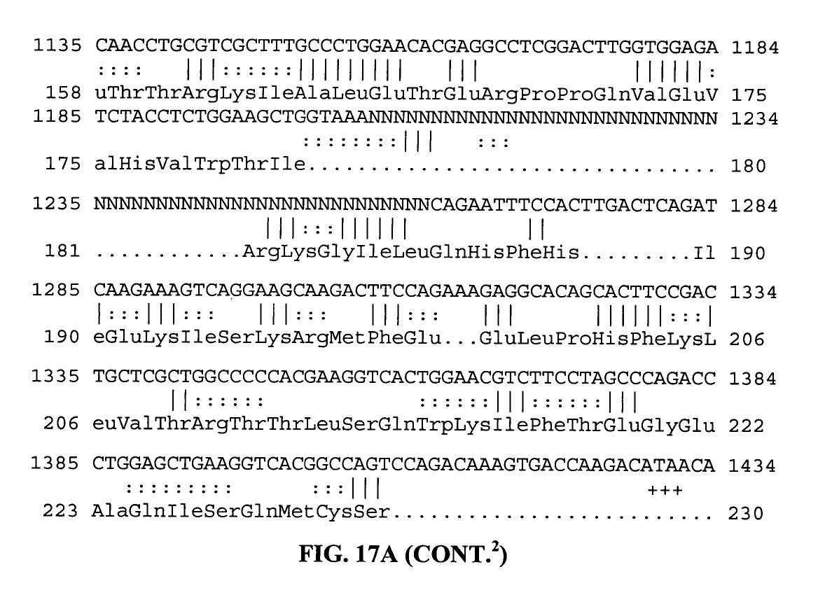

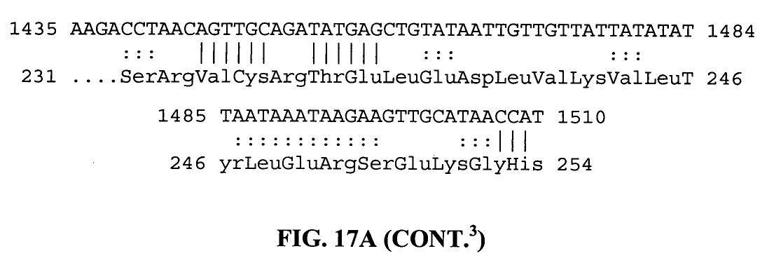

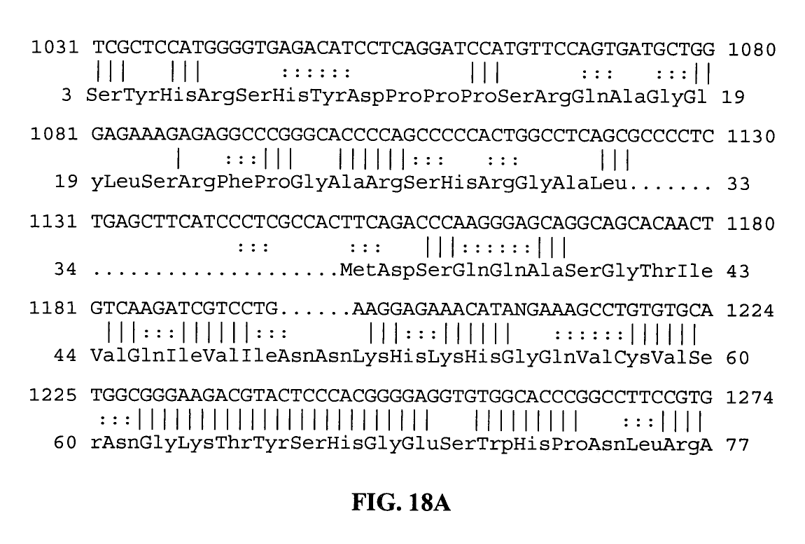

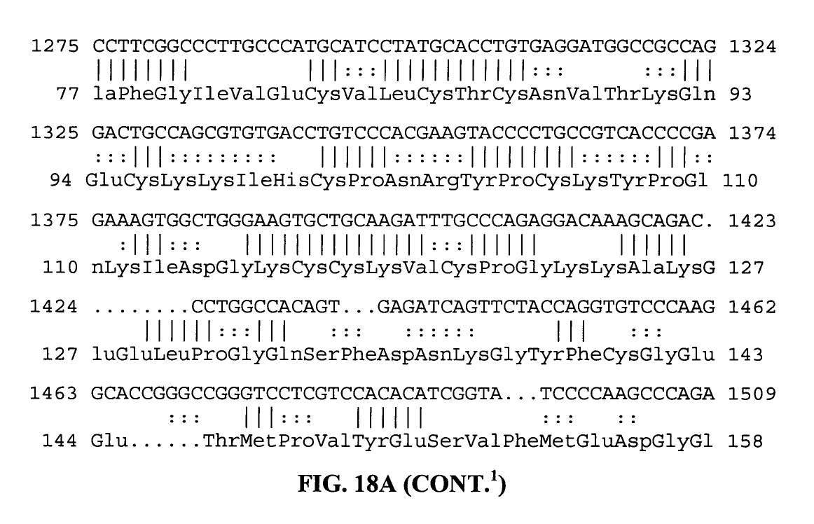

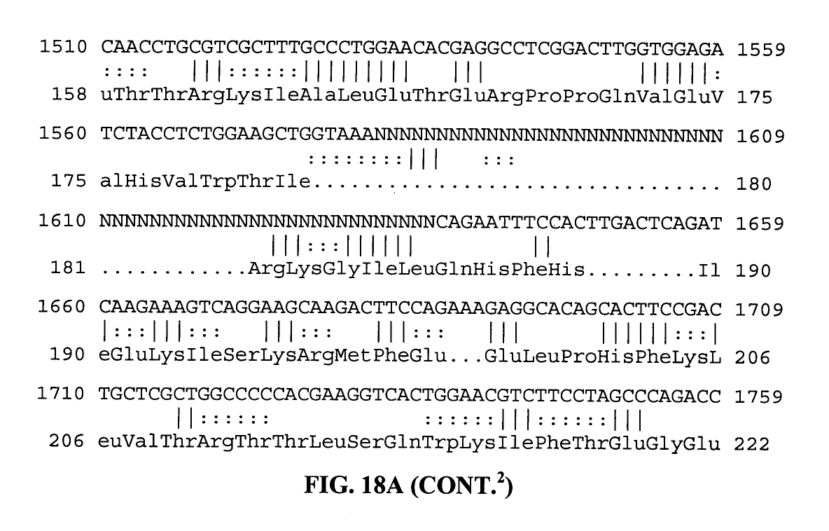

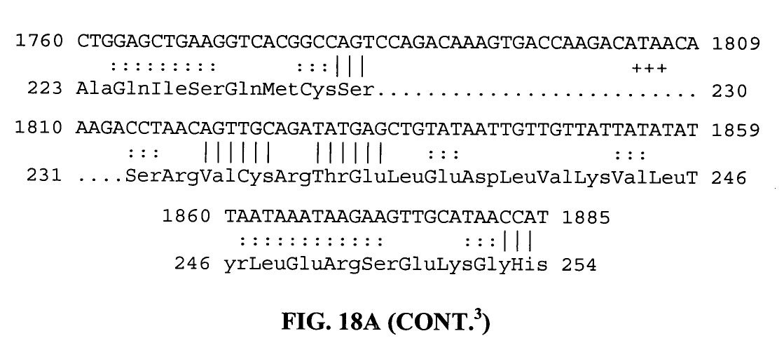

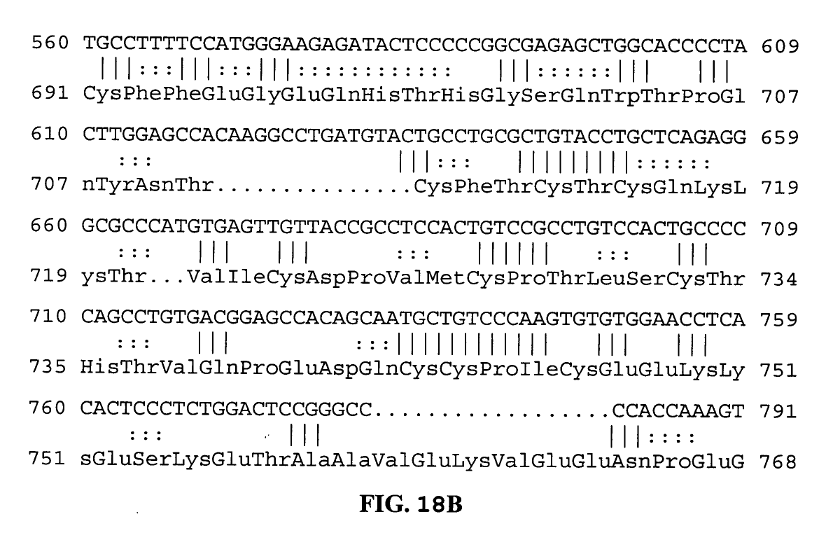

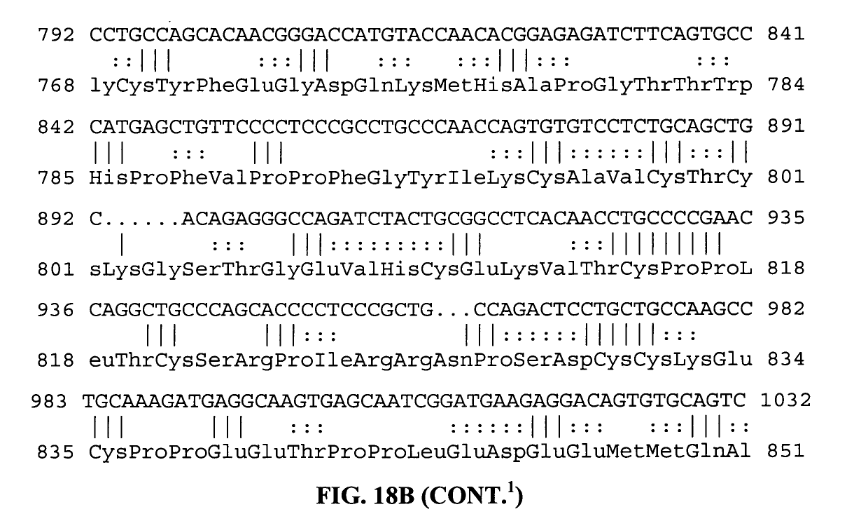

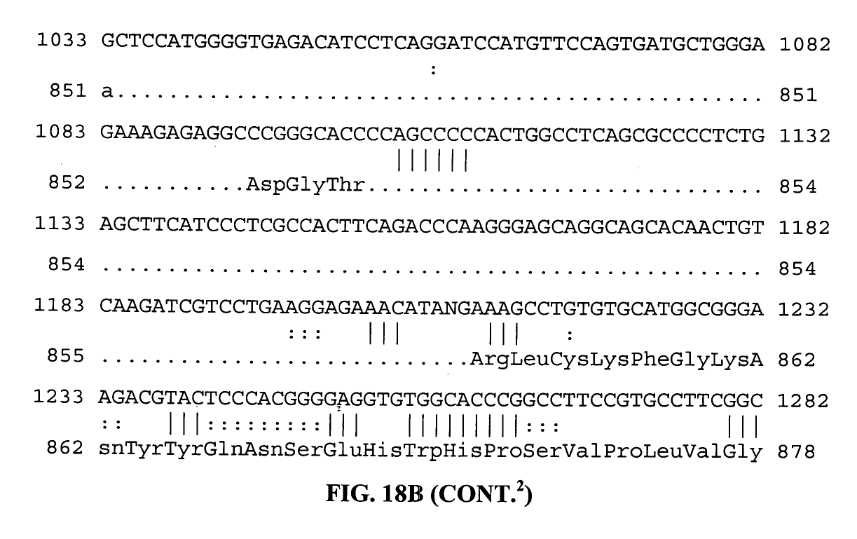

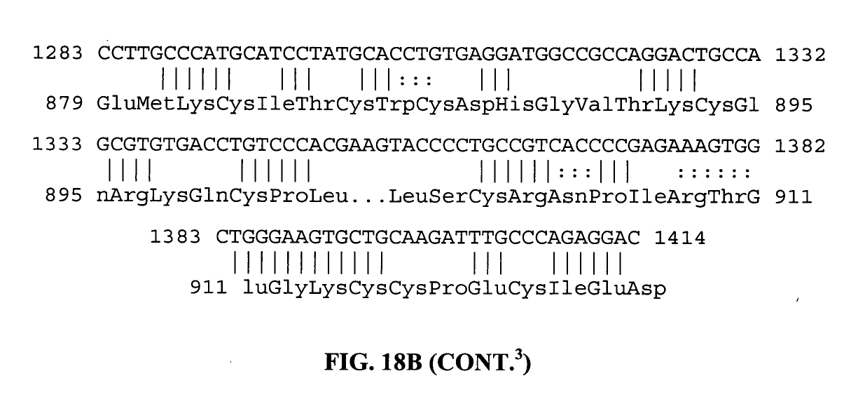

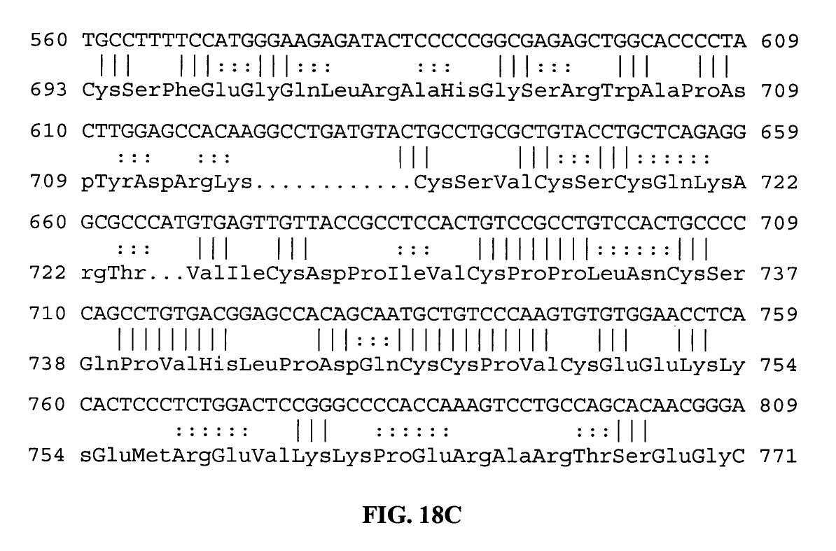

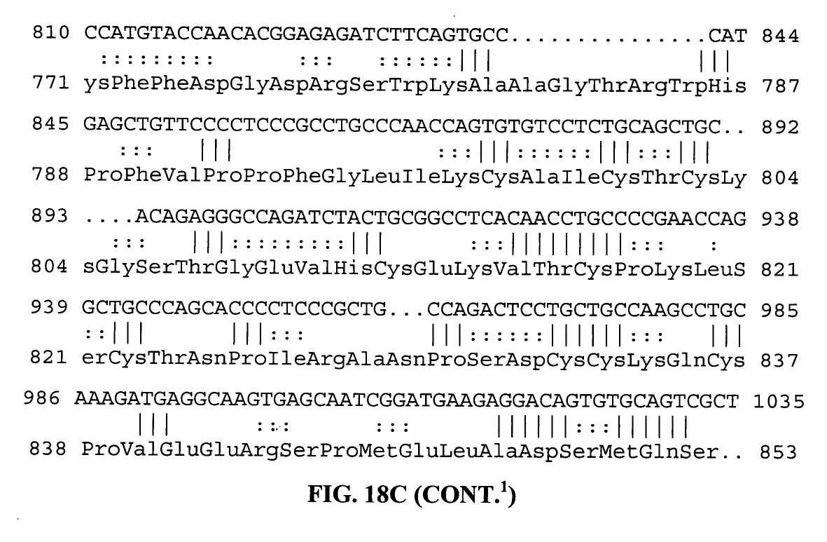

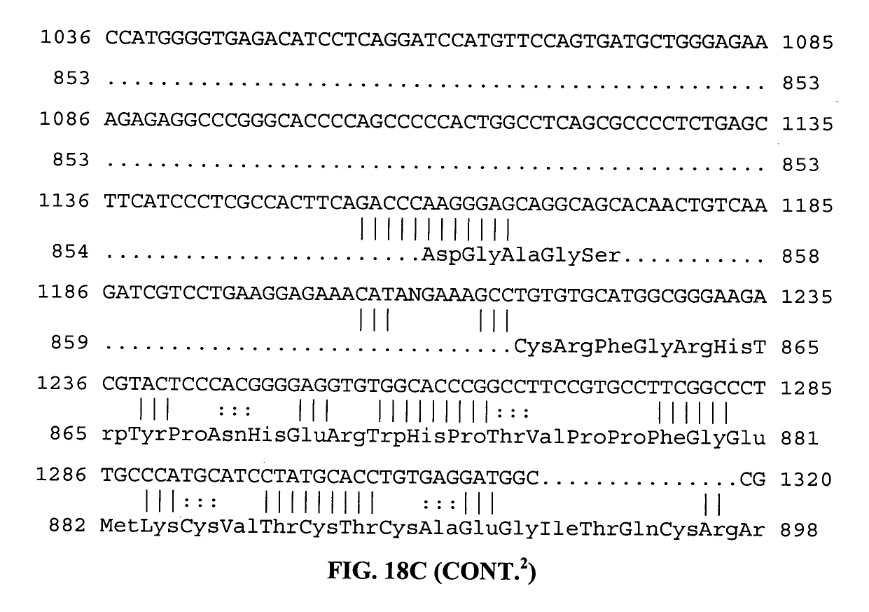

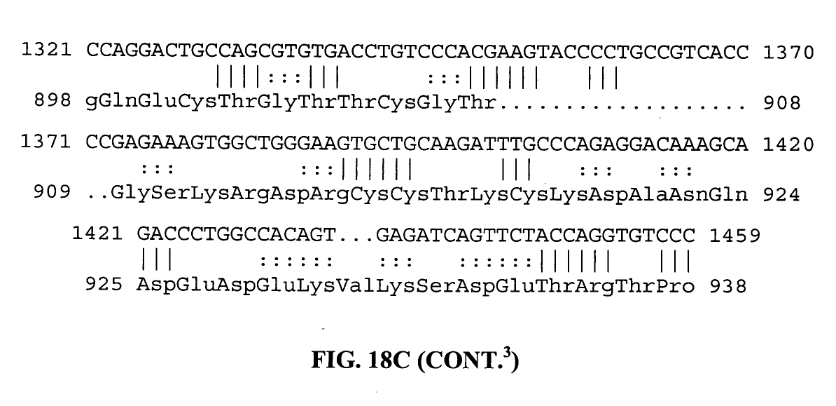

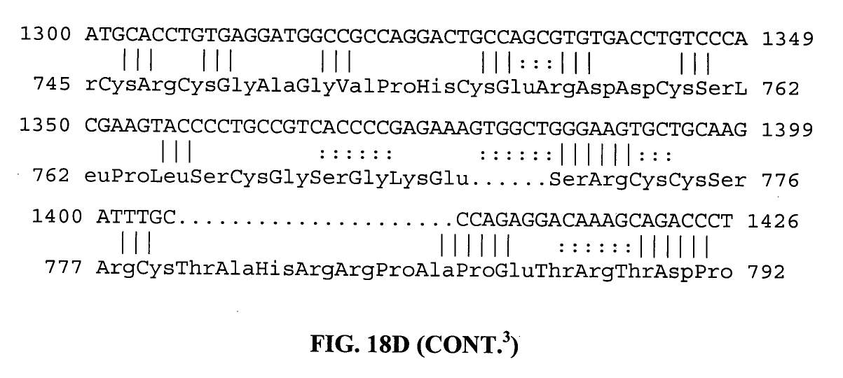

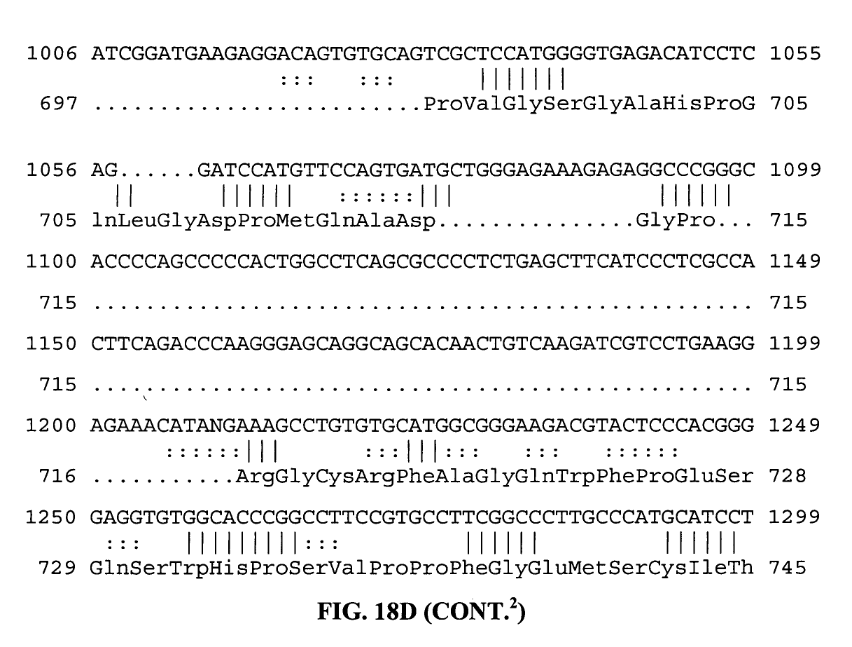

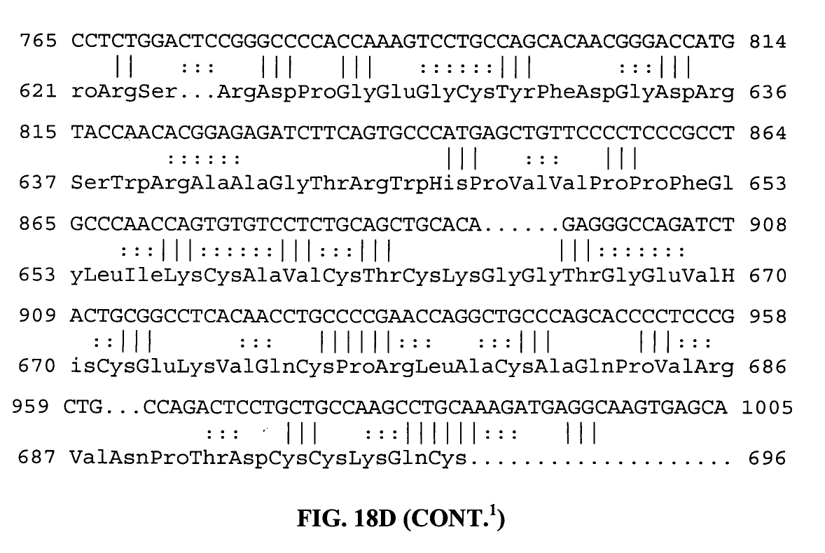

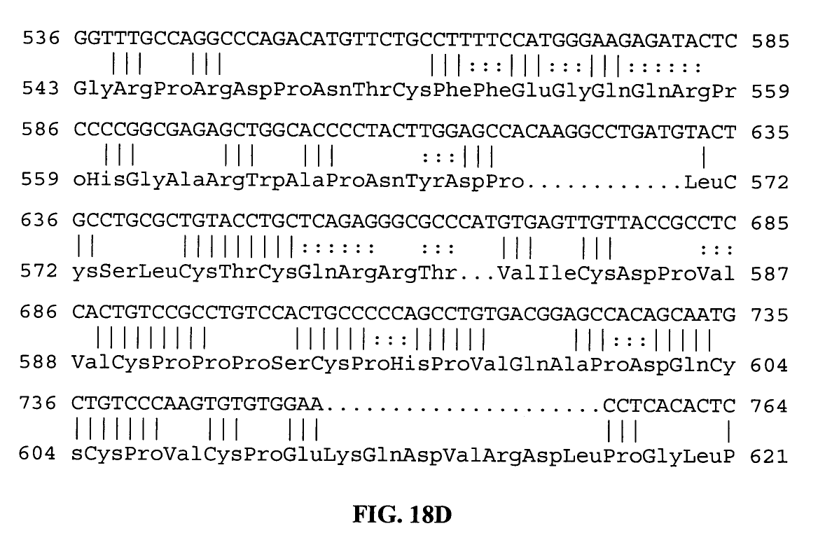

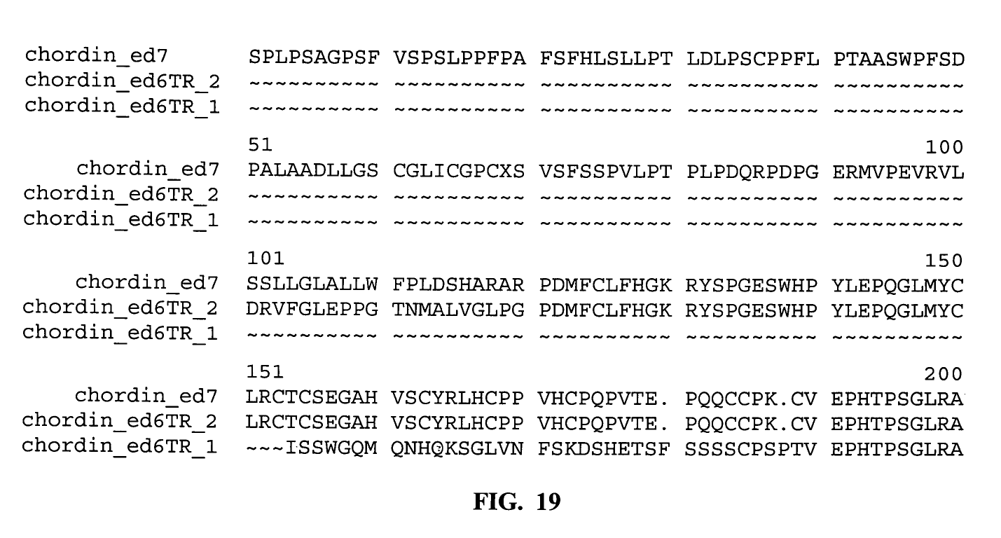

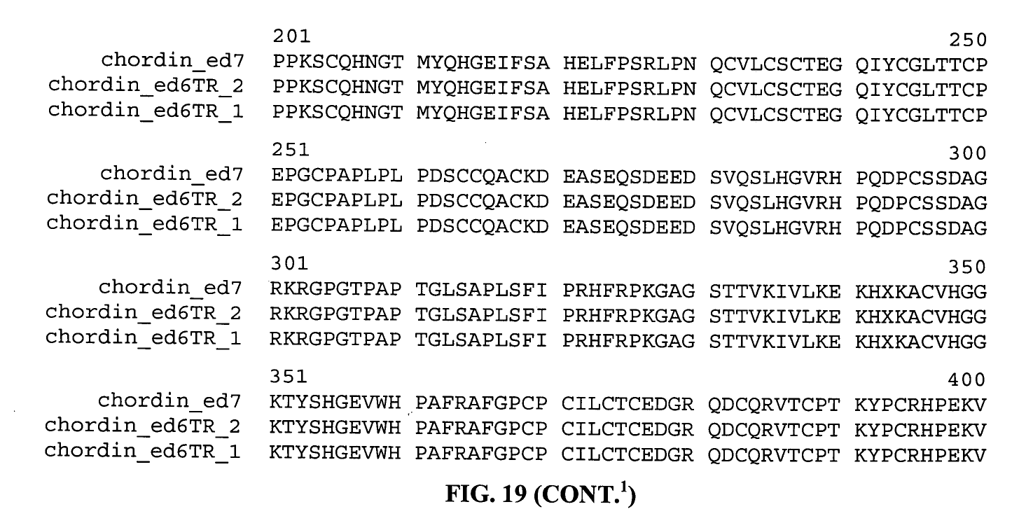

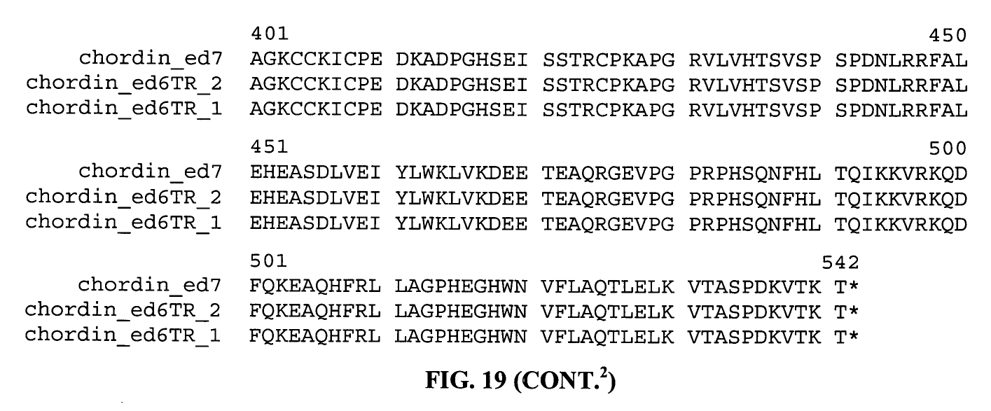

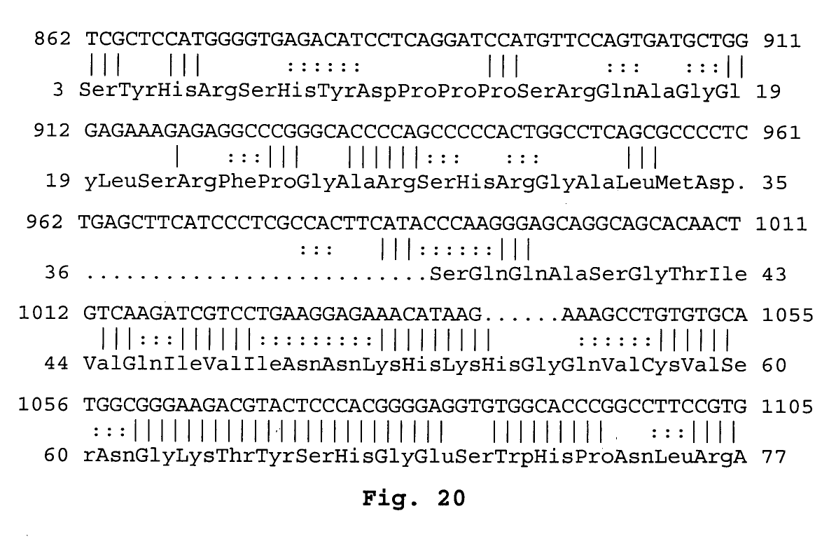

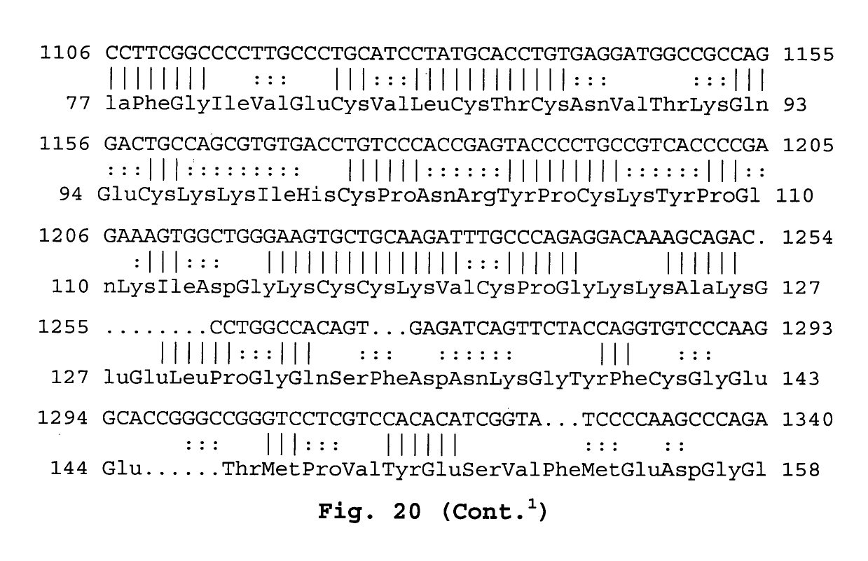

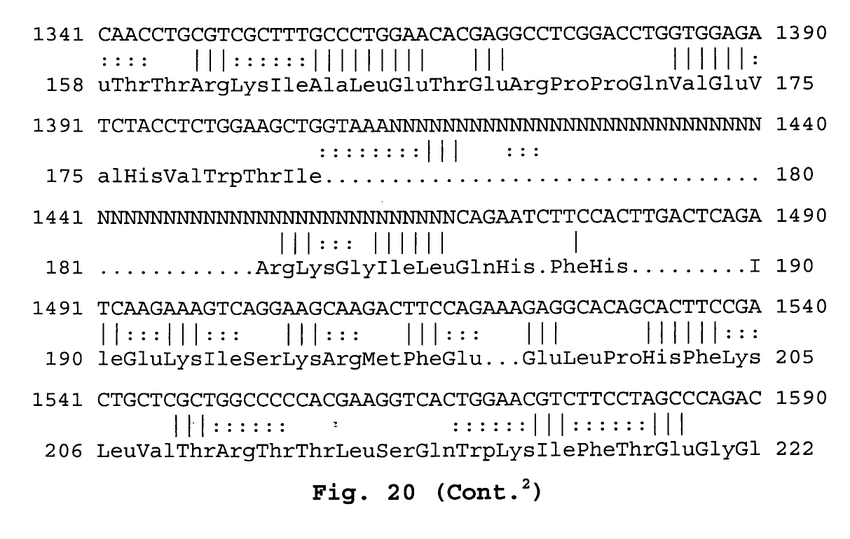

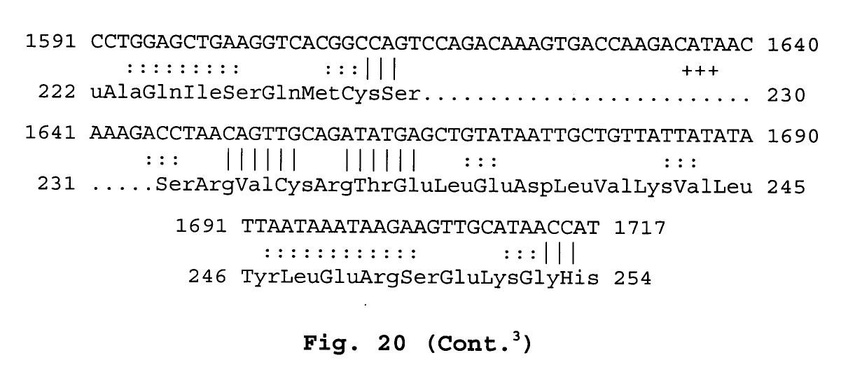

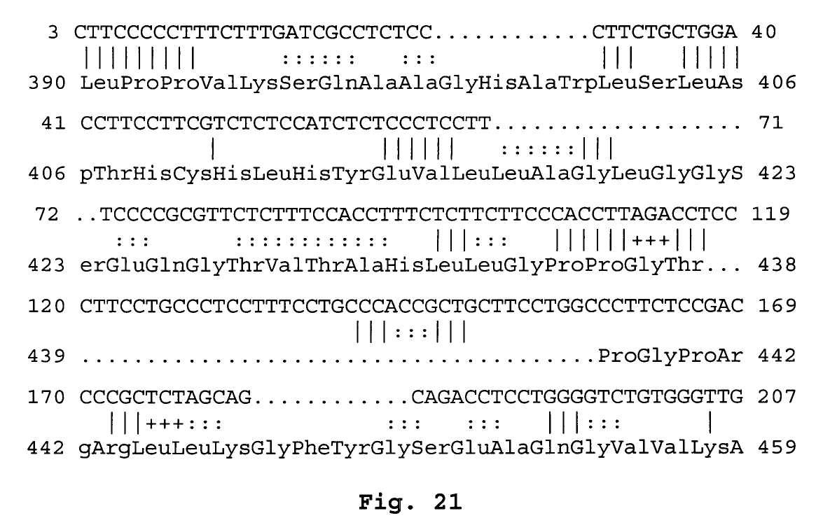

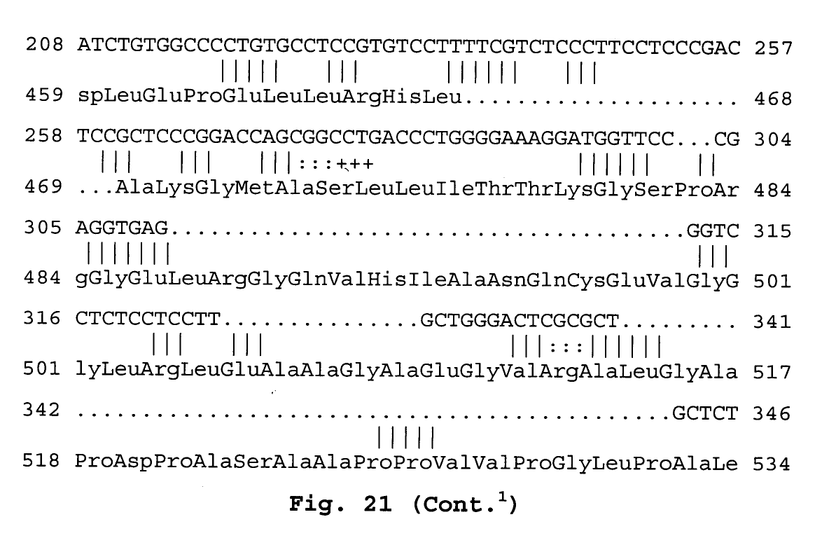

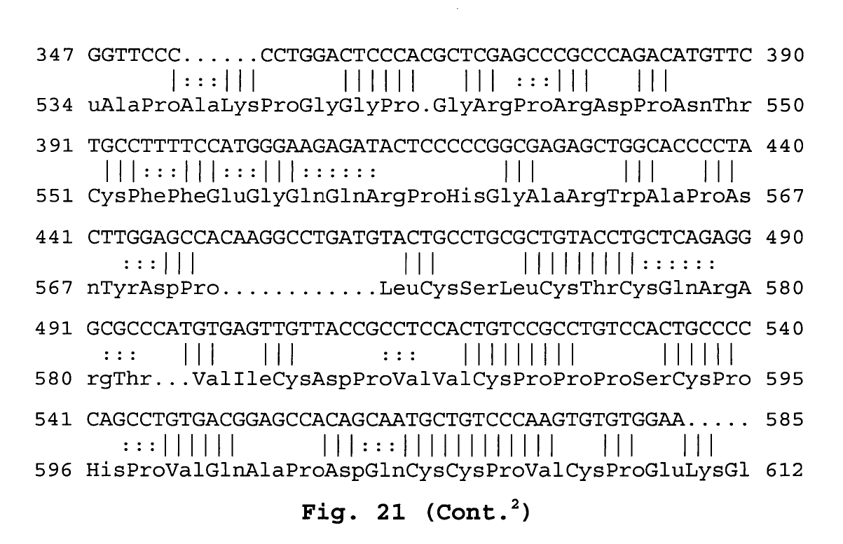

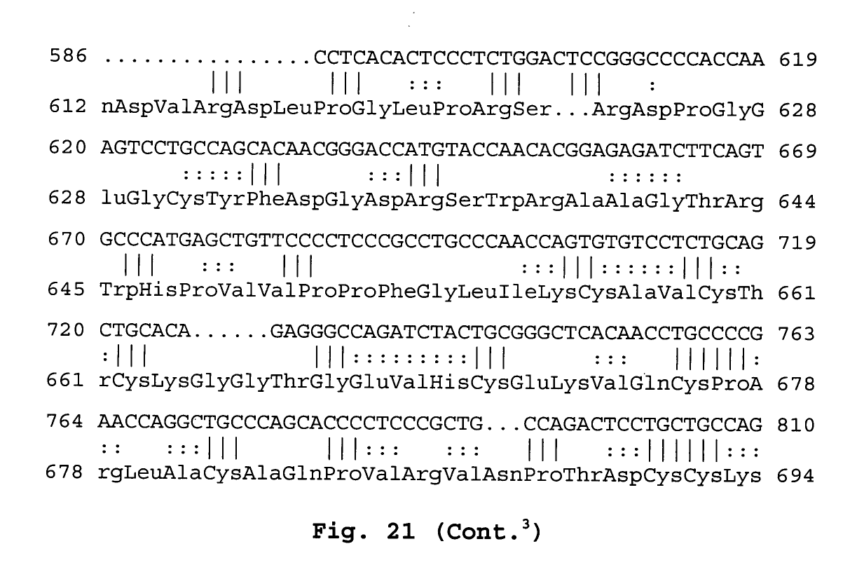

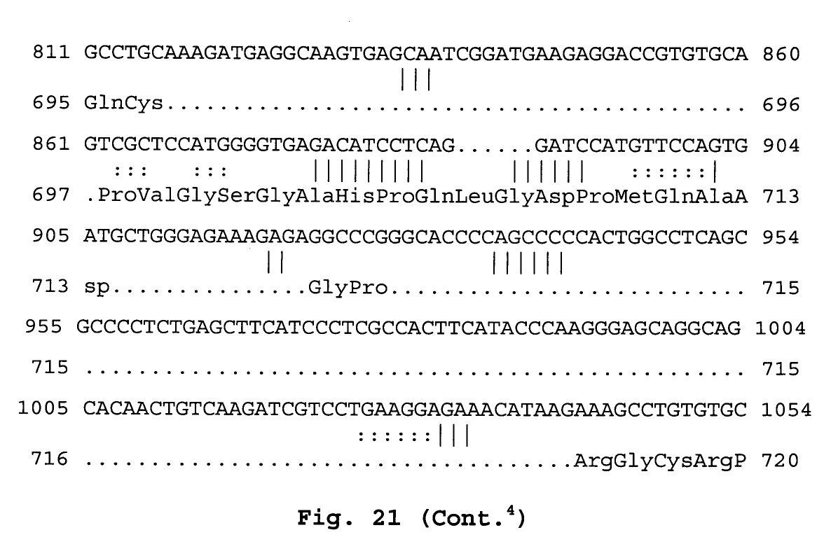

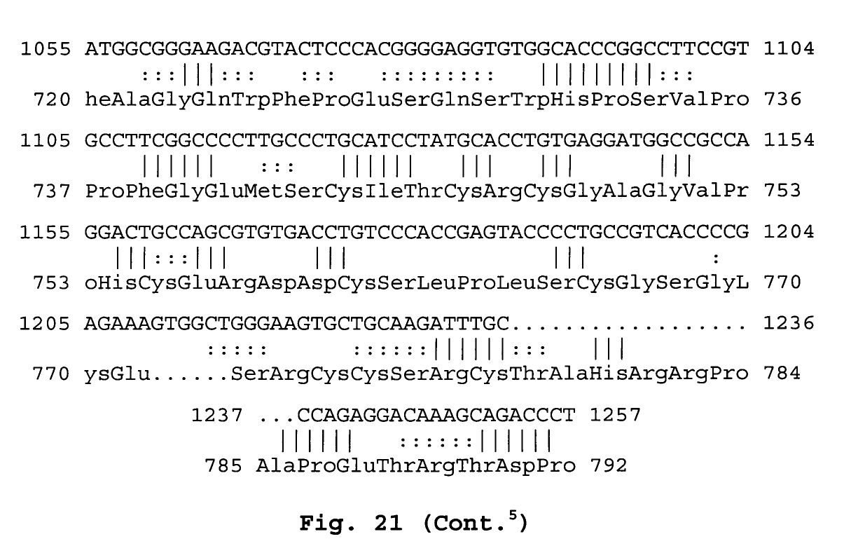

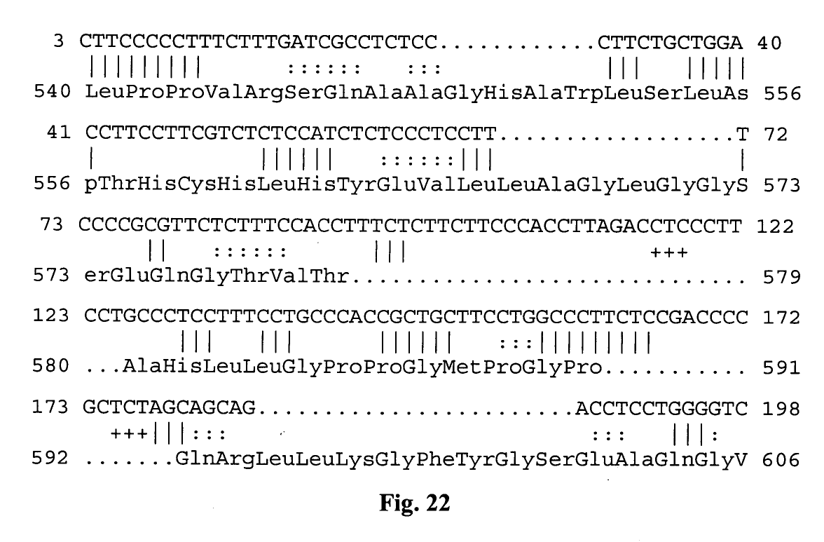

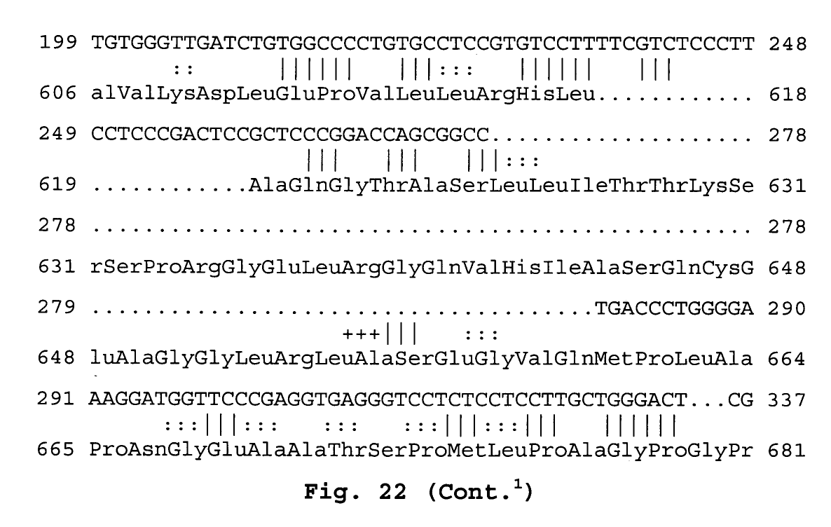

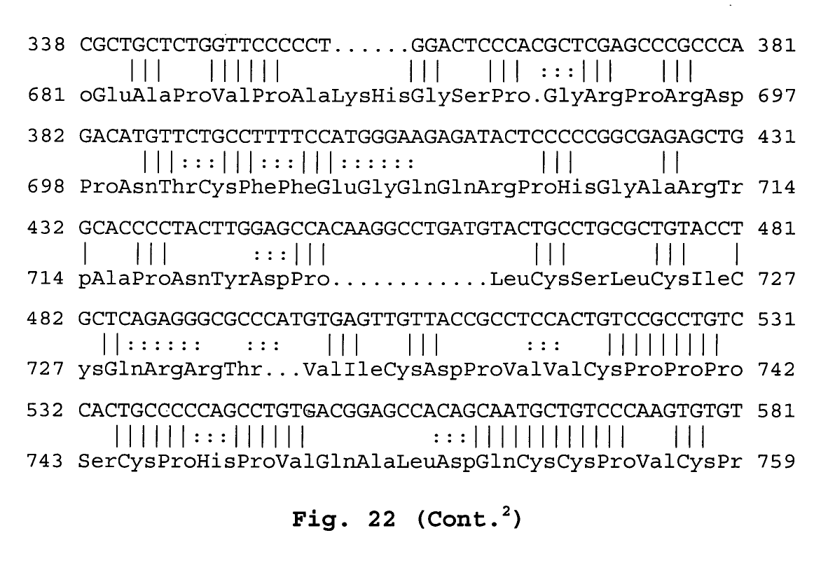

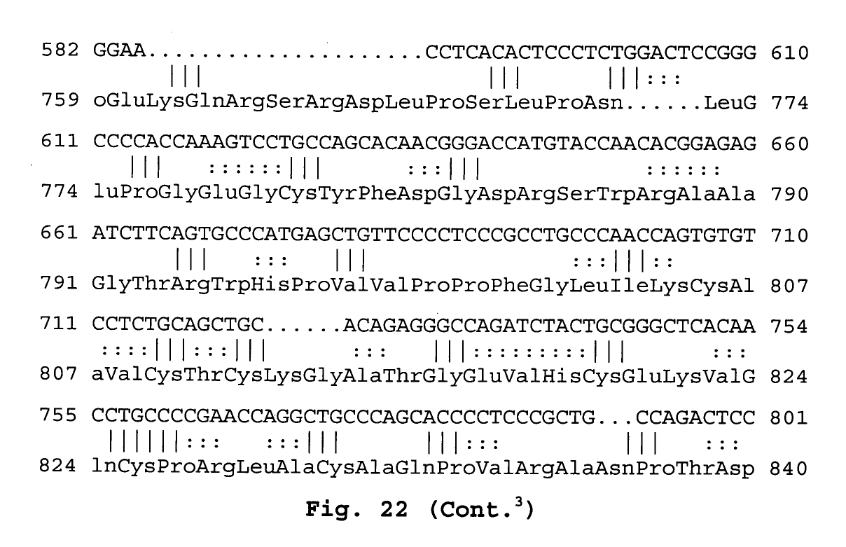

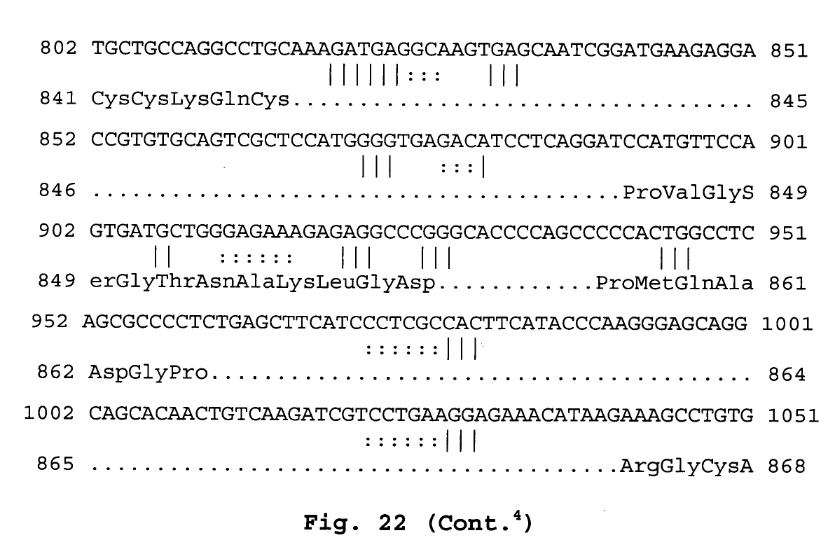

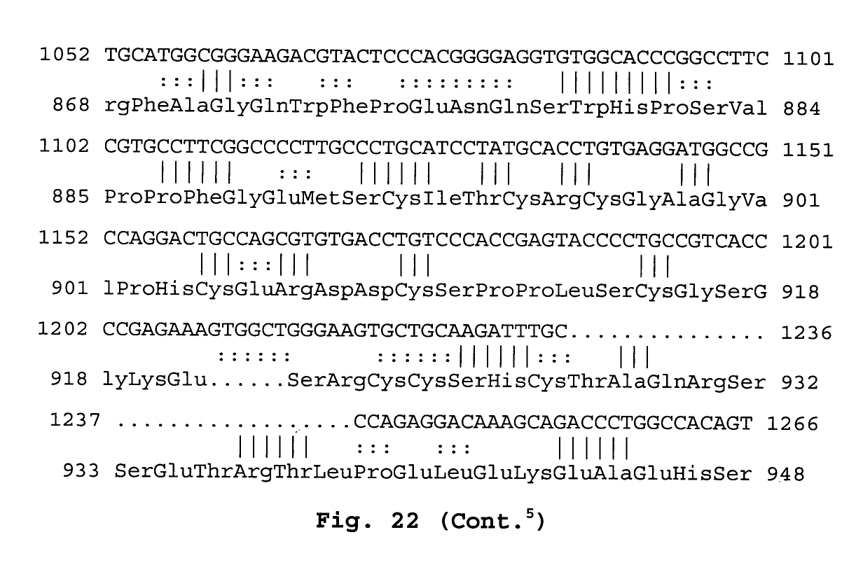

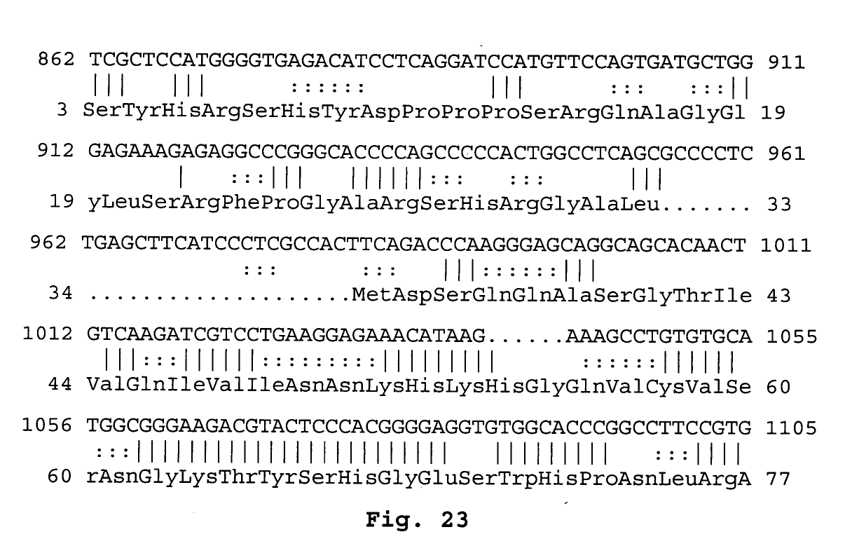

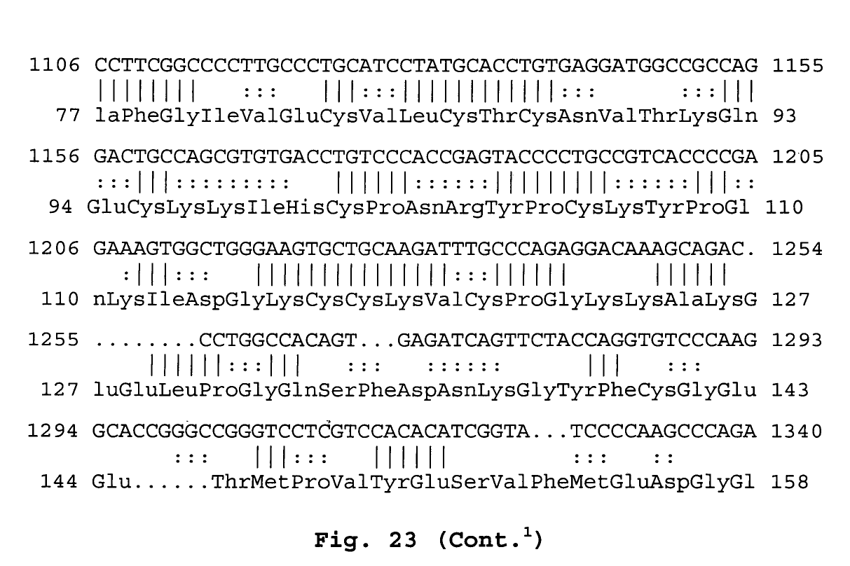

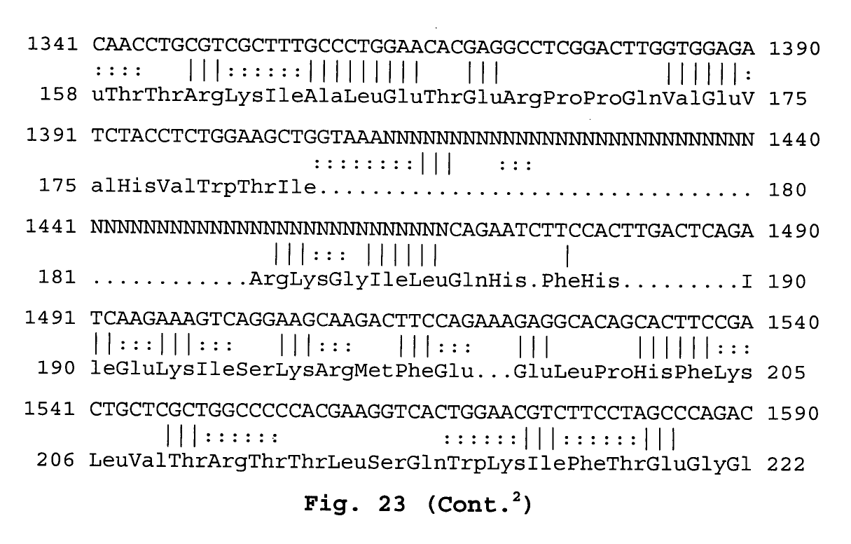

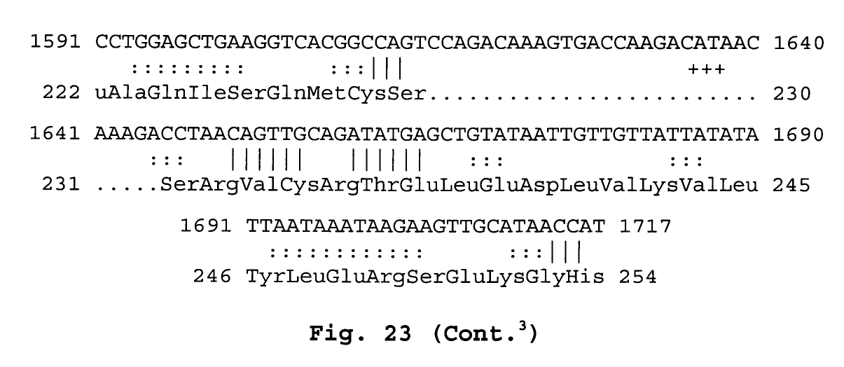

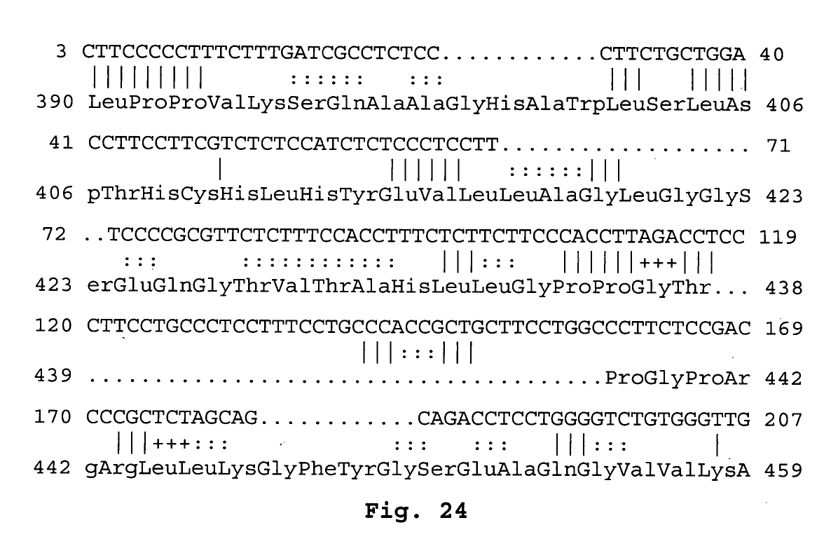

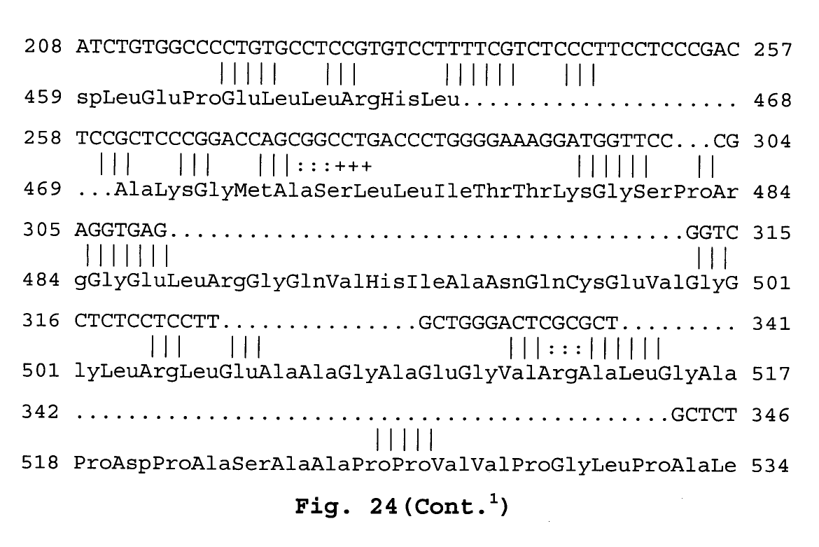

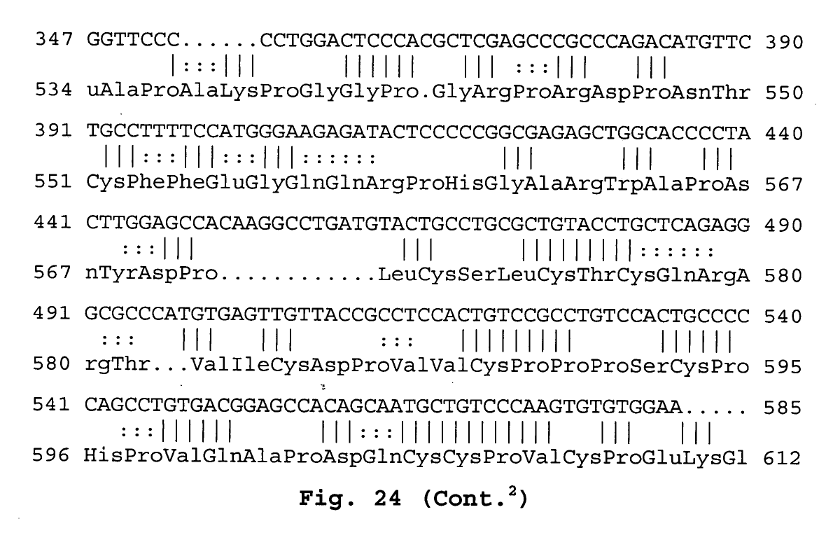

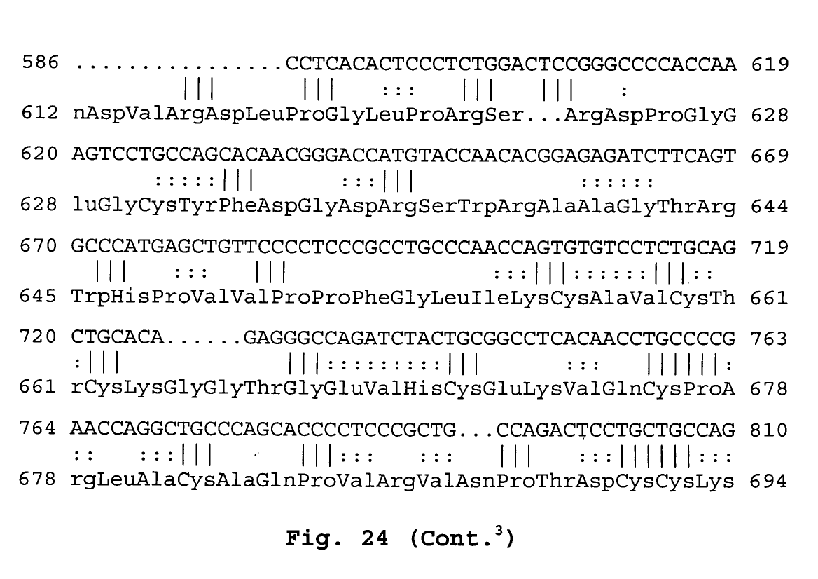

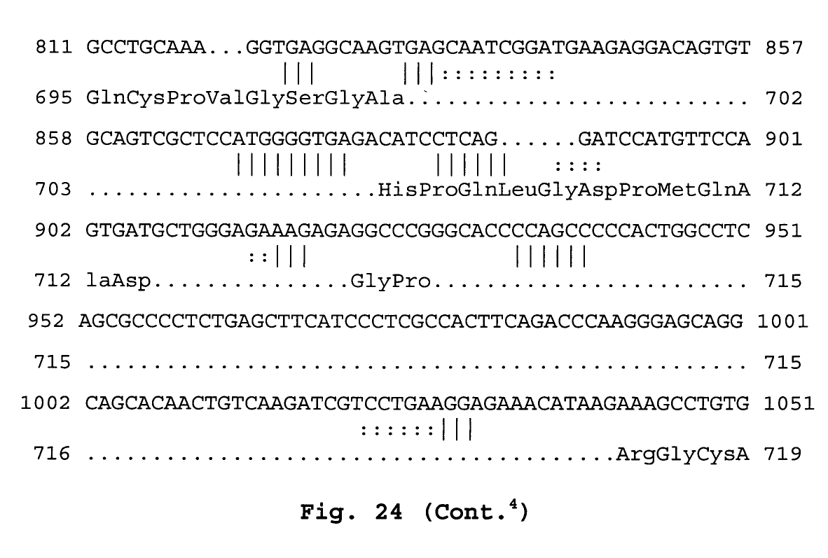

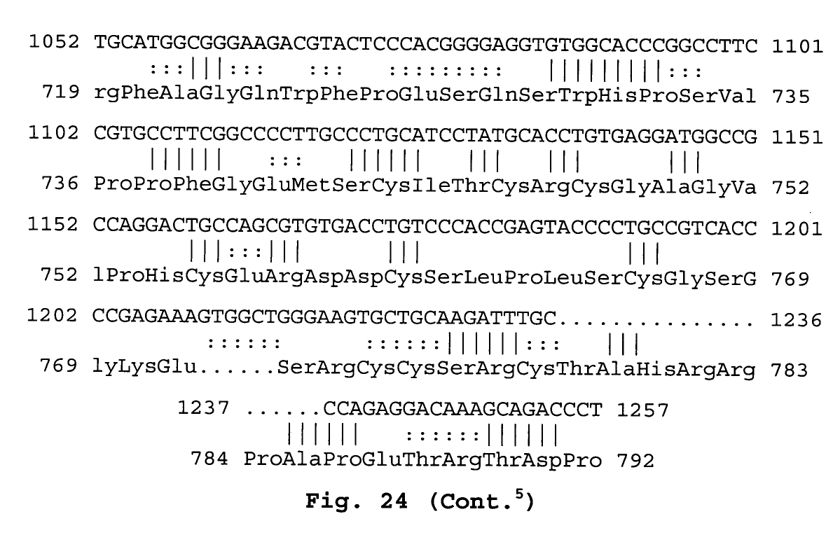

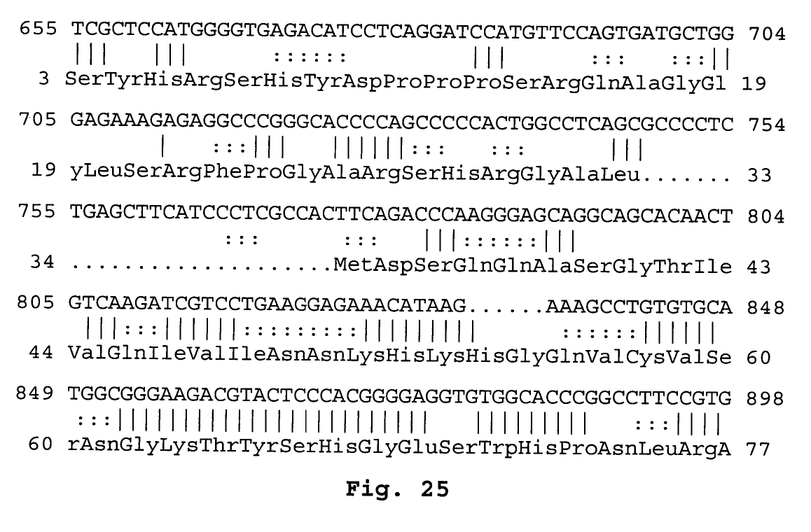

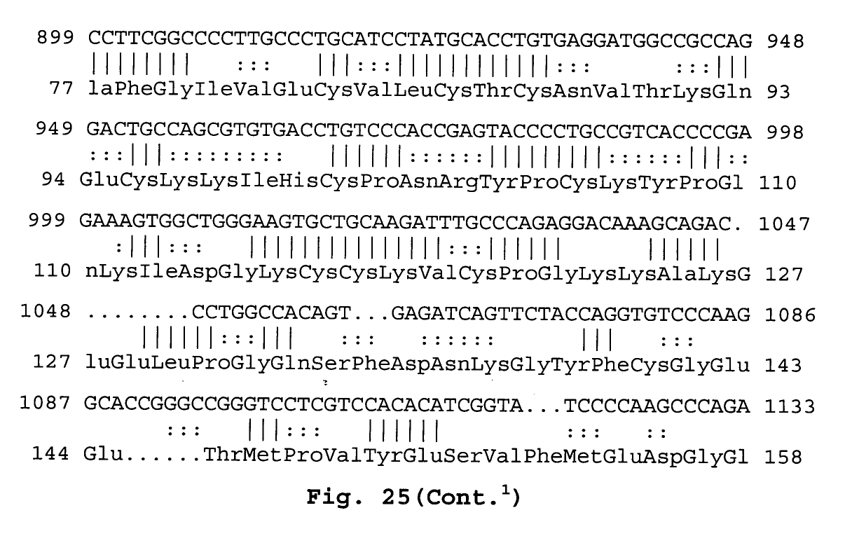

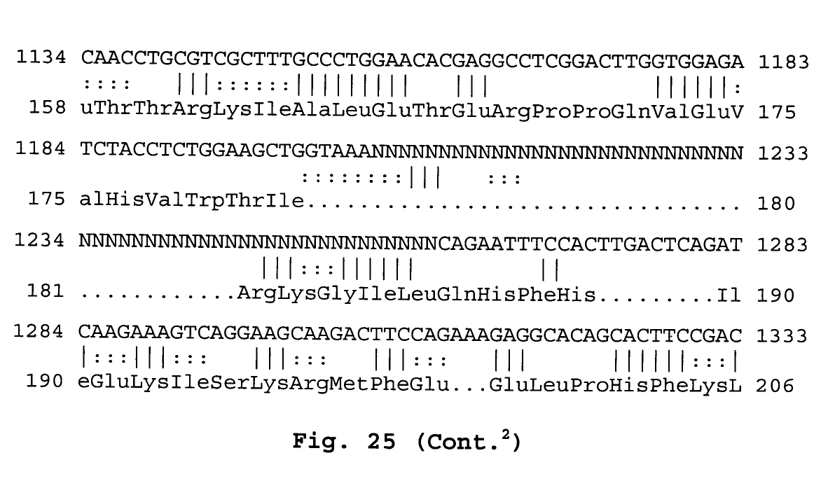

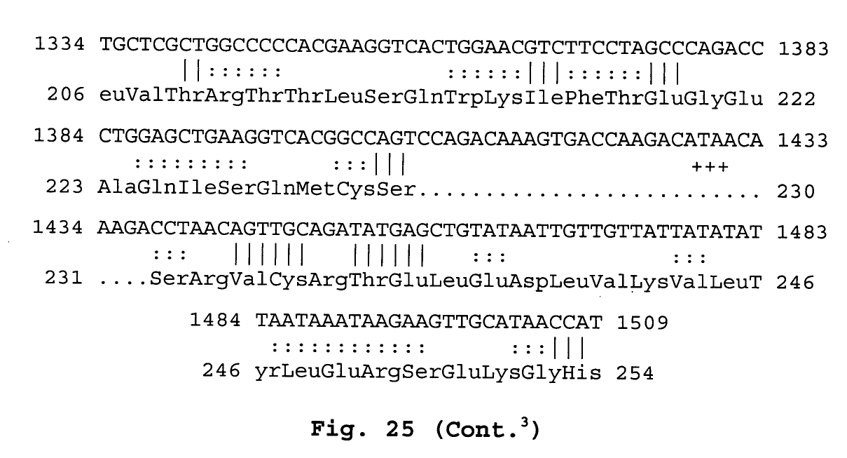

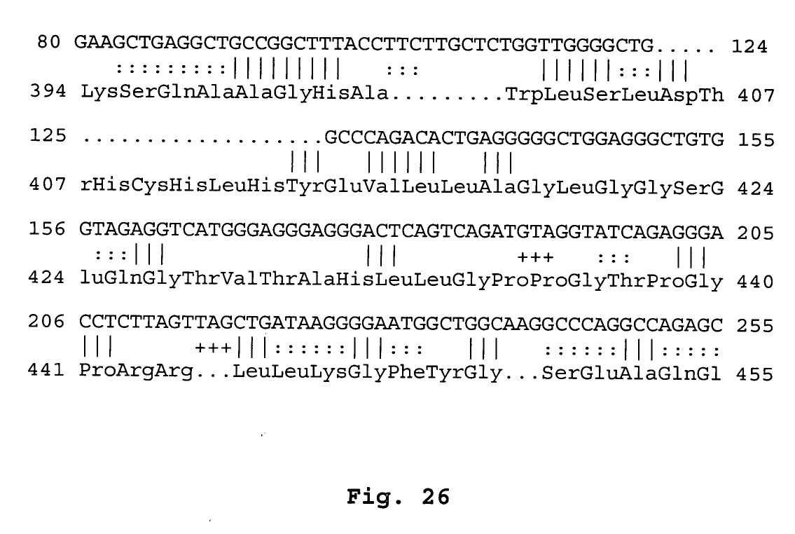

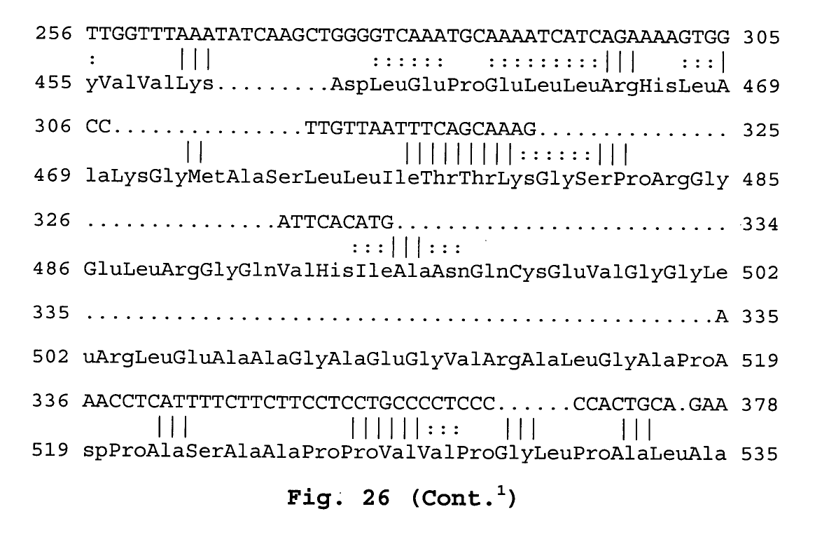

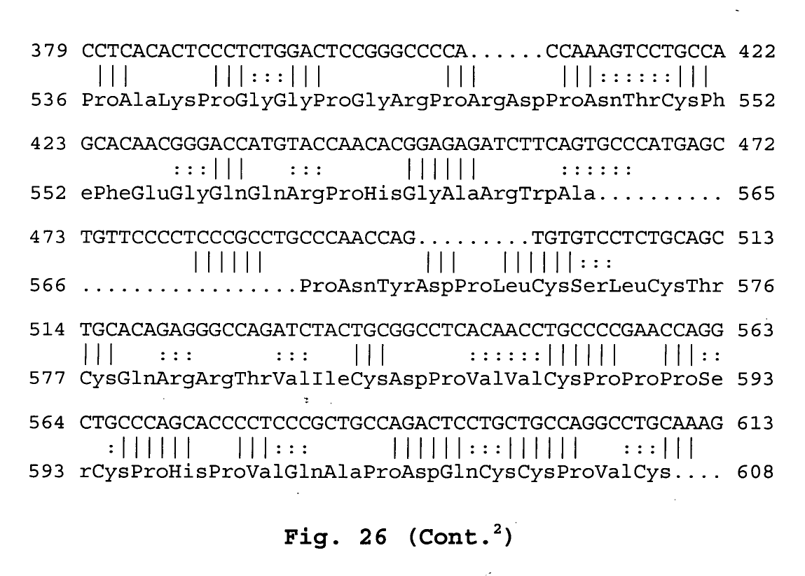

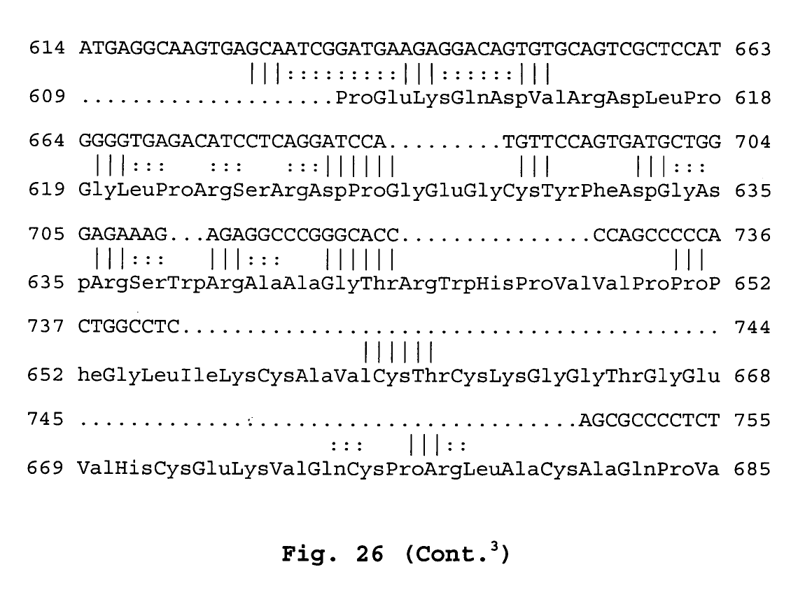

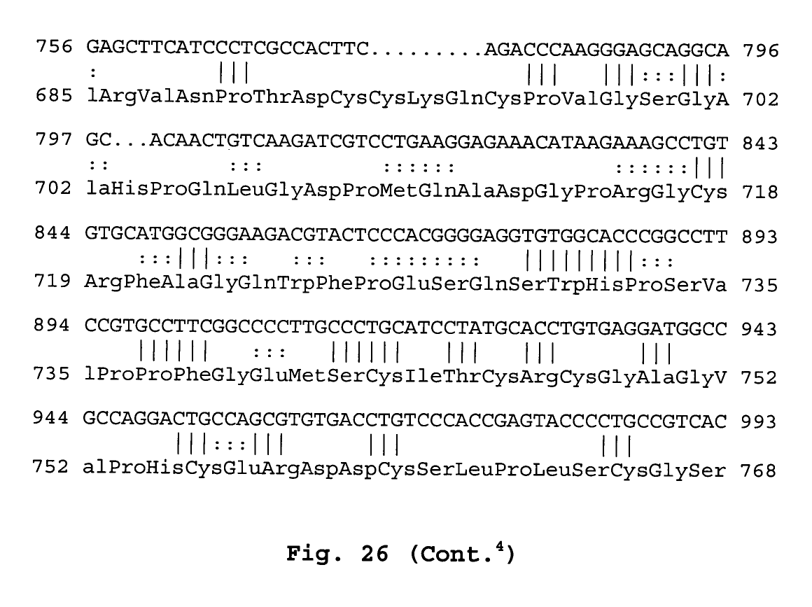

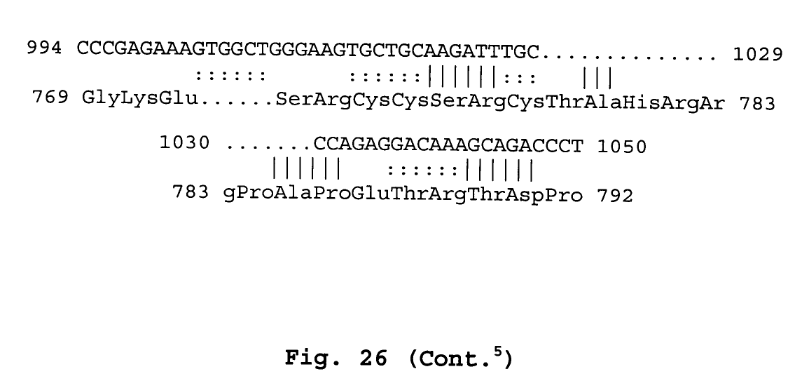

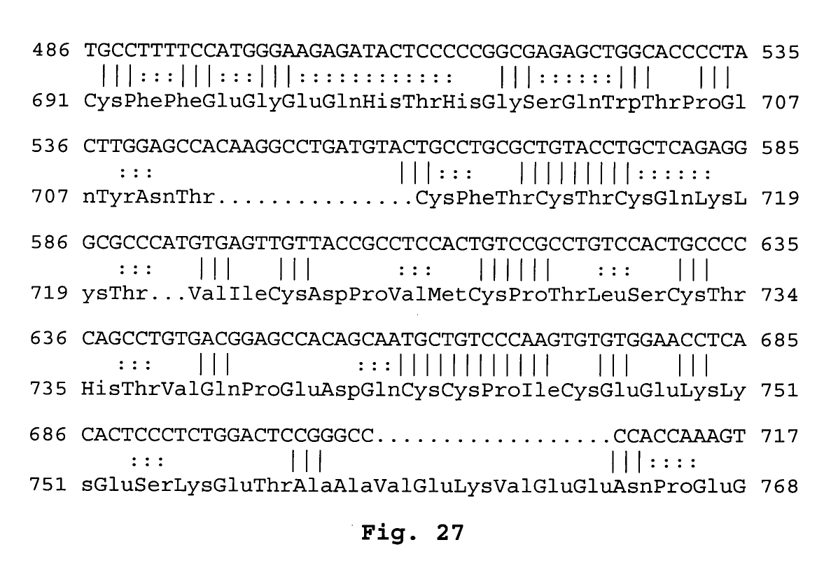

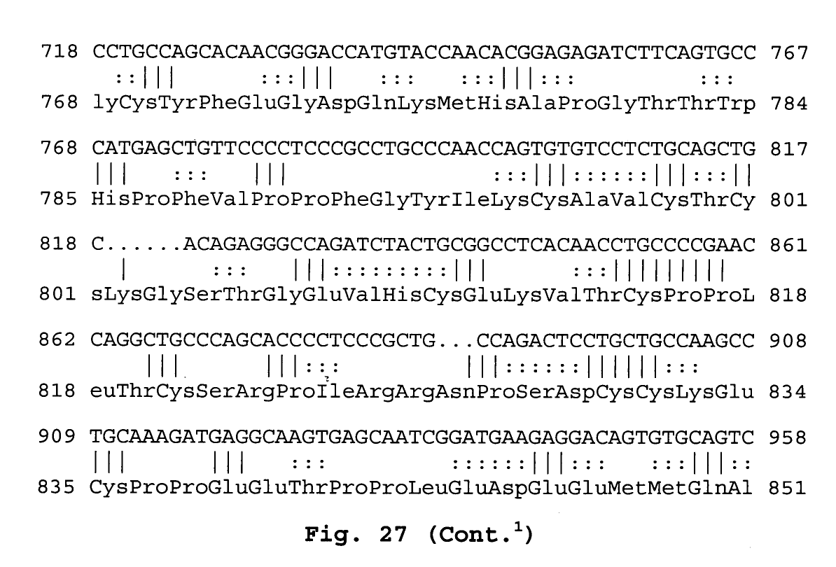

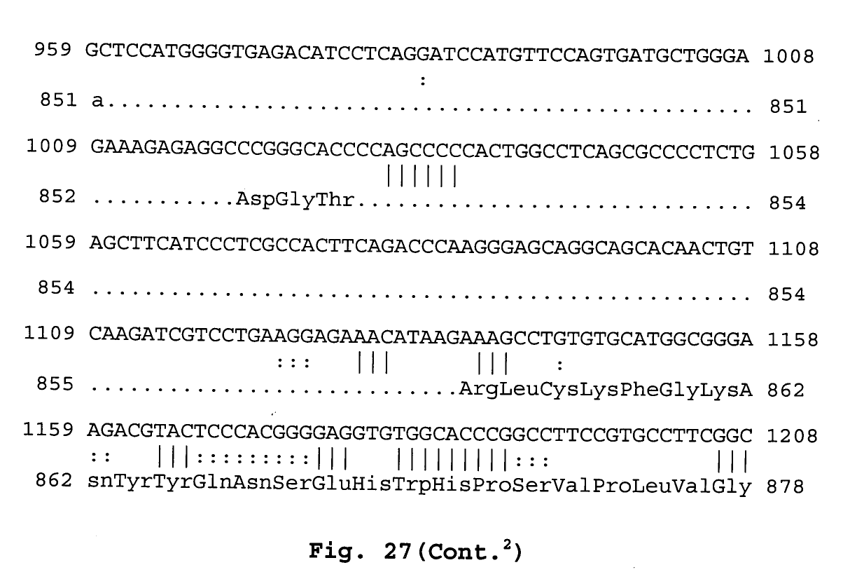

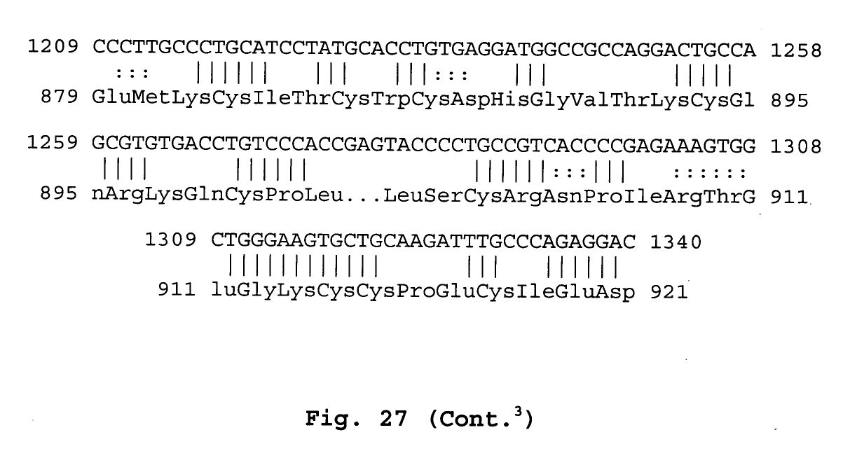

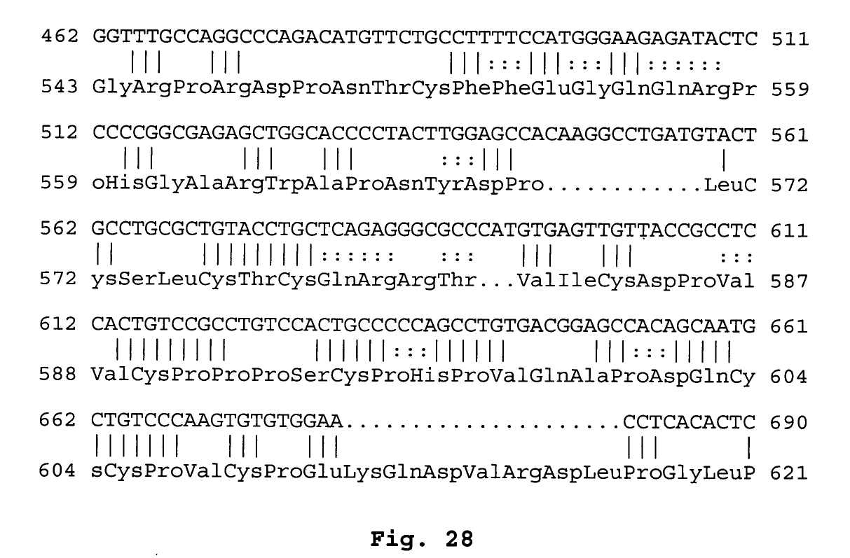

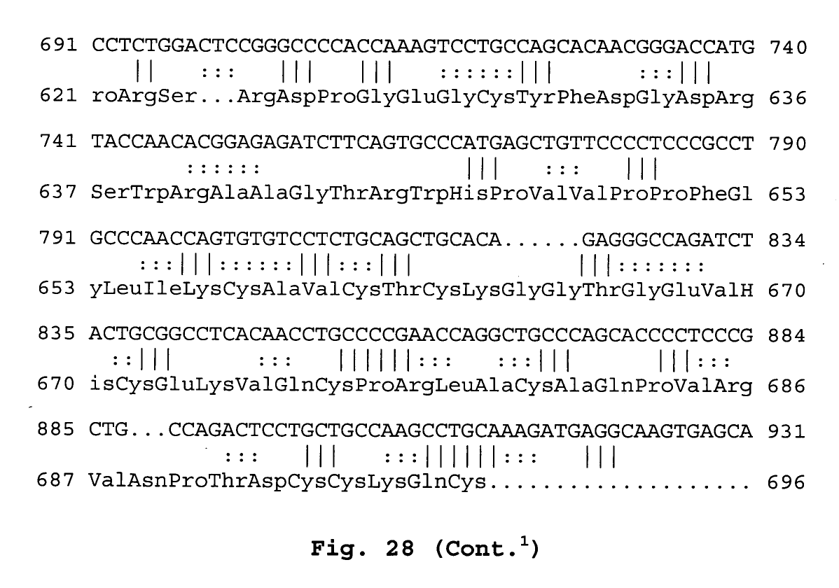

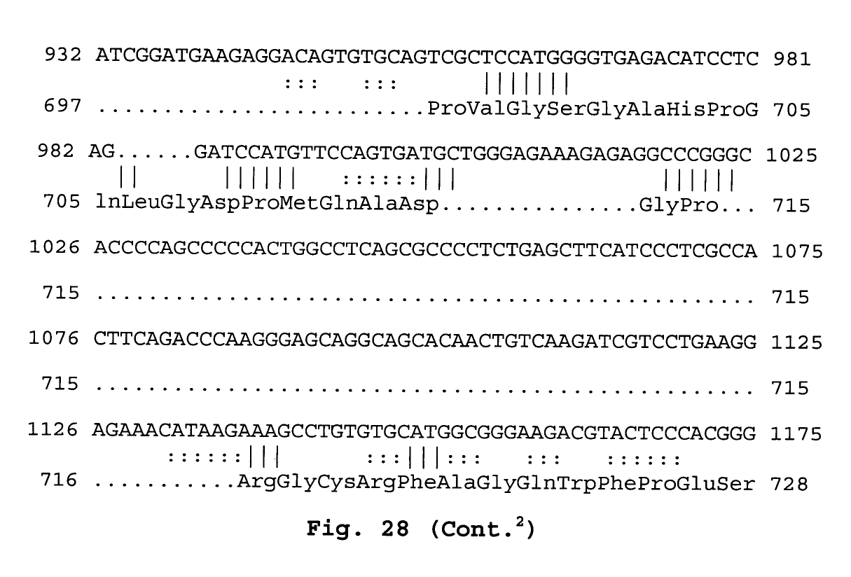

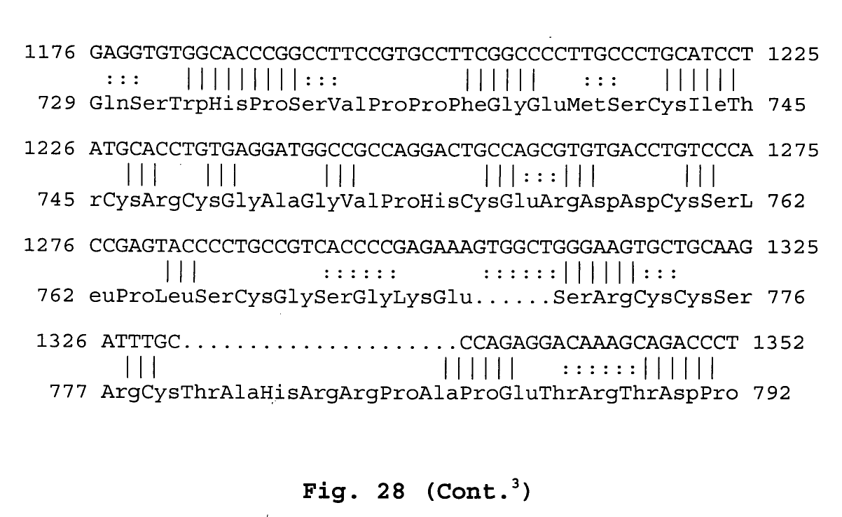

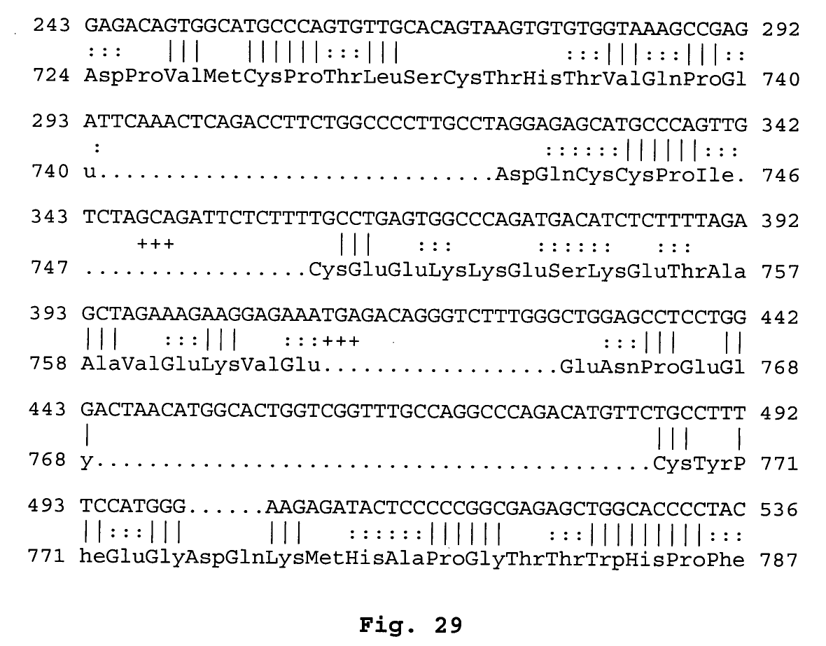

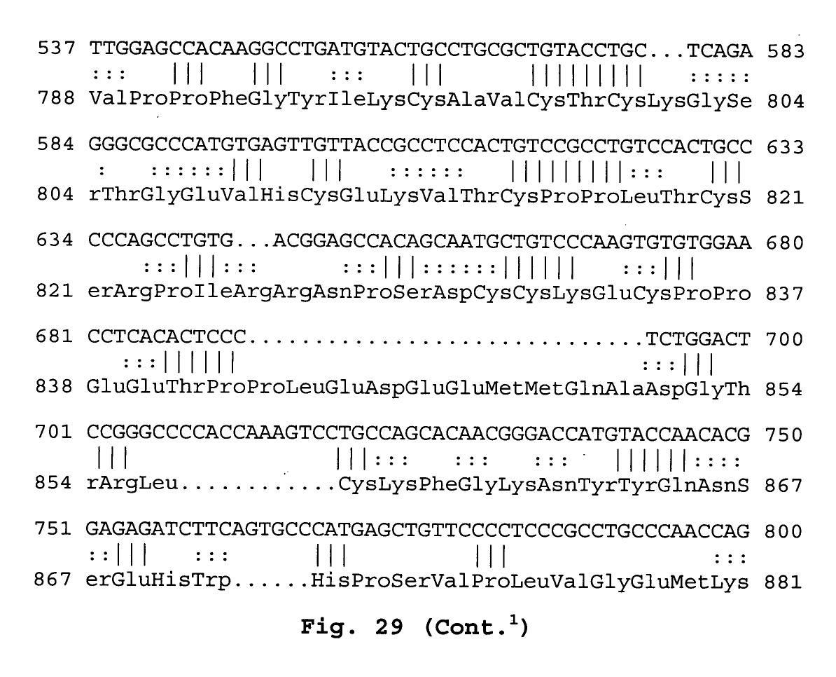

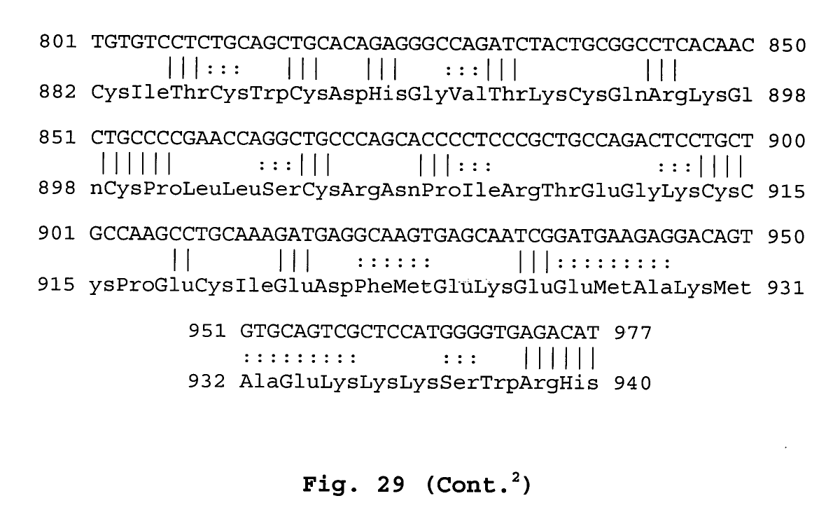

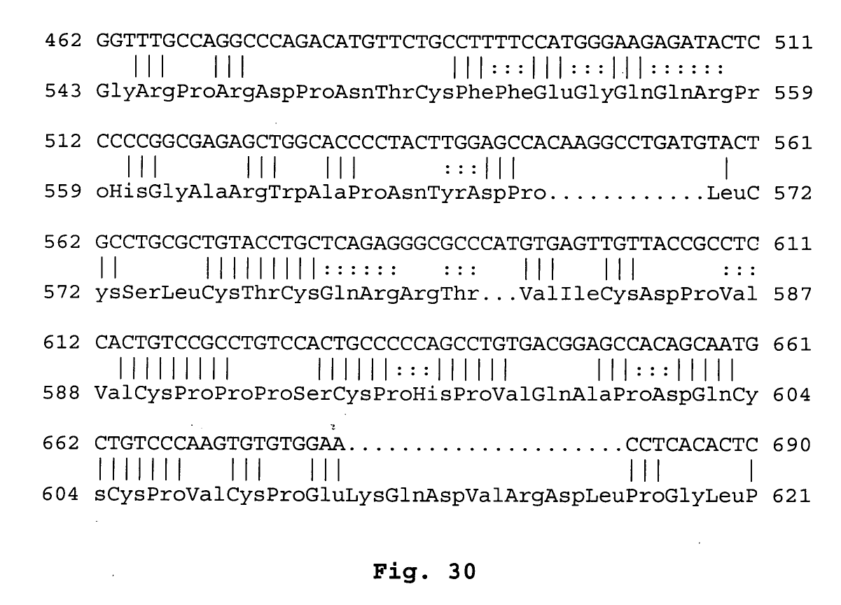

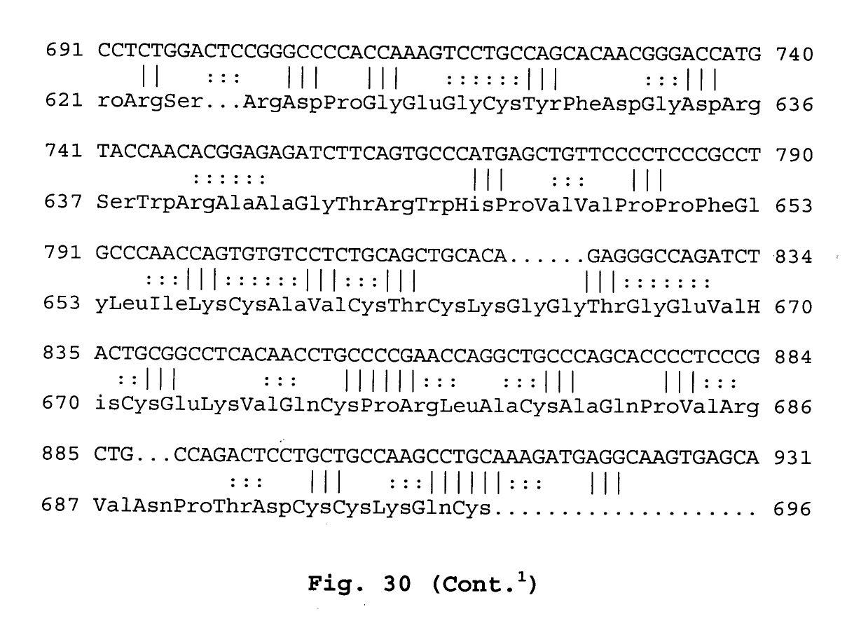

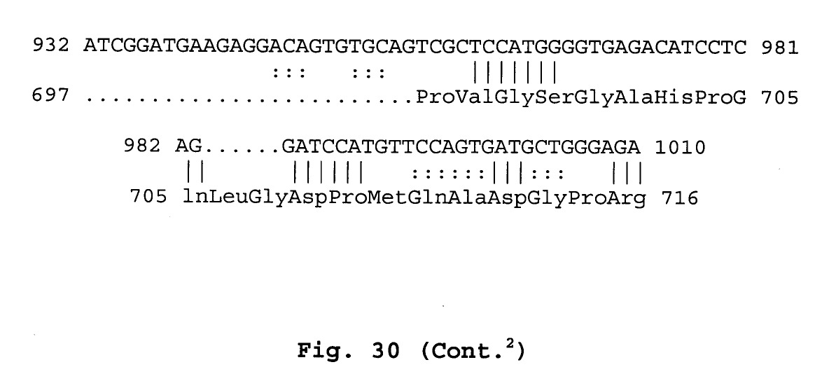

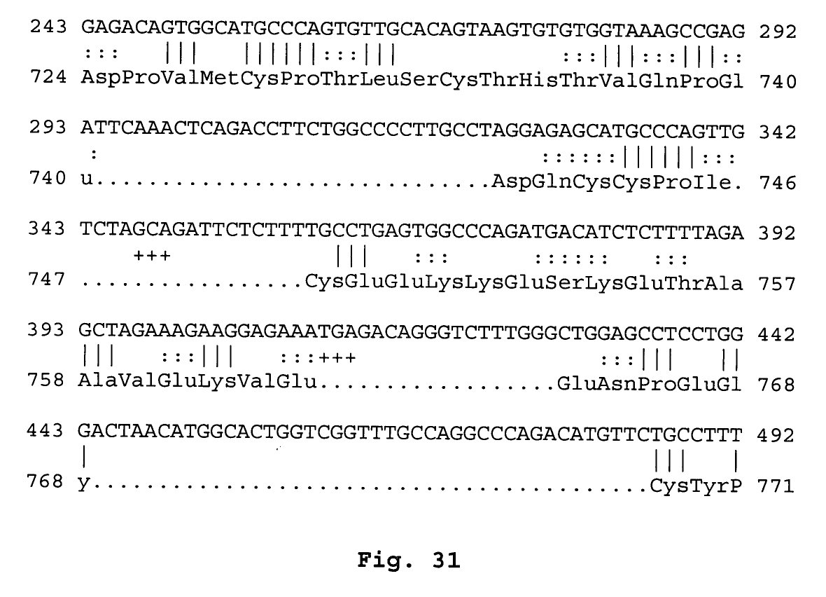

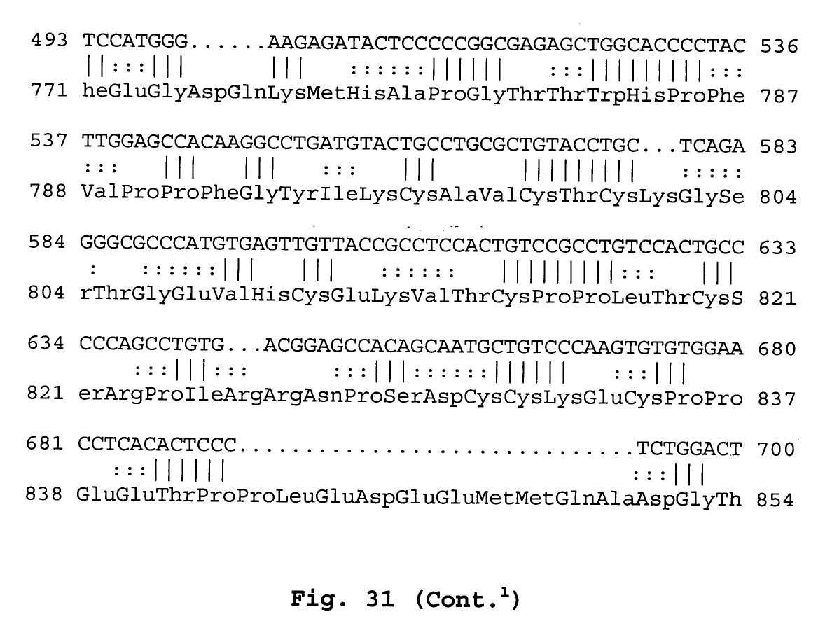

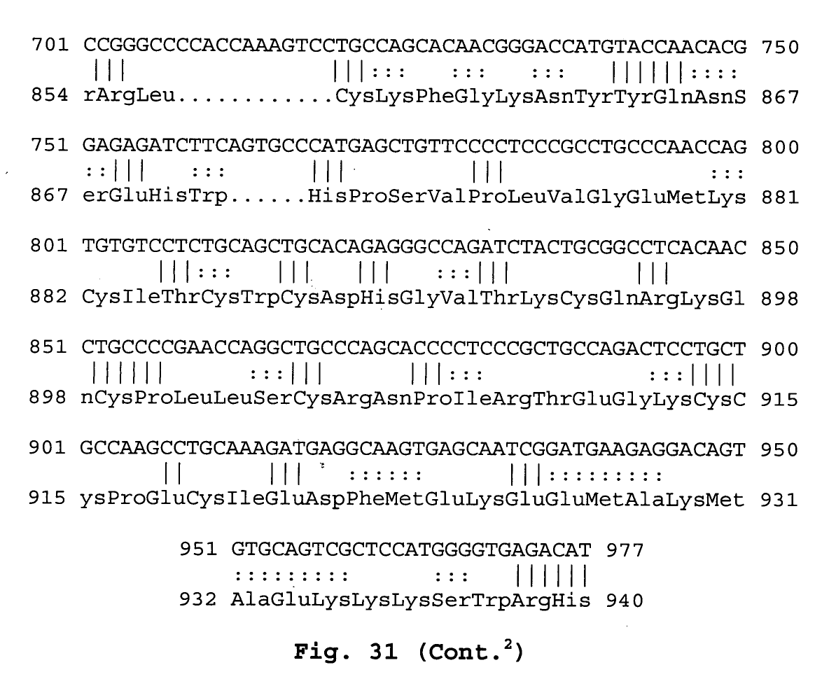

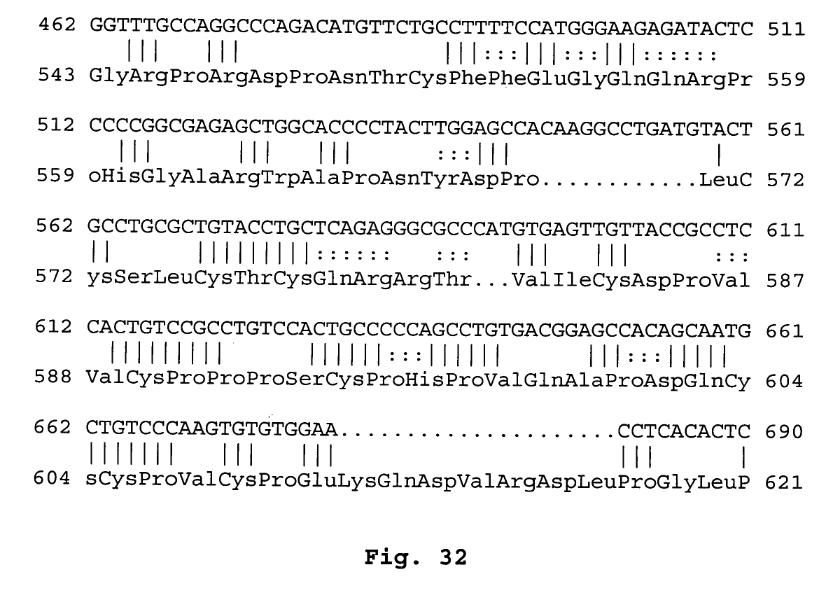

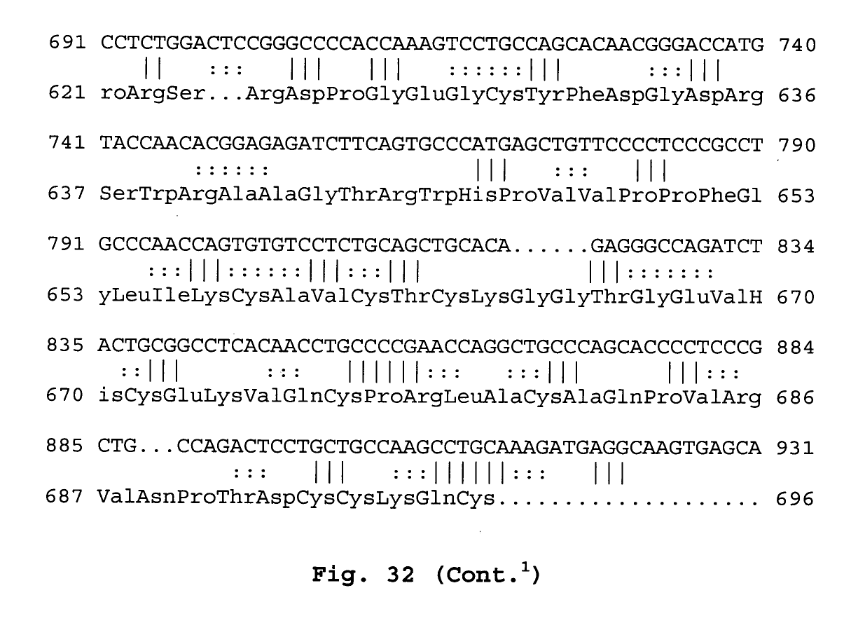

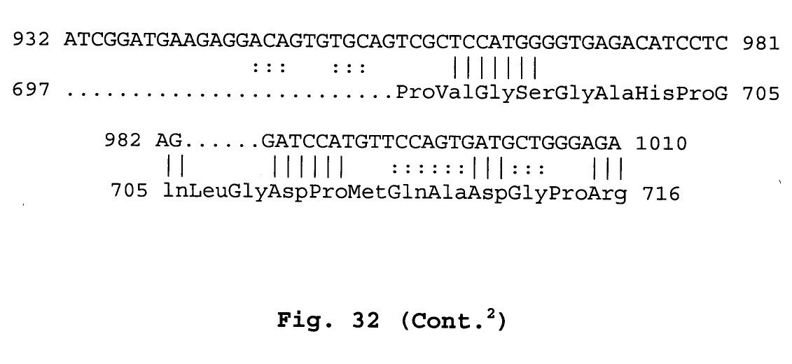

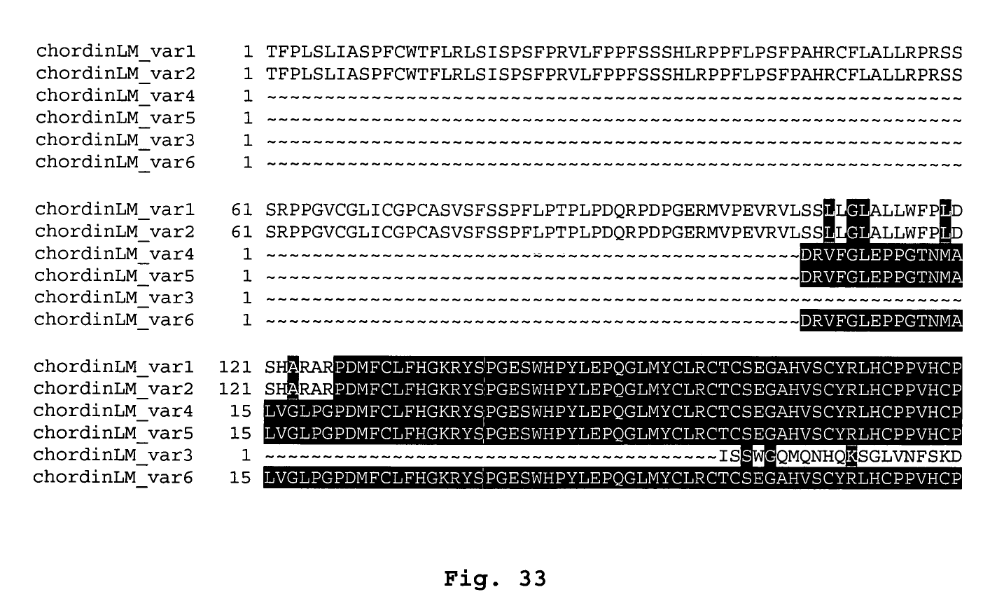

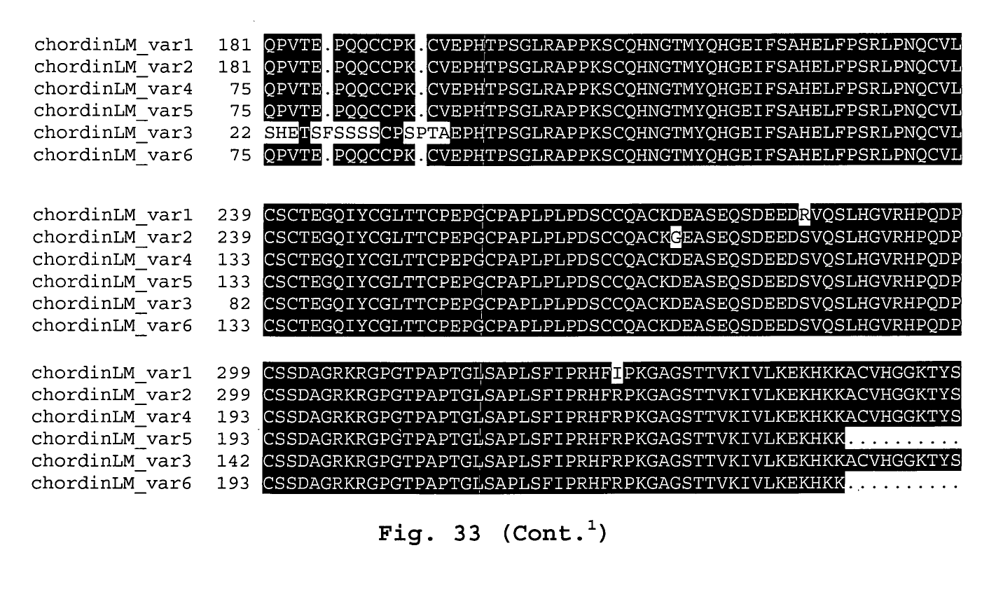

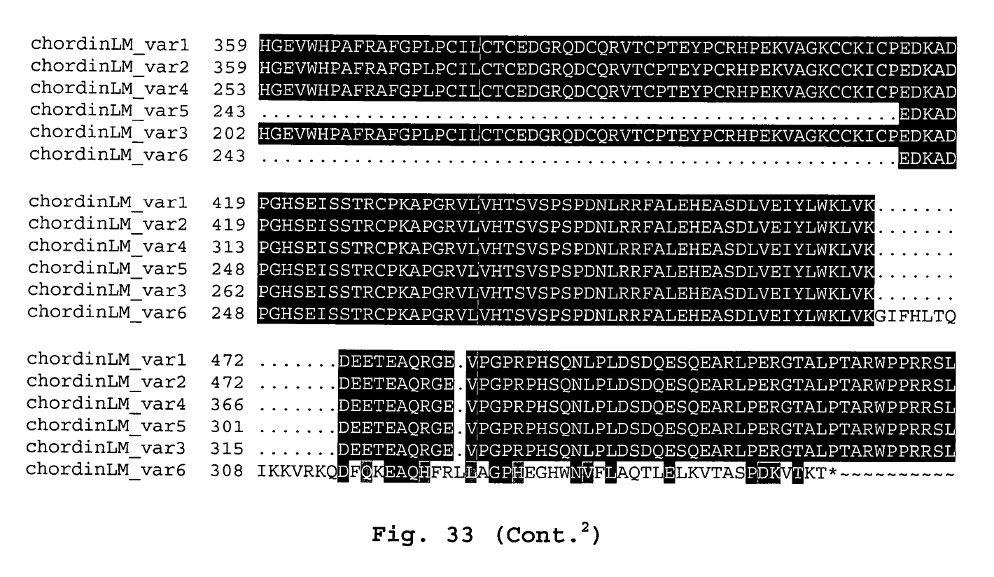

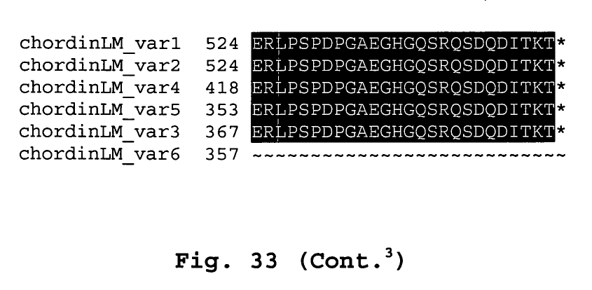

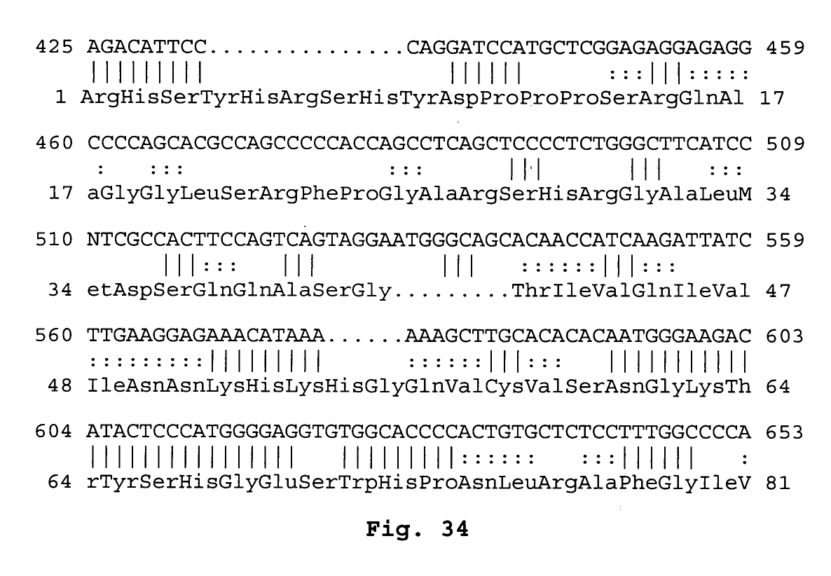

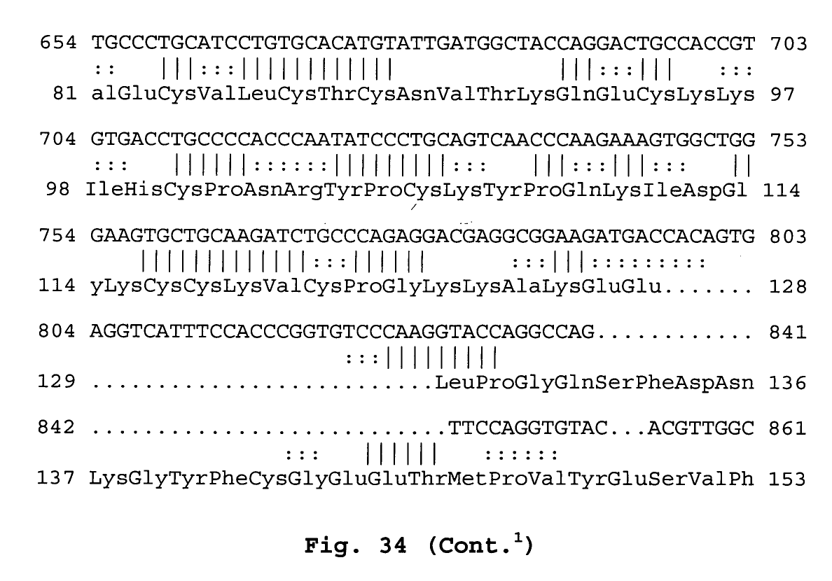

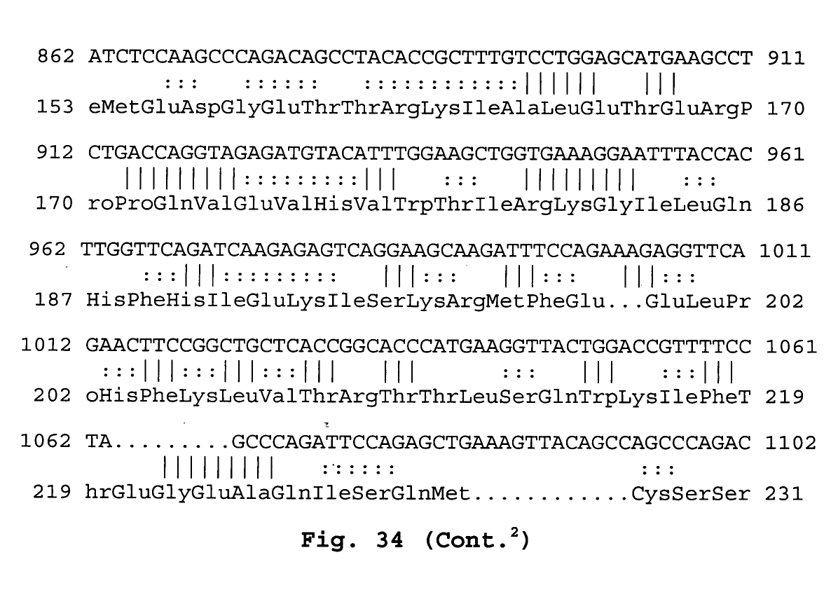

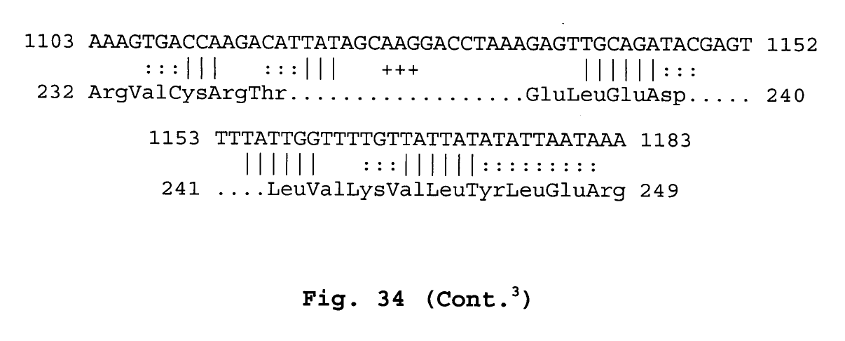

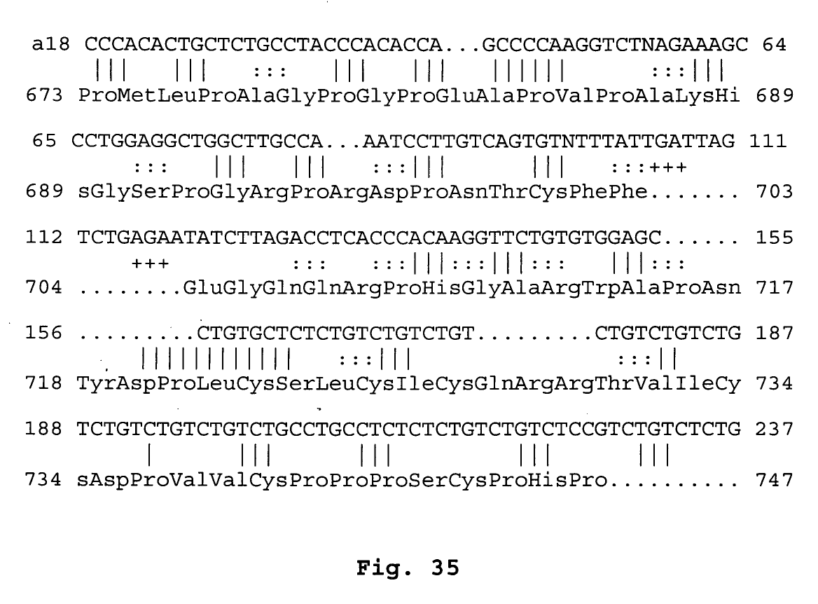

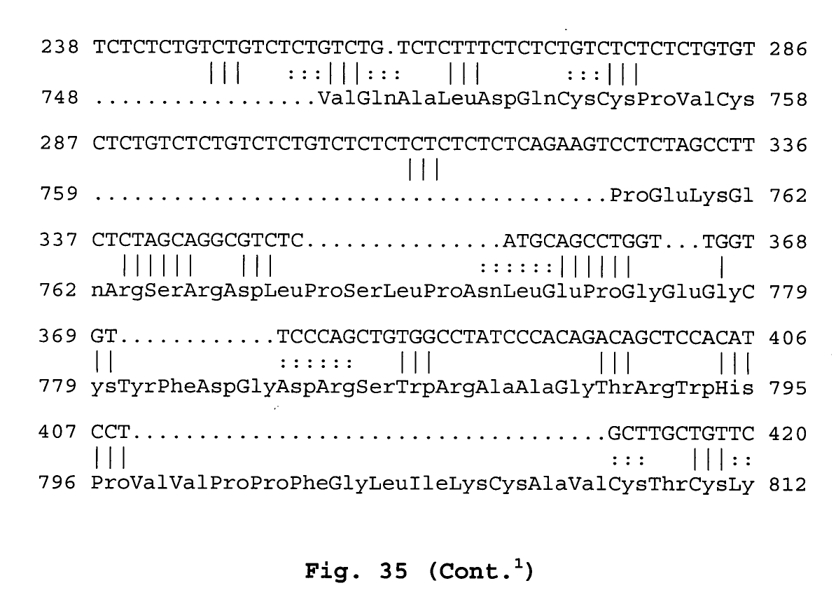

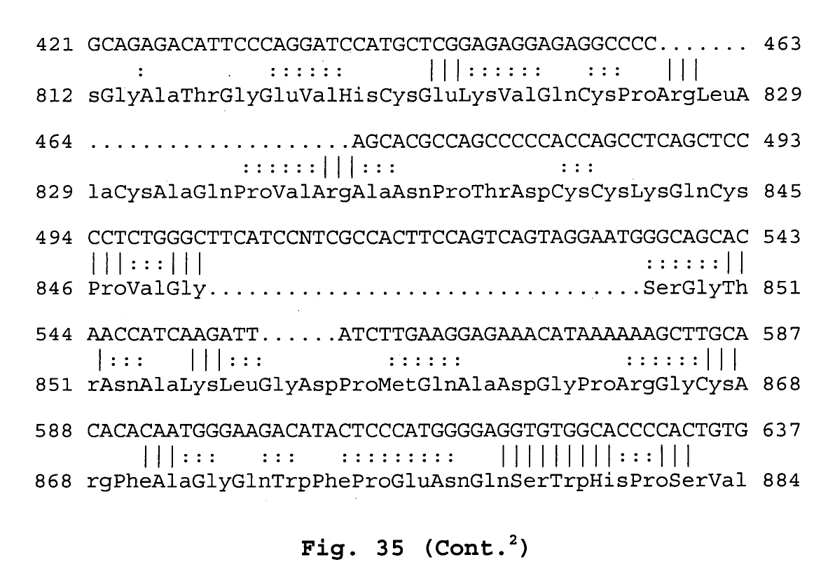

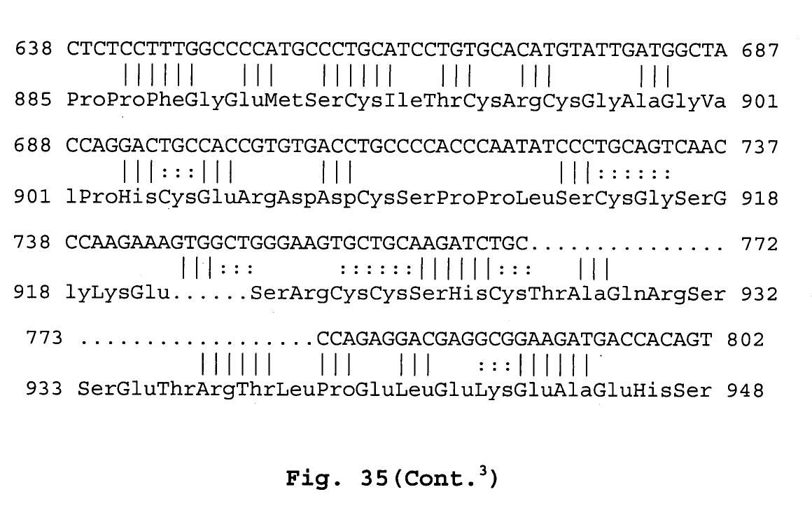

The present invention further provides a protein or polypeptide comprising or consisting of an amino acid sequence encoded by any of the above nucleic acid sequences, termed herein “CHL2 product”, for example, an amino acid sequence having the sequence as in any one of SEQ ID NO: 85 to 95, fragments of the above amino acid sequence having a length of at least 10 amino acids, as well as homologs of the amino acid sequences of any one of SEQ ID NO: 85 to 95 in which one or more of the amino acid residues has been substituted (by conservative or non-conservative substitution) added, deleted, or chemically modified. The comparison between the CHL2 variants SEQ ID NOs 85-95 and other known Chordin related proteins is described in FIGS. 15-35. FIG. 33 shows multiple alignment of the CHL2 variants of the present invention, SEQ ID NOs: 85-95. The sequence of the mouse orthologue of human CHL2 is given by SEQ ID NO: 96.

The present invention further provides nucleic acid molecule comprising or consisting of a sequence which encodes the above amino acid sequences, (including the fragments and analogs of the amino acid sequences). Due to the degenerative nature of the genetic code, a plurality of alternative nucleic acid sequences, beyond SEQ ID NO:74 to SEQ ID NO: 84, can code for the amino acid sequence of the invention. Those alternative nucleic acid sequences which code for the same amino acid sequences codes by the sequences of SEQ ID NO: 74 to SEQ ID NO: 84 are also an aspect of the of the present invention.

The present invention further provides expression vectors and cloning vectors comprising any of the above nucleic acid sequences, as well as host cells transfected by said vectors.

The present invention still further provides pharmaceutical compositions comprising, as an active ingredient, said nucleic acid molecules, said expression vectors, or said protein or polypeptide.

By a second aspect, the present invention provides a nucleic acid molecule comprising or consisting of a non-coding sequence which is complementary to that of any one of SEQ ID NO: 74 to SEQ ID NO: 84, or complementary to a sequence having at least 70%, preferably 80%, most preferably 90% or 95% identity to said sequence or a fragment of said two sequences. The complementary sequence may be a DNA sequence which hybridizes with any one of the sequences of SEQ ID NO: 74 to SEQ ID NO: 84, or hybridizes to a portion of that sequence having a length sufficient to inhibit the transcription of the complementary sequence. The complementary sequence may be a DNA sequence which can be transcribed into an mRNA being an antisense to the mRNA transcribed from any one of SEQ ID NO: 74 to SEQ ID NO: 84 or into an mRNA which is an antisense to a fragment of the mRNA transcribed from any one of SEQ ID NO: 74 to SEQ ID NO: 84 which has a length sufficient to hybridize with the mRNA transcribed from any one of SEQ ID NO: 74 to SEQ ID NO: 84, so as to inhibit its translation. The complementary sequence may also be the mRNA or the fragment of the mRNA itself. The pharmaceutical compositions of the invention (according to both aspects) may be used for the treatment of a plurality of diseases.

In accordance with the present invention, it has been found that the CHL2 of the invention is located in astrocytes. As astrocytes are known to have a variety of physiological activities in maintaining normal brain physiology, such as in the secretion of active compounds, formation of the blood-brain barrier, metabolism of neurotransmitters and maintenance of the ionic balance of the extracellular space. Pharmaceutical compositions in accordance with the present invention may be used to treat diseases and pathological conditions which can be benefited by a modulation of astrocyte activity, such as the modulation of the cross-talk signals in the CNS during physiological and pathological conditions of the nervous system. Examples of such diseases are neuro-degenerative diseases caused by aging, infectious agents, by toxic substances or due to genetic causes. In addition, the pharmaceutical compositions may be used for the treatment of diseases, and pathological conditions involving abnormal development of the nervous system.

It has been postulated that chordin may be expressed by cells of the osteoblastic lineage to limit BMP actions in the osteoblast. This would be a critical function for a BMP binding protein since excessive BMP-4 has been implicated in pathogenesis of fibrodysplasia ossificans progressive Fibrodysplasia Ossificans Progressiva (FOP) is a rare genetic disease in which muscles, tendons, ligaments and other connective tissues may ossify into bone. BMPs can cause induction of noggin and chordin mRNA and protein levels in skeleton cells by transcriptional mechanisms, and in turn these prevent the effect of BMPs in osteoblast in a negative-type feedback. The induction of these proteins by BMPs appears to be a mechanism to limit the BMP effect in bones. Existing therapies which are being investigated for their effectiveness in preventing heterotopic bone formation include BMP inhibitors.

Considerable evidence exists supporting a role for TGF in morphogenesis, in the regulation of endochondral ossification and in bone remodeling. TGF effect the proliferation and differentiation of osteoblastic cells in vitro and high levels of messenger RNA are expressed in the growth plate of fetal human long bones.

The CHL2 of the invention was found by immunohistochemical methods to be localized in fetal-human bone.

Thus, the pharmaceutical compositions of the present invention may be used for the treatment of diseases and pathological conditions associated with osteoblastists or other diseases of mesanchimal origin. An example of such diseases is Fibrodysplasia Ossificans, as well as other diseases involving abnormal bone or cartilage formation, metabolism and/or destruction.

Furthermore, the CHL2 variances of the invention were mapped to chromosome 11q14 (genomic clone accession no. APOO 2010; AP001324; ACO118686).

The chromosomal location of the CHL2 gene is near locations associated with several disorders of cartilage and bone formation, and thus, the pharmaceutical compositions of the invention may be used for the treatment or alleviation of the following diseases:

Osteopetrosis, Autosomal Recessive (congenital disorder characterized also by development of abnormally dense bones).

High Bone Mass (HBM)—High bone mass can result from osteosclerosis (increased density of trabecular—spongy bone) and/or hyperostosis (thickening of cortical—compact bone from deposition of osseous tissue) along subperiosteal and/or endosteal surfaces), occurring focally or throughout the skeleton.

The pharmaceutical compositions of the invention may be used also for the treatment of osteoporosis pseudoglioma syndrome, autosomal recessive osteopetrosis, and isolated increased bone mass (high bone mass without other clinical features). The CHL2 of the invention may also be used for augmenting bone regeneration after injury, so as to speed up the healing process.

In accordance with the findings of the present invention, CHL2 of the invention is expressed in the placenta, and is localized in the uterus lining (endometrium). It is known, that poor preparation of the endometrium (uterine lining) has been associated with abnormal pregnancies and a high rate of miscarriage, as well as other disorders of the female reproductive tract. Thus, the pharmaceutical compositions of the invention may be used for the support of a normal pregnancy, as well as for the treatment of abnormal pregnancies, recurring miscarriages, or the malfunction of the female reproductive tract.

Furthermore, the expression of CHL2 of the invention has also been found to be located in the mullerian epithet in the internal female ganglia (fallopian tubes, uterus, endocervix glands). The CHL2 of the invention can be used to regulate sexual differentiation, for example, by interaction with Mullerian inhibitory substances (MLS), substances secreted by the testes, which cause the regression of the Mullerian duct system in females, leading to female sterility. In addition, the CHL2 of the invention may be used for the treatment of the Lawrence-Moon-Bardet-Biedl syndrome, a rare inherited condition, with variable expression, one of which is hypogenitalism (underdeveloped genitals).

In accordance with another finding of the invention, CHL2 was found to be expressed in tumors of the uterus, prostate and breast, indicating that CHL2 may be a proliferative agent on cell lines in general and tumor cell lines in particular. Thus, pharmaceutical compositions comprising an agent which decreased the expression or level of CHL2, such as in anti-sense therapy, or antibodies, may be used for the treatment of these tumors.

The CHL2 of the invention is a hormone-responsive element, as it expressed in the Mullerian epithelium, ductal epithelium of the breast, prostate, all of which are tissues under sexual hormonal control. Thus, since CHL2 is expressed in all estrogen target tissues (and some androgen target tissues), the pharmaceutical compositions of the invention may be used for hormonal regulation in such pathological conditions, involving non-normal amounts or a non-normal response to sexual hormones.

Pharmaceutical compositions of the invention may also be used for the treatment of cardiovascular disorders.

The nucleic acids of the invention may be used for therapeutic or diagnostic applications, for example, for the detection of the expression of CHL2 in various tissues, as mentioned above (for example, tumors, astrocytes, bone, tissues of the reproductive tract, etc.), and for the detection of any one of its diseases mentioned above. In addition, the ratio between the level of each of the chordin-like homologs to the other may also be indicative of a plurality of physiological or pathological conditions, for example, any one of the diseases mentioned above.

The CHL2 gene of the invention was mapped to genomic locus 11q14, a region containing many potential candidate bone diseases, neural system-related diseases, hormone-dependent diseases and developmental disorders. Thus, the detection of any of the CHL2 of the invention, as well as the detection of their amount or their ratio to each other, may be indicative to the presence of a disease, or a predisposition to a disease, or may be indicative of the severity of the disease. Furthermore, due to said association of the CHL2 of the invention with said disease, the pharmaceutical compositions of the invention (in connection with both aspects, i.e., both the nucleic acid sequence, the anti-sense, the amino acid sequence or the antibody) may be used for the treatment of said diseases or alleviation of some of their side effects.

The following is a list of diseases associated with the same genomic locus as the CHL2 of the invention—which may be detected by the nucleic acid and amino acid sequences of the invention and the antibodies the invention and treated by the pharmaceutical compositions of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An isolated nucleic acid sequence selected from:
(i) the nucleic acid sequence comprising in any one of SEQ ID NO: 1 to: 10 or 74-84;
(ii) nucleic acid sequences having at least about 80%, homology to the sequence of any one of SEQ ID NO: 1 to: 10 or 74-84;
(iii) fragments of (i) or (ii) of at least 20 nucleotides provided that the fragment is not completely identical to a continuous stretch of 20 nucleotides of a nucleotide sequence according to accession number AX175130.

2. An Isolated oligonucleotide according to claim 1 having at least 90% homology to the sequence of any one of SEQ ID NO: 1 to 10 or 74-84.

3. An Isolated oligonucleotide according to claim 1 having at least 95% homology to the sequence of any one of SEQ ID NO: 1 to: 10 or 74-84.

4. A nucleotide fragment according to claim 1 (iii) selected from the group consisting of:
(i) a fragment being at least about 70%, homologous to a portion of SEQ ID NO:1 corresponding to a bridge between exons 9 and 10, said bridge including at least about 20 to 100 nucleotides of exons 9 and 10;
a fragment being at least about 70% homologous to a portion of SEQ ID NO:2 corresponding to a bridge between exons 7 and 9, said bridge including at least about 20 to 100 nucleotides of exons 7 and 9;
a fragment being at least about 70% homologous to a portion of SEQ ID NO:3 corresponding to a bridge between exons 2 and 4 or 9 and 10, said bridge including at least about 20 to 100 nucleotides of exons 2 and 4 or 9 and 10;
a fragment being at least about 70% homologous to a portion of SEQ ID NO:4 corresponding to a bridge between exons 4 and 6, said bridge including at least about 20 to 100 nucleotides of exons 4 and 6;
a fragment being at least about 70% homologous to a portion of SEQ ID NO:5 corresponding to a bridge between exons 2 and 4 or 4 and 6, said bridge including at least about 20 to 100 nucleotides of exons 2 and 4 or 4 and 6.

5. An isolated nucleic acid sequence complementary to the nucleic acid sequence of claim 1.

6. An amino acid sequence selected from:
(i) an amino acid sequence coded by the isolated nucleic acid sequence defined under (i) in claim 1;
(ii) fragments of the amino acid sequence of (i) of at least 10 amino acids provided that the fragment is not completely identical to a continuous stretch of 10 amino acids of the nucleotide sequence given in accession number AX175130;
(iii) analogues of the amino acid sequences of (i) in which one or more amino acids has been added, deleted, replaced or chemically modified without substantially altering the biological activity of the unaltered amino acid sequence.

7. An amino acid sequence selected from:
(i) an amino acid sequence having the sequence of any one of SEQ ID NO: 11 to 20 or 85 to 95,
(ii) an amino acid sequence having at least about 80%, homology to the sequence of (i) wherein said amino acid sequence is less than about 90% homologous to the nucleotide sequence given in accession number AX175130; or
(iii) a portion of a unique region of any of SEQ ID NOS 11-15 selected from:
a. a bridge portion of SEQ ID NO: 11, comprising a peptide having a length “n”, wherein n is at least about 10 to about 50 amino acids in length, wherein at least two amino acids comprise KG, having a structure as follows (numbering according to SEQ ID NO: 11): a sequence starting from any of amino acid number 373-x to 373; and ending at any of amino acid numbers 374+((n−2)−x), in which x varies from 0 to n-2;
b. a bridge portion of SEQ ID NO: 12, comprising a peptide having a length “n”, wherein n is at least about 10 to about 50 amino acids in length, wherein at least two amino acids comprise KE, having a structure as follows (numbering according to SEQ ID NO:12): a sequence starting from any of amino acid number 250-x to 250; and ending at any of amino acid numbers 251+((n−2)−x), in which x varies from 0 to n-2;
c. a bridge portion of SEQ ID NO: 13, comprising a peptide having a length “n”, wherein n is at least about 10 to about 50 amino acids in length, wherein at least two amino acids comprise EN, having a structure as follows (numbering according to SEQ ID NO:13): a sequence starting from any of amino acid number 45-x to 45; and ending at any of amino acid numbers 46+((n−2)−x), in which x varies from 0 to n-2; wherein if the peptide is 50 amino acids in length, the starting position cannot be any smaller than 1;
d. a bridge portion of SEQ ID NO: 14, comprising a peptide having a length “n”, wherein n is at least about 10 to about 50 amino acids in length, wherein at least two amino acids comprise TM, having a structure as follows (numbering according to SEQ ID NO:14): a sequence starting from any of amino acid number 124-x to 124 and ending at any of amino acid numbers 125+((n−2)−x), in which x varies from 0 to n-2, wherein the ending position is not greater than 142;
e. a bridge portion of SEQ ID NO: 15, comprising a peptide having a length “n”, wherein n is at least about 10 to about 50 amino acids in length, wherein at least two amino acids comprise EN, having a structure as follows (numbering according to SEQ ID NO:15): a sequence starting from any of amino acid number 45-x to 45; and ending at any of amino acid numbers 46+((n−2)−x), in which x varies from 0 to n-2; wherein if the peptide is 50 amino acids in length, the starting position cannot be any smaller than 1;
(iv) a portion of an amino acid sequence, having at least about 80% homology to a portion of SEQ ID NOs 11-15 defined in (iii) or (v).

8. An amino acid sequence according to claim 7(iii) wherein the homology is at least about 90%.

9. An amino acid sequence according to claim 7(iii) wherein the homology is at least about 95%.

10. An antibody capable of specifically binding to at least one epitope of any of the amino acid sequences of claim 6.

11. The antibody of claim 10, wherein said selectivity of said antibody is at least two fold higher for an epitope of a sequence of
(i) an amino acid sequence coded by the isolated nucleic acid selected from:
(a) the nucleic acid sequence comprising in any one of SEQ ID NO: 1 to: 10 or 74-84
(b) nucleic acid sequences having at least about 80%, homology to the sequence of any one of SEQ ID NO: 1 to: 10 or 74-84:
(c) fragments of (i) or (ii) of at least 20 nucleotides provided that the fragment is not completely identical to a continuous stretch of 20 nucleotides of a nucleotide sequence according to accession number AX175130;
(ii) fragments of the amino acid sequence of (i) of at least 10 amino acids provided that the fragment is not completely identical to a continuous stretch of 10 amino acids of the nucleotide sequence given in accession number AX 175130;
(iii) analogues of the amino acid sequences of (i) in which one or more amino acids has been added, deleted, replaced or chemically modified without substantially altering the biological activity of the unaltered amino acid sequence than for an epitope present in a protein having a sequence according to a nucleotide sequence given in accession number AX175130.

12. An isolated mRNA or cDNA nucleic acid sequence coding for the amino acid sequence of claim 6.

13. An expression vector comprising the nucleic acid sequences of claim 1 and a control element for the expression of the nucleic acid sequence in a suitable host.

14. An expression vector comprising the nucleic acid sequences of claim 12, and a control element for the expression of the nucleic acid sequence in a suitable host.

15. An expression vector comprising the nucleic acid sequence of claim 5, and control elements for the expression of the nucleic acid sequence in a suitable host.

16. A host cell transfected by the expression vector of claim 13.

17. A pharmaceutical composition comprising a pharmaceutically acceptable carrier and, as an active ingredient, the expression vector of claim 13.

18. A pharmaceutical composition comprising a pharmaceutically acceptable carrier and as the active ingredient at least one nucleic acid of claim 1.

19. A pharmaceutical composition according to claim 18 further comprising an agent for facilitation of nucleic acid uptake by cells.

20. A pharmaceutical composition comprising a pharmaceutically acceptable carrier and, as an active ingredient, the amino acid sequence of claim 6.

21. A pharmaceutical composition comprising a pharmaceutically acceptable carrier as an active ingredient the nucleic acid sequence of claim 5.

22. A pharmaceutical composition comprising a pharmaceutically acceptable carrier as an active ingredient the expression vector of claim 15.

23. A pharmaceutical composition comprising a pharmaceutically acceptable carrier as an active ingredient a purified antibody of which binds specifically to the amino acid sequence of claim 6.

24. A method for treating a disease selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut; the method comprising administering to a subject in need an active ingredient being an expression vector comprising a nucleic acid sequence and a control element for the expression of the nucleic acid sequence in a suitable host, the nucleic acid sequence selected from:
(i) the nucleic acid sequence of claim 1;
(ii) the nucleic acid sequence comprising in any one of SEQ ID NO: 1 to: 10 or 74-84;
(iii) nucleic acid sequences having at least about 80% homology to the sequence of any one of SEQ ID NO: 1 to 10 or 74-84;
(iv) fragments of (ii) or (iii) of at least 20 nucleotide.

25. A method according to claim 24 wherein the homology of (ii) is at least 90%.

26. A method according to claim 25 wherein the homology is at least 95%.

27. A method for treating a disease selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut; the method comprising administering to a subject in need an active ingredient being an amino acid sequence selected from:
(i) the amino acid sequence of claim 11;
(ii) an amino acid sequence having the sequence of any one of SEQ ID NO: 11 to 20 or SEQ 85 to 95;
(iii) an amino acid sequence having at least about 80%, homology to the sequence of any one of SEQ ID NO: 11 to 20 or SEQ 85 to 95
(iv) a portion of an amino acid sequence of (ii) or (iii).

28. A method according to claim 27 wherein the homology of (ii) is at least 90%.

29. A method according to claim 27 wherein the homology of (ii) is at least 95%.

30. A method for treating a disease selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut; the method comprising administering to a subject in need an active ingredient, ingredient, optionally together with an agent for facilitating uptake of nucleic acids by cells, the active ingredient being, a nucleic acid sequence selected from:
(i) the nucleic acid sequence of claim 1;
(ii) the nucleic acid sequence comprising in any one of SEQ ID NO: 1 to: 10 or 74-84;
(iii) nucleic acid sequences having at least about 80% homology to the sequence of any one of SEQ ID NO: 1 to 10 or 74-84;
(iv) fragments of (ii) or (iii) of at least 20 nucleotides.

31. A method according to claim 30 (iii) wherein the homology is at least 90%.

32. A method according to claim 30(iii) wherein the homology is at least 95%.

33. A method for treating a disease selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut; the method comprising administering to a subject in need an active ingredient being the nucleic acid sequence of claim 5.

34. A method according to claim 33 wherein the nucleic acid sequence is small interfering RNA (siRNA).

35. A method for treating a disease selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut; the method comprising administering to a subject in need an active ingredient being an expression vector comprising a nucleic acid sequence according to claim 5 and a control element for the expression of the nucleic acid sequence in a suitable host.

36. A method for treating a disease selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut; the method comprising administering to a subject in need an active ingredient being a purified antibody which binds specifically to the amino acid sequence of claim 6.

37. A method for detecting an CHL2 nucleic acid sequence in a biological sample, comprising:
(a) hybridizing to nucleic acid material of said biological sample any one of the nucleic acid sequence of claim 1 or a nucleic acid sequence complementary thereto; and
(b) detecting said hybridization complex;
wherein the presence of said hybridization complex correlates with the presence of an CHL2 nucleic acid sequence in the said biological sample.

38. A method according to claim 37, wherein the nucleic acid material of said biological sample are mRNA transcripts.

39. A method according to claim 38, where the nucleic acid sequence is present in a nucleic acid chip.

40. A method for detection of a nucleic acid sequence in a biological sample, comprising the steps of:
(i) providing a probe comprising at least one of the nucleic acid sequence of claim 1 to 4 or a sequence complementary thereto; and
(ii) contacting the biological sample with said probe under conditions allowing hybridization of nucleic acid sequences thereby enabling formation of hybridization complexes; and
detecting hybridization complexes, wherein the presence of the complex indicates the presence of nucleic acid sequence encoding the CHL2 product in said biological sample.

41. A method for detecting antibodies in a biological sample comprising:
(a) contacting said biological sample with an amino acid sequence of claim 6, thereby forming an antibody-antigen complex; and
(b) detecting said antibody-antigen complex,
wherein the presence of said antibody-antigen complex correlates with the presence of anti-CHL2 antibody in said biological sample.

42. A biomarker for detecting prostate cancer, comprising hCHL2 variant X (SEQ ID No 10) or a fragment thereof.

43. The biomarker of claim 42, wherein said fragment comprises hCHL2 exon 4a.

44. A primer pair for use in detecting the biomarker of claim 42, comprising a primer pair capable of amplifying hCHL2 variant X (SEQ ID 10) or a fragment thereof.

45. The primer pair of claim 46, comprising hCHL2 exon 4a-forward primer: AAACCTCATTTTCTTCTTCCTCCTG(SEQ ID NO: 68.); and hCHL2 exon 4a -Reverse primer:
CTGAAGATCTCTCCGTGTTGGTACATG_(SEQ ID NO:69).

46. An amplicon obtained through the use of a primer pair according to claim 44.

47. The amplicon of claim 31, comprising hCHL2 exon 4a amplicon_(SEQ ID NO:70): having the following sequence:
AAACCTCATTTTCTTCTTCCTCCTGCCCCTCCCCCACTGCAGAACCTCAC
ACTCCCTCTGGACTCCGGGCCCCACCAAAGTCCTgCCAGCACAACG GGA
CCATGTACCAACACGGA GAGATCTTCAG.

48. A nucleic acid molecule capable of selectively hybridizing to hCHL2 variant X (SEQ ID NO: 10) or a fragment thereof.

49. A biomarker for detection of cancers comprising the sequence of exon 2a (SEQ ID no:64).

50. A kit for detecting prostate cancer, comprising:
(a) a probe for hybridizing to hCHL2 variant X (SEQ ID NO 10) or fragment thereof, optionally linked to a detectable label;
(b) a calibration curve for comparing hybriziation results of a nucleic acid in a sample and the probe to results obtained with the same probe, from control cells and/or prostate cancer cells.

51. A kit for detection of prostate cancer of cells comprising:
(a) primers for amplification of h CHL2 (SEQ ID NO 10) or a fragment thereof;
(b) reagents for an amplifications of nucleic acid sequences.

52. The kit of claim 50, wherein said fragment comprises hCHL2 exon 4a.

53. A method for detecting prostate cancer, in cells suspected of being cancerous, comprising:
(1) determining the level of expression of hCHL2 variant X or a fragment thereof;
(2) comparing the level of (1) to the level of expression in a control healthy cells;
a significantly higher level of expression indication prostate cancer in the cells.

54. The method of claim 53 wherein the level of expression is determined by detecting the level of protein depicted by SEQ ID: 20 or a fragment thereof

55. The method of claim 53 wherein the level of expression is determined by detecting the level of the mRNA of hCHL2 variant X as depicted in SEQ ID NO: 10 or a fragment thereof.

56. The method of claim 55 performed by using NAT-based technology.

57. The method of claim 52, wherein said fragment comprises hCHL2 exon 4a.

58. A biomarker for detecting prostate cancer, comprising CHL2 variant X amino acid sequence according to SEQ ID NO: 20 or a fragment thereof.

59. A biomarker for detecting breast or lung cancer, comprising any one of hCHL2 variants IV, V, VI, VII, VIII or IX (SEQ ID NOs: 3, 4, 5, 7, 8, 9 respectively ) or a fragments thereof.

60. The biomarker of claim 58, wherein said fragment comprises hCHL2 exon 2a.

61. The biomarker of claim 58, wherein said fragment comprises hCHL2 amplicon of SEQ ID NO: 64.

62. A primer pair for use in detecting the biomarker of claim 58, comprising a primer pair capable of amplifying hCHL2 variants IV, V, VI, VII, VIII and IX (SEQ ID NOs: 3, 4, 5, 7, 8, 9) or a fragment thereof.

63. The primer pair of claim 62, comprising hCHL2 exon 2a-forward primer: AACATGGCACTGGTCGGTTT (SEQ ID NO: 62); and hCHL2 exon 2a -Reverse primer (SEQ ID NO: 63):
CGGACAGTGGAGGCGGTA.

64. An amplicon obtained through the use of a primer pair according to claim 62.

65. The amplicon of claim 64, comprising hCHL2 exon 2a amplicon: (SEQ ID NO:64)
AACATGGCACTGGTCGGTTTGCCAGGCCCAGACATGTTCTGCCTTT
TCCATGGGAAGAGATACTCCCCCGGCGAGAGCTGGCACCCCTACTTGGAG
CCACAAGGCCTGATGTACTGCCTGCGCTGTACCTGCTCAGAGGGCGCCCA
TGTGAGTTGTTACCGCCTCCACTGTCCG.

66. A nucleic acid molecule capable of selectively hybridizing to any one of the hCHL2 variants IV, V, VI, VII, VIII or IX (SEQ ID NOs: 3, 4, 5, 7, 8, 9) or a fragment thereof.

67. A method for detecting lung or breast cancer in cells suspected of being cancerous, comprising:
(a) determining the level of expression of any one of hCHL2 variants IV, V, VI, VII, VIII or IX (SEQ ID NOs: 3, 4, 5, 7, 8, 9) or a fragment thereof;
(b) comprising the level of expression obtained in (a) to that of control cells; a significantly higher level of expression indicates lung or breast cancer.

68. The method of claim 67 wherein the level of expression is carried out by determining the mRNA level.

69. The method of claim 68 wherein the detection of the mRNA level is carried out using NAT-based technology.

70. The method of claim 67, wherein said fragment comprises hCHL2 exon 2a.

71. The method of claim 67, wherein said fragment comprises hCHL2 exon 2a.

72. A biomarker for detecting lung or breast cancer, comprising any one of CHL2 variant IV, V, VI, VII, VIII or IX amino acid sequence according to any of SEQ ID NOs: 13, 14, 15, 17, 18, 19.

73. A method according to claim 37, wherein detecting a CHL2 nucleic acid sequence in a biological sample can be used for diagnosis of CHL2-related disease.

74. A method according to claim 73, wherein a CHL2-related disease is selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut.

75. A method for detecting CLH2-product in a biological sample, comprising the steps of:
(a) contacting with said biological sample the antibody of claim xxx, thereby forming an antibody-antigen complex; and
(b) detecting said antibody-antigen complex
wherein the presence of said antibody-antigen complex correlates with the presence of CLH2 product in said biological sample.

76. A method according to claim 75, wherein detecting a CHL2 product in a biological sample can be used for diagnosis of CHL2-related disease.

77. A method according to claim 75, wherein a CHL2-related disease is selected from: diseases manifested in non-normal bone formation and non-normal bone modeling; bone injuries; diseases involved with the female reproductive tract; diseases of disorders involved with abnormal sexual differentiation; recurrent miscarriages, tumors of the lung, uterus, breast or prostate; diseases involving sexual hormone abnormalities; cardiovascular disorders; neuronal diseases of the CNS; neuro-degenerative diseases and diseases involving non-normal developments of neurons; inflammatory diseases; diseases of the gut.


SEQUENCE LISTING (95 sequences)

SEQ ID NO: 1 - DNA, 1667 bp, Homo sapiens
accttccccctttctttgatcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcgggctcacaacctgccccgaaccaggctgcccagcacccctcccgctgccagactcctgctgccaggcctgcaaagatgaggcaagtgagcaatcggatgaagaggaccgtgtgcagtcgctccatggggtgagacatcctcaggatccatgttccagtgatgctgggagaaagagaggcccgggcaccccagcccccactggcctcagcgcccctctgagcttcatccctcgccacttcatacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacctggtggagatctacctctggaagctggtaaaaggaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgctgttattatatattaataaataagaagttgcataaccatcaaaa

SEQ ID NO: 2 - DNA, 1219 bp, Homo sapiens
accttccccctttctttgatcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcgggctcacaacctgccccgaaccaggctgcccagcacccctcccgctgccagactcctgctgccaggcctgcaaagatgaggcaagtgagcaatcggatgaagaggaccgtgtgcagtcgctccatggggtgagacatcctcaggatccatgttccagtgatgctgggagaaagagaggcccgggcaccccagcccccactggcctcagcgcccctctgagcttcatccctcgccacttcatacccaagggagcaggcagcacaactgtcaagatcgtcctgaaggagaaacataagaaagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacctggtggagatctacctctggaagctggtaaaag

SEQ ID NO: 3 - DNA, 1415 bp, Homo sapiens
ggacaaataaaaaggaaacaagcatgattgtgagggcagaggagcgtgggactgagtcaggagactggtgctgtcatcgctgcctggtgactgacttgctgtgtggccctcaggtgtaacttaccctctctgggcctcatttgtctaatcataataattaacgctgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgcctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaag

SEQ ID NO: 4 - DNA, 1415 bp, Homo sapiens
ggacaaataaaaaggaaacaagcatgattgtgagggcagaggagcgtgggactgagtcaggagactggtgctgtcatcgctgcctggtgactgacttgctgtgtggccctcaggtgtaacttaccctctctgggcctcatttgtctaatcataataattaacgctgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgcctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacaatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaag

SEQ ID NO: 5 - DNA, 1321 bp, Homo sapiens
ggacaaataaaaaggaaacaagcatgattgtgagggcagaggagcgtgggactgagtcaggagactggtgctgtcatcgctgcctggtgactgacttgctgtgtggccctcaggtgtaacttaccctctctgggcctcatttgtctaatcataataattaacgctgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgcctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacaatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaag

SEQ ID NO: 6 - DNA, 1722 bp, Homo sapiens
accttccccctttctttgatcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcgggctcacaacctgccccgaaccaggctgcccagcacccctcccgctgccagactcctgctgccaggcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacctggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgctgttattatatattaataaataagaagttgcataaccatcaaaa

SEQ ID NO: 7 - DNA, 1817 bp, Homo sapiens
ggacaaataaaaaggaaacaagcatgattgtgagggcagaggagcgtgggactgagtcaggagactggtgctgtcatcgctgcctggtgactgacttgctgtgtggccctcaggtgtaacttaccctctctgggcctcatttgtctaatcataataattaacgctgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgcctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 8 - DNA, 1622 bp, Homo sapiens
ggacaaataaaaaggaaacaagcatgattgtgagggcagaggagcgtgggactgagtcaggagactggtgctgtcatcgctgcctggtgactgacttgctgtgtggccctcaggtgtaacttaccctctctgggcctcatttgtctaatcataataattaacgctgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgcctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 9 - DNA, 1567 bp, Homo sapiens
ggacaaataaaaaggaaacaagcatgattgtgagggcagaggagcgtgggactgagtcaggagactggtgctgtcatcgctgcctggtgactgacttgctgtgtggccctcaggtgtaacttaccctctctgggcctcatttgtctaatcataataattaacgctgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgcctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaaggaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 10 - DNA, 1515 bp, Homo sapiens
agaacagtgcctggcactgagaagtgctccatgaaccctcattaaatgctggatgaactaactcgggccccactgagcggaagctgaggctgccggctttaccttcttgctctggttggggctggcccagacactgagggggctggagggctgtggtagaggtcatgggagggagggactcagtcagatgtaggtatcagagggacctcttagttagctgataaggggaatggctggcaaggcccaggccagagcttggtttaaatatcaagctggggtcaaatgcaaaatcatcagaaaagtggccttgttaatttcagcaaagattcacatgaaacctcattttcttcttcctcctgcccctcccccactgcagaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaggcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 11 - protein, 429 aa, Homo sapiens
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKGIFHLTQIKKVRKQDFQKEAQHFRLLAGPHEGHWNVFLAQTLELKVTASPDKVTKT

SEQ ID NO: 12 - protein, 308 aa, Homo sapiens
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKEDKADPGHSEISSTRCPKAPGRVLVHTSVSPSPDNLRRFALEHEASDLVEIYLWKLVK

SEQ ID NO: 13 - protein, 142 aa, Homo sapiens
MALVGLPGPDMFCLFHGKRYSPGESWHPYLEPQGLMYCLRCTCSENLTLPLDSGPHQSPASTTGPCTNTERSSVPMSCSPPACPTSVSSAAAQRARSTAASQPAPNQAAQHPSRCQTPAAKPAKMRQVSNRMKRTVCSRSMG

SEQ ID NO: 14 - protein, 142 aa, Homo sapiens
MALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTMRQVSNRMKRTVCSRSMG

SEQ ID NO: 15 - protein, 93 aa, Homo sapiens
MALVGLPGPDMFCLFHGKRYSPGESWHPYLEPQGLMYCLRCTCSENLTLPLDSGPHQSPASTTGPCTNTERSSVPMSCSPPACPTSVSSAAAQ

SEQ ID NO: 16 - protein, 451 aa, Homo sapiens
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDRVQSLHGVRHPQDPCSSDAGRKRGPGTPAPTGLSAPLSFIPRHFIPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 17 - protein, 431 aa, Homo sapiens
MALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 18 - protein, 366 aa, Homo sapiens
MALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 19 - protein, 344 aa, Homo sapiens
MALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKEDKADPGHSEISSTRCPKAPGRVLVHTSVSPSPDNLRRFALEHEASDLVEIYLWKLVKGIFHLTQIKKVRKQDFQKEAQHFRLLAGPHEGHWNVFLAQTLELKVTASPDKVTKT

SEQ ID NO: 20 - protein, 386 aa, Homo sapiens
MQNHQKSGLVNFSKDSHETSFSSSSCPSPTAEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 21 - DNA, 23 bp, Homo sapiens
cagcggcctgaccctggggaaag

SEQ ID NO: 22 - DNA, 24 bp, Homo sapiens
ctgcttgattccctaactgccctg

SEQ ID NO: 23 - DNA, 23 bp, Homo sapiens
cagagggacctcttagttagctg

SEQ ID NO: 24 - DNA, 27 bp, Homo sapiens
ctgaagatctctccgtgttggtacatg

SEQ ID NO: 25 - DNA, 23 bp, Homo sapiens
cagtgatgctgggagaaagagag

SEQ ID NO: 26 - DNA, 24 bp, Homo sapiens
agggtctgctttgtcctctttctt

SEQ ID NO: 27 - DNA, 27 bp, Homo sapiens
cacctggtagaactgatctcactgtgg

SEQ ID NO: 28 - DNA, 23 bp, Homo sapiens
agctcatatctgcaactgttagg

SEQ ID NO: 29 - DNA, 21 bp, Homo sapiens
gaaagcctgtgtgcatggcgg

SEQ ID NO: 30 - DNA, 23 bp, Mouse
atctcactttctcccggctcatc

SEQ ID NO: 31 - DNA, 24 bp, Mouse
gttgcgcattcttgtattcaggag

SEQ ID NO: 32 - DNA, 23 bp, Mouse
atcacaggctgtgagcagtgaag

SEQ ID NO: 33 - DNA, 23 bp, Mouse
ttcaggacaggtcatgagaccac

SEQ ID NO: 34 - DNA, 22 bp, Mouse
aggatccatgctcggagaccag

SEQ ID NO: 35 - DNA, 22 bp, Mouse
catgctccaggacaaagcggtg

SEQ ID NO: 36 - DNA, 20 bp, Homo sapiens
ctcacactcctcgccctatt

SEQ ID NO: 37 - DNA, 20 bp, Homo sapiens
gactggggctcccagccatt

SEQ ID NO: 38 - DNA, 23 bp, Mouse
gacaagtcccacacagcagcttg

SEQ ID NO: 39 - DNA, 22 bp, Mouse
cagccaaagccgagctgccaga

SEQ ID NO: 40 - DNA, 24 bp, Mouse
acgaaaccatgcccaactgagatt

SEQ ID NO: 41 - DNA, 24 bp, Mouse
agaggctttggaaccggatagctc

SEQ ID NO: 42 - DNA, 25 bp, Homo sapiens
cagtgattataggctttcgctctaa

SEQ ID NO: 43 - DNA, 23 bp, Homo sapiens
cagggctattggttgaatgagta

SEQ ID NO: 44 - DNA, 24 bp, Homo sapiens
tggcattacgaacctttggccact

SEQ ID NO: 45 - DNA, 24 bp, Homo sapiens
cattgcagccagacgggcattatt

SEQ ID NO: 46 - DNA, 22 bp, Mouse
tcgtggagtctactggcgtctt

SEQ ID NO: 47 - DNA, 22 bp, Homo sapiens
tggcagtgatggcatggactgt

SEQ ID NO: 48 - DNA, 1516 bp, Mouse
ctcttcatctttgcttttccgactcctcaagcagcggtcctacttggtcctctgaggacttacttgtgtccttatctcactttctcccggctcatcccggggttgtctgaccttgggacaaggaaggatggttcccggggtgaggatcatcccctctttgctgggactcgtgatgttctggctcccgttggactcacaagcacgatcccgctcgggcaaagtctgccttttcggtgaaaagatatatacccccggccagagctggcacccctacttggaaccacaaggcacgatatactgcgtgcgctgtacctgctctgagaatggacatgtgaattgttaccgcctccgctgcccaccccttcactgctcacagcctgtgatggagccacagcaatgctgtcccaggtgtgtggatcctcatgtcccctctggcctccgagttcccctaaagtcctgccagctcaatgagaccacataccaacatggagagatcttcagtgcccaggagctgttccctgcccgcctgtccaaccagtgtgtcctgtgtagctgtattgaaggccacacttactgtggtctcatgacctgtcctgaacccagctgccccaccacactccctctgcctgattcctgctgtcagacctgcaaagacaggacaactgagagctccacagaagaaaacttgacacagctgcagcatggagagagacattcccaggatccatgctcggagaggagaggccccagcacgccagcccccaccagcctcagctcccctctgggcttcatccctcgccacttccagtcagtaggaatgggcagcacaaccatcaagattatcttgaaggagaaacataaaaaagcttgcacacacaatgggaagacatactcccatggggaggtgtggcaccccactgtgctctcctttggccccatgccctgcatcctgtgcacatgtatcgatggctaccaggactgccaccgtgtgacctgccccacccaatatccctgcagtcaacccaagaaagtggctgggaagtgctgcaagatctgcccagaggacgaggcggaagatgaccacagtgaggtcatttccacccggtgtcccaaggtaccaggccagttccatgtgtacacgttggcatctccaagcccagacagcctacaccgctttgtcctggagcatgaagcctctgaccaggtagagatgtacatttggaagctggtgaaaggaatctaccacttggttcagatcaagagagtcaggaagcaagatttccagaaagaggctcagaacttccggctgctcaccggcacccatgaaggttactggaccgtcttcctagcccagactccagagctgaaagttacagccagcccagacaaagtgaccaagacattatagcaaggacctaaagagttgcagatacgagttttattggttttgttattatatattaataaagaagtcgcattaccctcaaaaaaaaaaaaaaaaaaaaaaaaaaaaaaa

SEQ ID NO: 49 - protein, 426 aa, Mouse
MVPGVRIIPSLLGLVMFWLPLDSQARSRSGKVCLFGEKIYTPGQSWHPYLEPQGTIYCVRCTCSENGHVNCYRLRCPPLHCSQPVMEPQQCCPRCVDPHVPSGLRVPLKSCQLNETTYQHGEIFSAQELFPARLSNQCVLCSCIEGHTYCGLMTCPEPSCPTTLPLPDSCCQTCKDRTTESSTEENLTQLQHGERHSQDPCSERRGPSTPAPTSLSSPLGFIPRHFQSVGMGSTTIKIILKEKHKKACTHNGKTYSHGEVWHPTVLSFGPMPCILCTCIDGYQDCHRVTCPTQYPCSQPKKVAGKCCKICPEDEAEDDHSEVISTRCPKVPGQFHVYTLASPSPDSLHRFVLEHEASDQVEMYIWKLVKGIYHLVQIKRVRKQDFQKEAQNFRLLTGTHEGYWTVFLAQTPELKVTASPDKVTKTL

SEQ ID NO: 50 - DNA, 21 bp, Artificial Sequence
  HPRT1-Forward primer
tgacactggcaaaacaatgca

SEQ ID NO: 51 - DNA, 21 bp, Artificial Sequence
  HPRT1-Reverse primer
ggtccttttcaccagcaagct

SEQ ID NO: 52 - DNA, 94 bp, Artificial Sequence
  HPRT1 amplicon
tgacactggcaaaacaatgcagactttgctttccttggtcaggcagtataatccaaagatggtcaaggtcgcaagcttgctggtgaaaaggacc

SEQ ID NO: 53 - DNA, 19 bp, Artificial Sequence
  PBGD-Forward primer
tgagagtgattcgcgtggg

SEQ ID NO: 54 - DNA, 21 bp, Artificial Sequence
  PBGD-Reverse priimer
ccagggtacgaggctttcaat

SEQ ID NO: 55 - DNA, 91 bp, Artificial Sequence
  PBGD amplicon
tgagagtgattcgcgtgggtacccgcaagagccagcttgctcgcatacagacggacagtgtggtggcaacattgaaagcctcgtaccctgg

SEQ ID NO: 56 - DNA, 20 bp, Artificial Sequence
  SDHA-Forward primer
tgggaacaagagggcatctg

SEQ ID NO: 57 - DNA, 22 bp, Artificial Sequence
  SDHA-Reverse primer
ccaccactgcatcaaattcatg

SEQ ID NO: 58 - DNA, 86 bp, Artificial Sequence
  SDHA amplicon
tgggaacaagagggcatctgctaaagtttcagattccatttctgctcagtatccagtagtggatcatgaatttgatgcagtggtgg

SEQ ID NO: 59 - DNA, 20 bp, Artificial Sequence
  G6PD-Forward primer
gaggccgtcaccaagaacat

SEQ ID NO: 60 - DNA, 19 bp, Artificial Sequence
  G6PD-Reverse primer
ggacagccggtcagagctc

SEQ ID NO: 61 - DNA, 111 bp, Artificial Sequence
  G6PD amplicon
gaggccgtcaccaagaacattcacgagtcctgcatgagccagataggctggaaccgcatcatcgtggagaagcccttcgggagggacctgcagagctctgaccggctgtcc

SEQ ID NO: 62 - DNA, 20 bp, Artificial Sequence
  Exon 2a-Forward primer
aacatggcactggtcggttt

SEQ ID NO: 63 - DNA, 18 bp, Artificial Sequence
  Exon 2a-Reverse primer
cggacagtggaggcggta

SEQ ID NO: 64 - DNA, 174 bp, Artificial Sequence
  amplicon exon 2a
aacatggcactggtcggtttgccaggcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccg

SEQ ID NO: 65 - DNA, 19 bp, Artificial Sequence
  Ubiquitin-Forward primer
atttgggtcgcggttcttg

SEQ ID NO: 66 - DNA, 21 bp, Artificial Sequence
  Ubiquitin-Reverse primer
tgccttgacattctcgatggt

SEQ ID NO: 67 - DNA, 133 bp, Artificial Sequence
  Ubiquitin Amplicon
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 68 - DNA, 25 bp, Artificial Sequence
  Exon 4a Forward primer
aaacctcattttcttcttcctcctg

SEQ ID NO: 69 - DNA, 27 bp, Artificial Sequence
  Exon 4a-Reverse primer
ctgaagatctctccgtgttggtacatg

SEQ ID NO: 70 - DNA, 127 bp, Artificial Sequence
  Exon 4a- amplicon
aaacctcattttcttcttcctcctgcccctcccccactgcagaacctcacactccctctggactccgggccccaccaaagtcctgccagcacaacgggaccatgtaccaacacggagagatcttcag

SEQ ID NO: 71 - DNA, 23 bp, Artificial Sequence
  RPL-19-Forward primer
tggcaagaagaaggtctggttag

SEQ ID NO: 72 - DNA, 22 bp, Artificial Sequence
  RPL-19-Reverse primer
tgatcagcccatctttgatgag

SEQ ID NO: 73 - DNA, 101 bp, Artificial Sequence
  RPL-19 amplicon
tggcaagaagaaggtctggttagaccccaatgagaccaatgaaatcgccaatgccaactcccgtcagcagatccggaagctcatcaaagatgggctgatca

SEQ ID NO: 74 - DNA, 1281 bp, Homo sapiens
  misc_feature (600)..(600): n is any nucleic acid
cagggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacatangaaagcctgtgtgcatggcgggaagacgtactcccacggggaggtgtggcacccggccttccgtgccttcggcccttgcccatgcatcctatgcacctgtgaggatggccgccaggactgccagcgtgtgacctgtcccacgaagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 75 - DNA, 1722 bp, Homo sapiens
  misc_feature (224)..(224): n is any nucleic acid
accttccccctttctttgatcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtcatgtgggttgatctgtggcccctgtgnctccgtgtccttttcgtctcccgtcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacatangaaagcctgtgtgcatggcgggaagacgtactcccacggggaggtgtggcacccggccttccgtgccttcggcccttgcccatgcatcctatgcacctgtgaggatggccgccaggactgccagcgtgtgacctgtcccacgaagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 76 - DNA, 1515 bp, Homo sapiens
  misc_feature (15)..(15): n is any nucleic acid
agaacagtgcctggnactgagaagtgctncatgaaccctcattaaaatgctggatgaactaactcgggccccactgagcggaagctgaggctgccggctttaccttcttgctctggttggggctggcccagacactgagggggctggagggctgtggtagaggtcatgggagggagggactcagtcagatgtaggtatcagagggacctcttagttagctgataaggggaatggctggcaaggcccaggccagagcttggtttaaatatcaagctggggtcaaatgcaaaatcatcagaaaagtggccttgttaatttcagcaaagattcacatgaaacctcattttcttcttcctcctgcccctcccccactgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacatangaaagcctgtgtgcatggcgggaagacgtactcccacggggaggtgtggcacccggccttccgtgccttcggcccttgcccatgcatcctatgcacctgtgaggatggccgccaggactgccagcgtgtgacctgtcccacgaagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 77 - DNA, 1890 bp, Homo sapiens
  misc_feature (4)..(4): n is any nucleic acid
tagnancgnccgccaggggntgnattcgcccttactcannatagggntnaacggccgcccggncaggtgccaaatggacaaataaaaggaaacaagcatgattgtnaggncagaggagggtggnattnagtcagaagactggtgctgtcatcgctgcntggtgactgacttgctgtgtggccntcaggtgtaacttaccctctctgggcctcatttgtctaatcataataattaacgttgataccatgatataaatctgtacagcatttcactgcttgattccctaactgccctgtgagataagcgttaaggctcagagacagtggcatgcccagtgttgcacagtaagtgtgtggtaaagccgagattcaaactcagaccttctggccccttgcctaggagagcatgcccagttgtctagcagattctcttttgtctgagtggcccagatgacatctcttttagagctagaaagaaggagaaatgagacagggtctttgggctggagcctcctgggactaacatggcactggtcggtttgccaggcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacatangaaagcctgtgtgcatggcgggaagacgtactcccacggggaggtgtggcacccggccttccgtgccttcggcccttgcccatgcatcctatgcacctgtgaggatggccgccaggactgccagcgtgtgacctgtcccacgaagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 78 - DNA, 1722 bp, Homo sapiens
accttccccctttctttgatcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcgggctcacaacctgccccgaaccaggctgcccagcacccctcccgctgccagactcctgctgccaggcctgcaaagatgaggcaagtgagcaatcggatgaagaggaccgtgtgcagtcgctccatggggtgagacatcctcaggatccatgttccagtgatgctgggagaaagagaggcccgggcaccccagcccccactggcctcagcgcccctctgagcttcatccctcgccacttcatacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacctggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgctgttattatatattaataaataagaagttgcataaccatcaaaa

SEQ ID NO: 79 - DNA, 1722 bp, Homo sapiens
accttccccctttctttgatcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaggcctgcaaaggtgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcataaccatcaaaa

SEQ ID NO: 80 - DNA, 1515 bp, Homo sapiens; identical to SEQ ID NO: 10

SEQ ID NO: 81 - DNA, 1817 bp, Homo sapiens; identical to SEQ ID NO: 7

SEQ ID NO: 82 - DNA, 1622 bp, Homo sapiens; identical to SEQ ID NO: 8

SEQ ID NO: 83 - DNA, 1567 bp, Homo sapiens; identical to SEQ ID NO: 9

SEQ ID NO: 84 - DNA, 1202 bp, Mouse
  misc_feature (57)..(57): n is any nucleic acid
atttctctattcctgatcccacactgctctgcctacccacaccagccccaaggtctnagaaagccctggaggctggcttgccaaatccttgtcagtgtntttattgattagtctgagaatatcttagacctcacccacaaggttctgtgtggagcctgtgctctctgtctgtctgtctgtctgtctgtctgtctgtctgtctgcctgcctctctctgtctgtctccgtctgtctctgtctctctgtctgtctctgtctgtctctttctctctgtctctctctgtgtctctgtctctgtctctgtctctctctctctctcagaagtcctctagccttctctagcaggcgtctcatgcagcctggttggtgttcccagctgtggcctatcccacagacagctccacatcctgcttgctgttcgcagagacattcccaggatccatgctcggagaggagaggccccagcacgccagcccccaccagcctcagctcccctctgggcttcatccntcgccacttccagtcagtaggaatgggcagcacaaccatcaagattatcttgaaggagaaacataaaaaagcttgcacacacaatgggaagacatactcccatggggaggtgtggcaccccactgtgctctcctttggccccatgccctgcatcctgtgcacatgtattgatggctaccaggactgccaccgtgtgacctgccccacccaatatccctgcagtcaacccaagaaagtggctgggaagtgctgcaagatctgcccagaggacgaggcggaagatgaccacagtgaggtcatttccacccggtgtcccaaggtaccaggccagttccaggtgtacacgttggcatctccaagcccagacagcctacaccgctttgtcctggagcatgaagcctctgaccaggtagagatgtacatttggaagctggtgaaaggaatttaccacttggttcagatcaagagagtcaggaagcaagatttccagaaagaggttcagaacttccggctgctcaccggcacccatgaaggttactggaccgttttcctagcccagattccagagctgaaagttacagccagcccagacaaagtgaccaagacattatagcaaggacctaaagagttgcagatacgagttttattggttttgttattatatattaataaagaagtcgcattaccctttc

SEQ ID NO: 85 - protein, 398 aa, Homo sapiens
  misc_feature (200)..(200): Xaa is any amino acid
REPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHXKACVHGGKTYSHGEVWHPAFRAFGPCPCILCTCEDGRQDCQRVTCPTKYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNFHLTQIKKVRKQDFQKEAQHFRLLAGPHEGHWNVFLAQTLELKVTASPDKVTKT

SEQ ID NO: 86 - protein, 539 aa, Homo sapiens
  misc_feature (69)..(69): Xaa is any amino acid
SPLPSAGPSFVSPSLPPFPAFSFHLSLLPTLDLPSCPPFLPTAASWPFSDPALAADLLGSCGLICGPCXSVSFSSPVLPTPLPDQRPDPGERMVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHXKACVHGGKTYSHGEVWHPAFRAFGPCPCILCTCEDGRQDCQRVTCPTKYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNFHLTQIKKVRKQDFQKEAQHFRLLAGPHEGHWNVFLAQTLELKVTASPDKVTKT

SEQ ID NO: 87 - protein, 388 aa, Homo sapiens
  misc_feature (190)..(190): Xaa is any amino acid
ISSWGQMQNHQKSGLVNFSKDSHETSFSSSSCPSPTVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHXKACVHGGKTYSHGEVWHPAFRAFGPCPCILCTCEDGRQDCQRVTCPTKYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNFHLTQIKKVRKQDFQKEAQHFRLLAGPHEGHWNVFLAQTLELKVTASPDKVTKT

SEQ ID NO: 88 - protein, 439 aa, Homo sapiens
  misc_feature (241)..(241): Xaa is any amino acid
DRVFGLEPPGTNMALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHXKACVHGGKTYSHGEVWHPAFRAFGPCPCILCTCEDGRQDCQRVTCPTKYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNFHLTQIKKVRKQDFQKEAQHFRLLAGPHEGHWNVFLAQTLELKVTASPDKVTKT

SEQ ID NO: 89 - protein, 549 aa, Homo sapiens [sequence omitted]

SEQ ID NO: 90 - protein, 549 aa, Homo sapiens
TFPLSLIASPFCWTFLRLSISPSFPRVLFPPFSSSHLRPPFLPSFPAHRCFLALLRPRSSSRPPGVCGLICGPCASVSFSSPFLPTPLPDQRPDPGERMVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKGEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 91 - protein, 392 aa, Homo sapiens
ISSWGQMQNHQKSGLVNFSKDSHETSFSSSSCPSPTAEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 92 - protein, 443 aa, Homo sapiens
DRVFGLEPPGTNMALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 93 - protein, 378 aa, Homo sapiens
DRVFGLEPPGTNMALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT

SEQ ID NO: 94 - protein, 356 aa, Homo sapiens
DRVFGLEPPGTNMALVGLPGPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKEDKADPGHSEISSTRCPKAPGRVLVHTSVSPSPDNLRRFALEHEASDLVEIYLWKLVKGIFHLTQIKKVRKQDFQKEAQHFRLLAGPHEGHWNVFLAQTLELKVTASPDKVTKT

SEQ ID NO: 95 - protein, 397 aa, Mouse
  misc_feature (18)..(18): Xaa is any amino acid
FLYSSHTALPTHTSPKVXESPGGWLAKSLSVXLLISLRISTSPTRFCVEPVLSVCLSVCLSVCLSACLSLSVSVCLCLSVCLCLSLSLCLSLCLCLCLCLSLSLRSPLAFSSRRLMQPGWCSQLWPIPQTAPHPACCSQRHSQDPCSERRGPSTPAPTSLSSPLGFIXRHFQSVGMGSTTIKIILKEKHKKACTHNGKTYSHGEVWHPTVLSFGPMPCILCTCIDGYQDCHRVTCPTQYPCSQPKKVAGKCCKICPEDEAEDDHSEVISTRCPKVPGQFQVYTLASPSPDSLHRFVLEHEASDQVEMYIWKLVKGIYHLVQIKRVRKQDFQKEVQNFRLLTGTHEGYWTVFLAQIPELKVTASPDKVTKTLQGPKELQIRVLLVLLLYINKEVALPF